(19) United States
(12) Patent Application Publication
(10) Pub. No.: US 2017/0348405 A1
(43) Pub. Date: Dec. 7, 2017

(54) MODIFICATION OF RECOMBINANT ADENOVIRUS WITH IMMUNOGENIC PLASMODIUM CIRCUMSPOROZOITE PROTEIN EPITOPES

(71) Applicant: THE ROCKEFELLER UNIVERSITY, New York, NY (US)

(72) Inventors: Takayuki Shiratsuchi, Tokushima (JP); Moriya Tsuji, New York, NY (US)

(21) Appl. No.: 15/419,523

(22) Filed: Jan. 30, 2017

Related U.S. Application Data
(63) Continuation of application No. 13/399,654, filed on Feb. 17, 2012, now Pat. No. 9,555,089.
Application No. 13/399,654 is a continuation of application No. PCT/US10/45952, filed on Aug. 18, 2010.
Application No. PCT/US10/45952 is a continuation-in-part of application No. PCT/US09/54212, filed on Aug. 18, 2009.

(51) Int. Cl. A61K 39/015; C07K 14/005; C12N 15/861; A61K 39/00

(52) U.S. Cl. CPC A61K 39/015; C12N 15/861; C07K 14/005; C12N 2710/10322; A61K 2039/5256; C07K 2319/00; A61K 2039/55577; C12N 2710/10341; C12N 2710/10343; C07K 2319/40

(57) ABSTRACT

The present disclosure relates to adenovirus protein modifications to augment immune response to a transgene of a recombinant adenovirus and to circumvent pre-existing anti-adenovirus immunity. Some embodiments are directed to a recombinant adenovirus derived from a recombinant adenovirus plasmid vector, wherein the recombinant adenovirus plasmid vector comprises a nucleotide sequence encoding a Plasmodium circumsporozoite protein, or antigenic portion thereof, operably linked to a heterologous promoter and a modified capsid or core protein, wherein an immunogenic epitope of Plasmodium circumsporozoite is inserted into or replaces at least part of a capsid or core protein. Other embodiments are directed to a pharmaceutical composition or a malaria vaccine composition comprising a recombinant adenovirus according to the above embodiments. Further embodiments include a method of treating, preventing, or diagnosing malaria, comprising administering a therapeutic amount of the pharmaceutical composition or malaria vaccine composition in accordance with the above embodiment.

### CROSS-REFERENCE TO RELATED APPLICATIONS

This application is a continuation of U.S. application Ser. No. 13/399,654, filed Feb. 17, 2012 and now pending, which is a continuation of International Application No. PCT/US10/045952, filed Aug. 18, 2010, which claims priority to and is a continuation-in-part of International Application No. PCT/US09/054212, filed on Aug. 18, 2009, all of which are incorporated by reference in their entirety, as if fully set forth herein.

### STATEMENT REGARDING FEDERALLY SPONSORED RESEARCH

This invention was made with government support under Grant No. 1R01A1081510-01A1 awarded by the National Institute of Allergy and Infectious Diseases (NIAID), an institute that is part of the National Institutes of Health. The government has certain rights in the invention.

### FIELD OF THE INVENTION

The invention relates to the field of medicine and biotechnology. More particularly, the invention relates to the use of capsid-modified adenoviral vectors to induce a potent immune response to a malaria parasite antigen such as Plasmodium circumsporozoite protein, which are suitable for vaccines against malaria.

### BACKGROUND

Malaria is a severe disease that ranks among the most prevalent infections in tropical areas throughout the world. Approximately 300-500 million people become infected yearly, with relatively high rates of morbidity and mortality. Severe morbidity and mortality occur particularly in young children and in adults migrating to a malaria endemic area without having undergone prior malaria exposure. The World Health Organization (WHO) estimates that 2-3 million children die of malaria in Africa alone, every year. The widespread occurrence and the increasing incidence of malaria in many countries, caused by drug-resistant parasites (Plasmodium falciparum, recently also Plasmodium vivax) and insecticide-resistant vectors (Anopheles mosquitoes), underscore the need for developing new methods for the control of this disease (Nussenzweig and Long 1994).

Malaria parasites have a complicated life cycle consisting of pre-erythrocytic, erythrocytic and sexual parasitic forms, representing a potential target for the development of a malaria vaccine. The pre-erythrocytic and erythrocytic forms are found in the host, while the sexual forms occur in the vector. Immunization with live-attenuated irradiated sporozoites (IrSp) has been shown to induce sterile protection (i.e., complete resistance against parasite challenge) in mice (Nussenzweig et al. 1967), non-human primates (Gwadz et al. 1979) and human (Clyde et al. 1973, Edelman et al. 1993). Protection conferred by IrSp is mediated by sporozoite neutralization by both humoral (B cell) and cellular (T cell) immune responses (Tsuji et al. 2001). Although an IrSp vaccination is an attractive solution, the only way to obtain sporozoites is by dissecting mosquito salivary glands, and there is currently no known technology to grow large numbers of sporozoites in vitro. Therefore, an alternate vaccine vector that can elicit an equally strong protective immunity against malaria is needed.

One promising target for such a vaccine vector is the circumsporozoite (CS) protein, which is expressed on the surface of the sporozoite. Effective neutralizing antibodies are directed against the immunodominant, species specific, repeat domains of the circumsporozoite (CS) protein. In Plasmodium falciparum (human malaria parasite), the repeats (NANP)ₙ are conserved among isolates from all areas of the world. This central repeat contains multiple repeat of B cell epitopes, and, therefore, the CS protein can induce a strong humoral immune response by triggering B cells (Tsuji et al. 2001). At the C-terminal region of the CS protein, there are several T cell epitopes, which can induce a significant cellular immune response (Tsuji et al. 2001). The humoral (antibody) response can eliminate parasites by interacting and neutralizing the infectivity of sporozoites (extra-cellular parasite) prior to entering hepatocyte, whereas the cellular (T cell) response can attack EEF (an intra-cellular parasite) by secreting interferon-gamma. These immune responses prevent the EEFs from maturing and dividing rapidly to form thousands of merozoites that reenter the blood and infect erythrocytes causing the disease we recognize as malaria.

One CS-based malarial vaccine that is currently undergoing human trials is GlaxoSmithKline's RTS, S, fusion protein of the Hepatitis B surface antigen and a portion of Plasmodium falciparum circumsporozoite protein (PfCSP) in a form of virus-like particle (International Patent Application No. PCT/EP1992/002591 to SmithKline Beecham Biologicals S.A., filed Nov. 11, 1992), has been shown to decrease malaria infection in clinical trials (Alonso et al. 2004, Alonso et al. 2005, Bejon et al. 2008). RTS, S induces an anti-PfCSP humoral immune response, but a relatively weak PfCSP-specific cellular (CD8+) response (Kester et al. 2008), which might be the reason for the relatively weak protection by RTS, S. In contrast, adenovirus-based malaria vaccines can induce a protective cellular immune response (International Patent Application No. PCT/EP2003/051019, filed Dec. 16, 2003, Rodrigues et al. 1997). However, there are currently two obstacles that limit the use of an adenovirus-based platform as a malaria vaccine: (1) lack of a capability of inducing a potent humoral response against a transgene product, and (2) pre-existing immunity to adenovirus, especially adenovirus serotype 5, which hampers the immunogenicity of adenovirus-based vaccine.

One approach that has recently been taken in an attempt to augment adenovirus-induced humoral response is to insert a B cell antigenic epitope (e.g., a bacterial or viral epitope) in adenovirus capsid proteins such as Hexon, Fiber, Penton and pIX (Worgall et al. 2005, McConnell et al. 2006, Krause et al. 2006, Worgall et al. 2007).

In addition, to circumvent pre-existing immunity to adenovirus serotype 5 (Ad5), other adenovirus serotypes with lower seroprevalence, such as adenovirus serotype 11, 35, 26, 48, 49 and 50, have been evaluated as a vaccine platform and shown to induce immune response to a transgene in spite of the presence of anti-Ad5 immunity (International Patent Application No. PCT/EP2005/055183 to Crucell Holland B.V., filed Oct. 12, 2005, Abbink et al. 2007). Substitution of Ad5 Hexon, which is the target capsid protein of neutralizing antibody, with that of other serotypes has also been constructed in order to escape pre-existing anti-Ad5 immunity (Wu et al. 2002, Roberts et al. 2006).

There is, however, no improved adenoviral vector reported to have overcome the two obstacles at the same time in applying an adenoviral vector to a malaria vaccine mentioned above. Given that seroprevalence to Ad5 is high in malaria endemic areas (Ophorst et al. 2006.), there is a need for an adenovirus-based malaria vaccine that induces both protective humoral and cellular immune responses even in the presence of pre-exiting immunity to adenovirus.

### SUMMARY

The present disclosure relates to various adenovirus protein modifications to augment immune response to a transgene of a recombinant adenoviral vaccine and to circumvent pre-existing anti-adenovirus immunity.

More specifically, one embodiment is directed to a recombinant adenovirus derived from a recombinant adenovirus plasmid vector, wherein the recombinant adenovirus plasmid vector comprises a nucleotide sequence encoding (i) a Plasmodium circumsporozoite protein, or antigenic portion thereof, operably linked to a heterologous promoter: and (ii) a modified capsid or core protein, wherein an immunogenic epitope of Plasmodium circumsporozoite has been inserted into or replaces at least part of a capsid or core protein.

In some embodiments, the Plasmodium circumsporozoite protein further comprises a Plasmodium falciparum or Plasmodium yoelii circumsporozoite protein. The circumsporozoite protein may further comprise a codon-optimized Plasmodium falciparum or Plasmodium yoelii circumsporozoite protein, and in some aspects, may be encoded by the nucleotide sequence of SEQ ID NO:2 or SEQ ID NO:1, respectively.

In other embodiments, the immunogenic epitope further comprises a B cell epitope of Plasmodium circumsporozoite protein. The B cell epitope may be incorporated in a modified capsid protein, and in some aspects, the capsid protein may comprise a Hexon hypervariable region (HVR). The HVR may further comprise HVR1 or HVR5, wherein a portion of HVR1 or HVR5 is replaced with the B cell epitope. In other aspects, the capsid protein may further comprise a capsid Fiber protein wherein the B cell epitope is inserted into the Fiber protein. In some aspects, the B cell epitope is a Plasmodium falciparum circumsporozoite protein B cell epitope, wherein the B cell epitope is a repeat sequence, for example, (NANP)ₙ (SEQ ID NO:60), wherein the repeat sequence may be (NANP)₄, (NANP)₆, (NANP)₈, (NANP)₁₀, (NANP)₁₂, (NANP)₁₄, (NANP)₁₆, (NANP)₁₈, (NANP)₂₀, (NANP)₂₂ or (NANP)₂₈. In other aspects, the B cell epitope is a Plasmodium yoelii circumsporozoite protein B cell epitope, wherein the B cell epitope is a repeat sequence, for example, (QGPGAP)ₙ (SEQ ID NO:59), wherein the repeat sequence may be (QGPGAP)₃, (QGPGAP)₄, (QGPGAP)₅, (QGPGAP)₆, (QGPGAP)₇, (QGPGAP)₈, (QGPGAP)₉, (QGPGAP)₁₁, or (QGPGAP)₁₂.

In yet other embodiments, the immunogenic epitope further comprises a CD4+ or CD8+ T cell epitope of Plasmodium circumsporozoite protein. The CD4+ or CD8+ T cell epitope may be incorporated in a modified capsid or core protein. In some aspects, the capsid protein may comprise a Hexon hypervariable region (HVR). The HVR may further comprise HVR1 wherein a portion of HVR1 is replaced with the CD4+ or CD8+ T cell epitope. In other aspects, the core protein further comprises a pVII protein and a CD4+ T cell epitope is inserted into the pVII protein. In some aspects the CD4+ T cell epitope is a Plasmodium falciparum circumsporozoite CD4+ T cell epitope, wherein the CD4+ T cell epitope is EYLNKIQNSLSTEWSPCSVT (SEQ ID NO:62). In other aspects the CD4+ T cell epitope is a Plasmodium yoelii circumsporozoite CD4+ T cell epitope, wherein the CD4+ T cell epitope is YNRNIVNRLLGDALNGKPEEK (SEQ ID NO:61)

Other embodiments are directed to a pharmaceutical composition or malaria vaccine composition comprising a recombinant adenovirus according to the above embodiments. Further embodiments include a method of treating, preventing, or diagnosing malaria, comprising administering a therapeutic amount of the pharmaceutical composition or malaria vaccine composition in accordance with the above embodiments.

In another embodiment, a method for treatment comprising administering a prime-boost vaccination, wherein a subject is given a series of increasing dosages or same dosages at a given time interval. The time interval may be any length sufficient to generate a humoral and/or cellular immune response. For example, as described below, the interval may be, but is not limited to, once every 3 weeks.

### BRIEF DESCRIPTION OF THE DRAWINGS

FIG. 1 is a schematic diagram of a capsid-modified recombinant adenovirus in accordance with embodiments of the disclosure.

FIG. 2 is a schematic diagram illustrating the construction of the HVR1-Modified Adenovirus DNA of an HVR1-modified recombinant adenovirus plasmid vector.

FIG. 3 is a schematic diagram illustrating the construction of the HVR5-Modified Adenovirus DNA of an HVR5-modified recombinant adenovirus plasmid vector.

FIG. 4 is a schematic diagram illustrating the construction of the Fiber-Modified Adenovirus DNA of a Fiber-modified recombinant adenovirus plasmid vector.

FIG. 5 is a schematic diagram illustrating the construction of the HVR1 and Fiber-Modified Adenovirus DNA of an HVR1 and Fiber-modified recombinant adenovirus plasmid vector.

FIG. 6 is a schematic diagram illustrating the construction of the Fiber and pVII-Modified Adenovirus DNA of a Fiber and pVII-modified recombinant adenovirus plasmid vector.

FIG. 7 is a schematic diagram illustrating the construction of the HVR1 and pVII-Modified Adenovirus DNA of an HVR1 and pVII-modified recombinant adenovirus plasmid vector.

FIG. 8 is a schematic diagram illustrating the construction of the HVR1, Fiber and pVII-Modified Adenovirus DNA of an HVR1, Fiber and pVII-modified recombinant adenovirus plasmid vector.

FIG. 9 is the nucleic acid sequence of codon-optimized Plasmodium yoelii circumsporozoite protein (PyCS, SEQ ID NO:1) and the corresponding amino acid sequence (SEQ ID NO:30)

FIG. 10 is the nucleic acid sequence of codon-optimized Plasmodium falciparum circumsporozoite protein (PfCSP, SEQ ID NO:2) and the corresponding amino acid sequence (SEQ ID NO:43)

FIGS. 11A-C is the nucleic acid and amino acid sequences of a modified Hexon having three repeats of the PyCS B cell epitope sequence (QGPGAP)ₙ, (SEQ ID NO:59; n=3) in HVR1 (SEQ ID NO:3, nucleic acid; SEQ ID NO:31, amino acid). The inserted (QGPGAP)₃ sequence is underlined.

FIGS. 12A-C is the nucleic acid and amino acid sequences of a modified Hexon having four repeats of the PyCS B cell epitope sequence (QGPGAP)ₙ, (SEQ ID NO:59; n=4) in HVR1 (SEQ ID NO:4, nucleic acid; SEQ ID NO: 32, amino acid). The inserted (QGPGAP)₄ sequence is underlined.

FIGS. 13A-C is the nucleic acid and amino acid sequences of modified Hexon having five repeats of the PyCS B cell epitope sequence (QGPGAP)ₙ, (SEQ ID NO:59; n=5) in HVR1 (SEQ ID NO:5, nucleic acid; SEQ ID NO: 33, amino acid). The inserted (QGPGAP)₅ sequence is underlined.

FIGS. 14A-C is the nucleic acid and amino acid sequences of modified Hexon having six repeats of the PyCS B cell epitope sequence (QGPGAP)ₙ, (SEQ ID NO:59; n=6) in HVR1 (SEQ ID NO:6, nucleic acid; SEQ ID NO: 34, amino acid). The inserted (QGPGAP)₆ sequence is underlined.

FIGS. 15A-C is the nucleic acid and amino acid sequences of modified Hexon having seven repeats of the PyCS B cell epitope sequence (QGPGAP)ₙ, (SEQ ID NO:59; n=7) in HVR1 (SEQ ID NO:7, nucleic acid; SEQ ID NO: 35, amino acid). The inserted (QGPGAP)₇ sequence is underlined.

FIGS. 16A-C is the nucleic acid and amino acid sequences of modified Hexon having eight repeats of the PyCS B cell epitope sequence (QGPGAP)ₙ, (SEQ ID NO:59; n=8) in HVR1 (SEQ ID NO:8, nucleic acid; SEQ ID NO: 36, amino acid). The inserted (QGPGAP)₈ sequence is underlined.

FIGS. 17A-C is the nucleic acid and amino acid sequences of modified Hexon having nine repeats of the PyCS B cell epitope sequence (QGPGAP)ₙ, (SEQ ID NO:59; n=9) in HVR1 (SEQ ID NO:9, nucleic acid; SEQ ID NO: 37, amino acid). The inserted (QGPGAP)₉ sequence is underlined.

FIGS. 18A-C is the nucleic acid and amino acid sequences of modified Hexon having eleven repeats of the PyCS B cell epitope sequence (QGPGAP)ₙ, (SEQ ID NO:59; n=11) in HVR1 (SEQ ID NO:10, nucleic acid; SEQ ID NO: 38, amino acid). The inserted (QGPGAP)ᵢᵢ sequence is underlined.

FIGS. 19/A-C is the nucleic acid and amino acid sequences of modified Hexon having twelve repeats of the PyCS B cell epitope sequence (QGPGAP)ₙ, (SEQ ID NO:59; n=12) in HVR1 (SEQ ID NO:11, nucleic acid; SEQ ID NO: 39, amino acid). The inserted (QGPGAP)₁₂ sequence is underlined.

FIGS. 20A-C is the nucleic acid and amino acid sequences of modified Hexon having four repeats of the PfCSP B cell epitope sequence (NANP)ₙ, (SEQ ID NO:60; n=4) in HVR1 (SEQ ID NO:12, nucleic acid; SEQ ID NO: 44, amino acid). The inserted (NANP)₄ sequence is underlined.

FIGS. 21A-C is the nucleic acid and amino acid sequences of modified Hexon having six repeats of the PfCSP B cell epitope sequence (NANP)ₙ, (SEQ ID NO:60; n=6) in HVR1 (SEQ ID NO:13, nucleic acid; SEQ ID NO: 45, amino acid). The inserted (NANP)₆ sequence is underlined.

FIGS. 22A-C is the nucleic acid and amino acid sequences of modified Hexon having eight repeats of the PfCSP B cell epitope sequence (NANP)ₙ, (SEQ ID NO:60; n=8) in HVR1 (SEQ ID NO:14, nucleic acid; SEQ ID NO: 46, amino acid). The inserted (NANP)₈ sequence is underlined.

FIGS. 23A-C is the nucleic acid and amino acid sequences of modified Hexon having ten repeats of the PfCSP B cell epitope sequence (NANP)ₙ, (SEQ ID NO:60; n=10) in HVR1 (SEQ ID NO:15, nucleic acid; SEQ ID NO: 47, amino acid). The inserted (NANP)₁₀ sequence is underlined.

FIGS. 24A-C is the nucleic acid and amino acid sequences of modified Hexon having twelve repeats of the PfCSP B cell epitope sequence (NANP)ₙ, (SEQ ID NO:60; n=12) in HVR1 (SEQ ID NO:16, nucleic acid; SEQ ID NO: 48, amino acid). The inserted (NANP)₁₂ sequence is underlined.

FIGS. 25A-C is the nucleic acid and amino acid sequences of modified Hexon having fourteen repeats of the PfCSP B cell epitope sequence (NANP)ₙ, (SEQ ID NO:60; n=14) in HVR1 (SEQ ID NO:17, nucleic acid; SEQ ID NO: 49, amino acid). The inserted (NANP)₁₄ sequence is underlined.

FIGS. 26A-C is the nucleic acid and amino acid sequences of modified Hexon having sixteen repeats of the PfCSP B cell epitope sequence (NANP)ₙ, (SEQ ID NO:60; n=16) in HVR1 (SEQ ID NO:18, nucleic acid; SEQ ID NO: 50, amino acid). The inserted (NANP)₁₆ sequence is underlined.

FIGS. 27A-C is the nucleic acid and amino acid sequences of modified Hexon having eighteen repeats of the PfCSP B cell epitope sequence (NANP)ₙ, (SEQ ID NO:60; n=18) in HVR1 (SEQ ID NO:19, nucleic acid; SEQ ID NO: 51, amino acid). The inserted (NANP)₁₈ sequence is underlined.

FIGS. 28A-C is the nucleic acid and amino acid sequences of modified Hexon having twenty repeats of the PfCSP B cell epitope sequence (NANP)ₙ, (SEQ ID NO:60; n=20) in HVR1 (SEQ ID NO:20, nucleic acid; SEQ ID NO: 52, amino acid). The inserted (NANP)₂₀ sequence is underlined.

FIGS. 29A-C is the nucleic acid and amino acid sequences of modified Hexon having twenty-two repeats of the PfCSP B cell epitope sequence (NANP)ₙ, (SEQ ID NO:60; n=22) in HVR1 (SEQ ID NO:21, nucleic acid; SEQ ID NO: 53, amino acid). The inserted (NANP)₂₂ sequence is underlined.

FIGS. 30A-C is the nucleic acid and amino acid sequences of modified Hexon having twenty-eight repeats of the PfCSP B cell epitope sequence (NANP)ₙ, (SEQ ID NO:60; n=28) in HVR1 (SEQ ID NO:22, nucleic acid; SEQ ID NO: 54, amino acid). The inserted (NANP)₂₈ sequence is underlined.

FIGS. 31A-C is the nucleic acid and amino acid sequences of modified Hexon having three repeats of the PyCS B cell epitope sequence (QGPGAP)ₙ, (SEQ ID NO:59; n=3) in HVR5 (SEQ ID NO:23, nucleic acid; SEQ ID NO:40, amino acid). The inserted (QGPGAP)₃ sequence is underlined.

FIGS. 32A-B is the nucleic acid and amino acid sequences of modified Fiber having three repeats of the PyCS B cell epitope sequence (QGPGAP)ₙ, (SEQ ID NO:59; n=3) in Fiber (SEQ ID NO:24, nucleic acid; SEQ ID NO:41, amino acid). The inserted (QGPGAP)₃ sequence is underlined.

FIGS. 33A-B is the nucleic acid and amino acid sequences of modified Fiber having four repeats of the PfCSP B cell epitope sequence (NANP)ₙ, (SEQ ID NO:60; n=4) in Fiber (SEQ ID NO:25, nucleic acid; SEQ ID NO:55, amino acid). The inserted (NANP)₄ sequence is underlined.

FIG. 34 is the nucleic acid and amino acid sequences of the modified pVII having the PyCS CD4+ epitope sequence YNRNIVNRLLGDALNGKPEEK, (SEQ ID NO:61) at the N-terminus of pVII (SEQ ID NO:26, nucleic acid; SEQ ID NO:42, amino acid). The inserted YNRNIVNRLLGDALNGKPEEK sequence is underlined.

FIG. 35 is the nucleic acid and amino acid sequences of the modified pVII having the PfCSP CD4+ epitope sequence EYLNKIQNSLSTEWSPCSVT, (SEQ ID NO:62) at the C-terminus of pVII (pVII-1; SEQ ID NO:27, nucleic acid; SEQ ID NO:56, amino acid). The inserted EYLNKIQNSLSTEWSPCSVT sequence is underlined.

FIG. 36 is the nucleic acid and amino acid sequences of the modified pVII having the PfCSP CD4+ epitope sequence EYLNKIQNSLSTEWSPCSVT, (SEQ ID NO:62) before the first Nuclear Localization Signal (NLS) of pVII (pVII-2; SEQ ID NO:28, nucleic acid; SEQ ID NO:57, amino acid). The inserted EYLNKIQNSLSTEWSPCSVT sequence is underlined.

FIG. 37 is the nucleic acid and amino acid sequences of the modified pVII having the PfCSP CD4+ epitope sequence EYLNKIQNSLSTEWSPCSVT, (SEQ ID NO:62) between the two NLSs of pVII (pVII-3; SEQ ID NO:29, nucleic acid; SEQ ID NO:58, amino acid). The inserted EYLNKIQNSLSTEWSPCSVT sequence is underlined.

FIG. 38 shows PyCS protein expression in AD293 cells after transient transfection with PyCS-GFP/pShuttle-CMV. PyCS protein was detected by western blotting using mouse monoclonal anti-PyCS antibody (9D3).

FIG. 39 shows the results of silver staining and western blotting (A) and ELISA assay (B) of the purified capsid-modified recombinant PyCS-GFP adenoviruses to confirm the (QGPGAP)₃ epitope (SEQ ID NO:59; n=3) insertion into adenovirus capsid proteins. In the ELISA assay, ELISA plates were coated directly with purified adenoviruses and the inserted epitope in adenovirus particles was detected with anti-PyCS antibody.

FIG. 40 shows the results of silver staining and western blotting (A) and ELISA assay (B) of the purified capsid-modified recombinant PyCS adenoviruses to confirm the (QGPGAP)ₙ epitope (SEQ ID NO:59) insertion into adenovirus capsid proteins. In the ELISA assay, ELISA plates were coated directly with purified adenoviruses and the inserted epitope in adenovirus particles was detected with anti-PyCS antibody.

FIG. 41 illustrates a single immunization regimen with capsid-modified PyCS adenoviruses having (QGPGAP)ₙ repeats (SEQ ID NO:59, n=3, 4, 5, 6, 9, 12) (A) and PyCS-specific CD8+ response two weeks after immunization (B).

FIG. 42 illustrates a prime and boost immunization regimen with capsid-modified PyCS adenoviruses having (QGPGAP)ₙ repeats (SEQ ID NO:59, n=3) (A), PyCS-specific humoral responses at week 10 (B), and malaria parasite burden in liver 42 hours after sporozoite challenge (C).

FIG. 43 illustrates anti-sporozoite antibody titer determined by indirect immunofluorescene assay (IFA) (A) and in vitro sporozoite neutralizing activity (B) of pooled serum samples prepared from mice given the regimen in FIG. 42.

FIG. 44 illustrates a prime and boost immunization regimen with capsid-modified PyCS adenoviruses having (QGPGAP)ₙ repeats (SEQ ID NO:59, n=4, 6) in HVR1(A), PyCS-specific humoral responses at week 9 (B), malaria parasite burden in liver 42 hours after sporozoite challenge (C), and in vitro sporozoite neutralizing activity of pooled serum samples (D). Mice were immunized with or without adjuvant.

FIG. 45 illustrates a prime and boost immunization regimen with capsid-modified PyCS adenoviruses having (QGPGAP)ₙ repeats (SEQ ID NO:59, n=6, 9, 12) in HVR1(A), PyCS-specific humoral responses at week 9 (B), PyCS-specific CD8+ T cell responses at week 9 (C), and malaria parasite burden in liver 42 hours after sporozoite challenge (D). Mice were immunized with or without adjuvant.

FIG. 46 shows PfCSP protein expression in AD293 cells after transient transfection with PfCSP/pShuttle-CMV. PfCSP was detected by western blotting using mouse monoclonal anti-NANP antibody (2A10).

FIG. 47 illustrates the results of silver staining and western blotting (A), and ELISA assay (B) of the purified capsid-modified recombinant PfCSP adenoviruses to confirm the (NANP)₄ epitope (SEQ ID NO:60; n=4) insertion into adenovirus capsid proteins. The inserted (NANP)₄ epitope (SEQ ID NO:60; n=4) was detected with mouse monoclonal anti-NANP antibody (2A10). In the ELISA assay, ELISA plates were coated directly with purified adenoviruses.

FIG. 48 shows the results of silver staining and western blotting (A) and ELISA assay (B) of the purified capsid-modified recombinant PfCSP adenoviruses to confirm the (NANP)ₙ epitope (SEQ ID NO:60; n=4, 6, 8, 10, 12, 14, 16, 18, 20, 22) insertion into adenovirus capsid proteins. In the ELISA assay, ELISA plates were coated directly with purified adenoviruses and the inserted epitope in adenovirus particles was detected with anti-PfCSP antibody (2A10).

FIG. 49 illustrates the prime and boast immunization regimen with capsid-modified recombinant PfCSP adenoviruses having (NANP)₄ (SEQ ID NO:60; n=4) (A) and PfCSP-specific humoral responses at week 9 (B).

FIG. 50 illustrates the prime and boast immunization regimen with capsid-modified recombinant PfCSP adenoviruses having (NANP)ₙ (SEQ ID NO:60; n=4, 6, 8, 10) in HVR1 (A) and PfCSP-specific humoral responses at week 9 (B).

FIG. 51 illustrates the prime and boast immunization regimen with capsid-modified recombinant PfCSP adenoviruses having (NANP)ₙ (SEQ ID NO:60; n=10, 16, 22) in HVR1 (A) and PfCSP-specific humoral responses at week 9 (B). Mice were immunized with or without adjuvant.

FIG. 52 illustrates the result of sliver staining analysis of purified (QGPGAP)₃-modified Fiber and pVII-1 ((QGPGAP)₃-Fib/CD4-pVII-1/PyCS-GFP) adenovirus (A) and anti-QGPGAP antibody titer at week 10 in mice immunized with (QGPGAP)₃-Fib/PyCS-GFP or (QGPGAP)₃-Fib/CD4-pVII-1/PyCS-GFP as described in FIG. 49 (B). The results of two independent experiments were plotted in the figure after normalization with the median antibody titers in B-Fib/PyCS-GFP-immunized group.

FIG. 53 shows schematic diagrams of the structure of the adenovirus pVII proteins with the PfCSP CD4+ epitope sequence EYLNKIQNSLSTEWSPCSVT (SEQ ID NO:62) inserted at the before the first Nuclear Localization Signal (NLS) of pVII (PfCD4-pVII-2; SEQ ID NO:28, nucleic acid; SEQ ID NO:57, amino acid) and between the two NLSs of pVII (PfCD4-pVII-3; SEQ ID NO:29, nucleic acid; SEQ ID NO:58, amino acid) (A) and the results of silver staining to confirm the epitope insertion into pVII (B).

FIG. 54 illustrates the prime and boast immunization regimen with HVR1 and pVII-modified recombinant PfCSP adenoviruses (A), PfCSP-specific humoral responses at week 6 (B), and PfCSP-specific CD4+(EYLNKIQNSLSTEWSPCSVT; SEQ ID NO:62) response at week 9 (C).

FIG. 55 illustrates in vitro neutralization of recombinant adenovirus by human serum samples. AD293 cells were infected with recombinant adenoviruses in the presence of diluted human serum for overnight and GFP expression was measured as a marker of infection.

FIG. 56 illustrates the effect of anti-adenovirus immunity on the induction of PyCS-specific T cell response by capsid-modified PyCS-GFP adenoviruses in vivo. (A) is the brief description of the study design. (B) shows PyCS-specific CD8+ T cell response in mice immunized with wild-type (wt)/empty adenovirus twice followed by priming with capsid-modified PyCS-GFP adenoviruses.

FIG. 57 illustrates the effect of anti-adenovirus immunity on the induction of PyCS-specific humoral immune response by capsid-modified PyCS-GFP adenoviruses in vivo. (A) is the brief description of the study design. (B) shows PyCS-specific humoral immune response in mice immunized with wild-type (wt)/empty adenovirus twice followed by two doses of capsid-modified PyCS-GFP adenoviruses.

### MEANS FOR SOLVING THE PROBLEMS

The present inventors have found a novel recombinant adenovirus having a novel, capsid-modified structure that is derived from a recombinant adenovirus plasmid vector. The recombinant adenovirus is capable of infecting mammalian cells, causing the cells to express a Plasmodium circumsporozoite protein. The recombinant adenovirus also has one or more capsid proteins that have been modified by having a desired immunogenic antigen, such as B cell epitope, T cell epitope of Plasmodium circumsporozoite protein. The recombinant adenovirus is obtained by the method of transfecting cells with the linearized recombinant adenovirus plasmid vector. Using the obtained recombinant adenovirus, the present inventors carried out extensive research on pharmaceuticals containing as an active ingredient a recombinant adenovirus having malaria infection preventive and therapeutic effects. As a result, the inventors found that the obtained recombinant adenovirus has the desired pharmaceutical effects.

### DETAILED DESCRIPTION

The following description provides specific details for a thorough understanding of, and enabling description for, embodiments of the disclosure. However, one skilled in the art will understand that the disclosure may be practiced without these details. In other instances, well-known structures and functions have not been shown or described in detail to avoid unnecessarily obscuring the description of the embodiments of the disclosure.

The abbreviations used for the amino acids, peptides, base sequences, and nucleic acids in the present disclosure are based on the abbreviations specified in the IUPAC-IUB Communication on Biochemical Nomenclature, Eur. J. Biochem., 138: 9 (1984), “Guideline for Preparing Specifications Including Base Sequences and Amino Acid Sequences” (United States Patent and Trademark Office), and those commonly used in this technical field.

A “nucleotide sequence,” “polynucleotide” or “DNA molecule” as contemplated by the current disclosure, may include double strand DNA or single strand DNA (i.e., a sense chain and an antisense chain constituting the double strand DNA), and is not limited to a full length thereof. Nucleotide sequences encoding an immunogenic foreign gene, such as those disclosed herein below, encompass double strand DNA containing genomic DNA, single strand DNA (sense chain) containing cDNA, single strand DNA (antisense chain) having a sequence complementary to the sense chain, synthetic DNA, and fragments thereof, unless otherwise mentioned.

Nucleotide sequences, polynucleotides or DNA molecules as used herein are not limited to the functional region, and may include at least one of an expression suppression region, a coding region, a leader sequence, an exon, and an intron. Further, examples of nucleotide sequences or polynucleotides may include RNA or DNA. A polypeptide containing a specific amino acid sequence and a polynucleotide containing a specific DNA sequence may include fragments, homologs, derivatives, and mutants of the polynucleotide. Examples of mutants of a nucleotide sequence or polynucleotide (such as mutant DNA), include naturally occurring allelic mutants; artificial mutants; and mutants having deletion, substitution, addition, and/or insertion. It should be understood that such mutants encode polypeptides having substantially the same function as the polypeptide encoded by the original non-mutated polynucleotide.

The present disclosure relates to a recombinant adenovirus that can express an antigenic determinant of a Plasmodium parasite, and comprises one or more modified capsid and/or core proteins. The recombinant adenovirus is derived from a recombinant adenovirus plasmid vector, the generation of which is described in the Examples below. The use of adenovirus as a vector is discussed further below. The recombinant adenovirus plasmid vectors described herein may be used as a malaria vaccine or pharmaceutical composition, wherein both humoral and cellular immune responses against the Plasmodium parasite are induced.

The Plasmodium parasite may be selected from any of the known Plasmodium (P.) species, for example, P. falciparum, P. malariae, P. ovale, P. vivax, P. knowlesi, P. berghei, P. chabaudi and P. yoelii. In some embodiments, the antigenic determinant is derived from the rodent-specific Plasmodium yoelii or the human-specific Plasmodium falciparum

In one embodiment, a recombinant adenovirus capsid-modified plasmid vector (also described as a recombinant adenovirus plasmid vector herein) is a plasmid that encodes and produces a capsid and/or core-modified recombinant adenovirus (also described as a recombinant adenovirus herein) that has a structure comprising one or more modified capsid and/or core proteins. In accordance with the embodiments of the disclosure, the modification of the capsid and/or core proteins may be accomplished by insertion of at least one immunogenic epitope of a Plasmodium circumsporozoite protein. Alternatively, at least part of the capsid and/or core protein may be deleted and replaced by at least one immunogenic epitope of a Plasmodium circumsporozoite protein. In some embodiments, the immunogenic epitope is a B-cell and/or T-cell epitope of a Plasmodium circumsporozoite protein. The addition of a B cell or T cell epitope may serve to enhance the efficacy of an adenoviral vector used as a malaria vaccine by establishing or enhancing the humoral immune response to the CS protein. The modified capsid and core proteins and their significance with respect to their use in the recombinant adenovirus described herein are discussed further below.

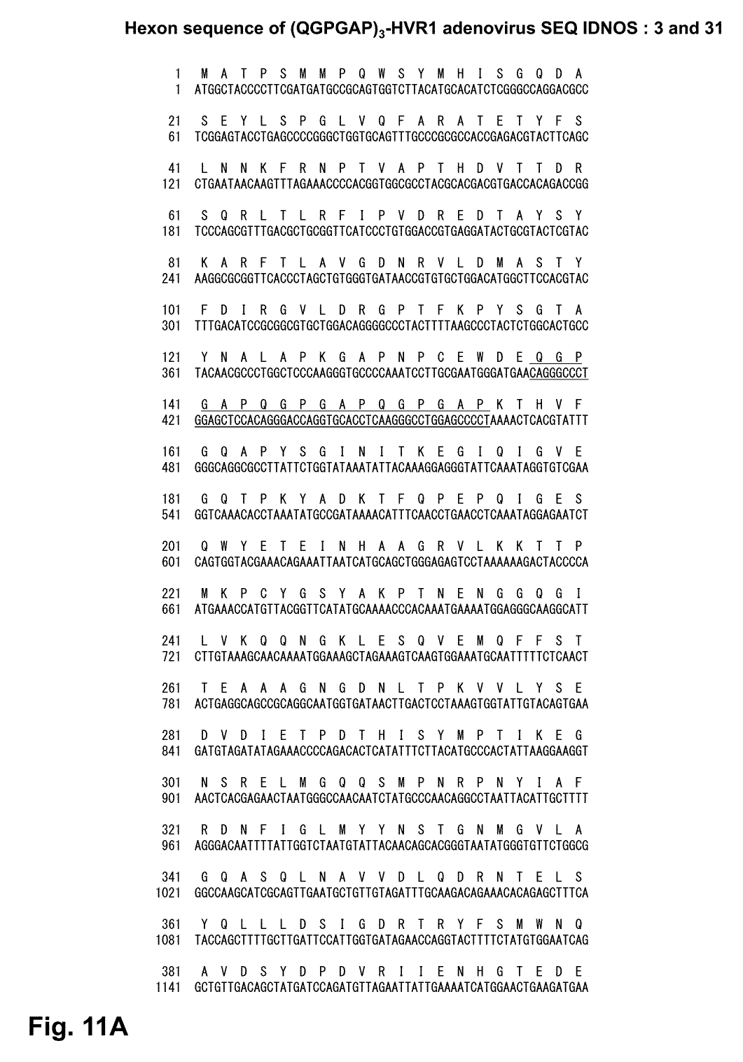

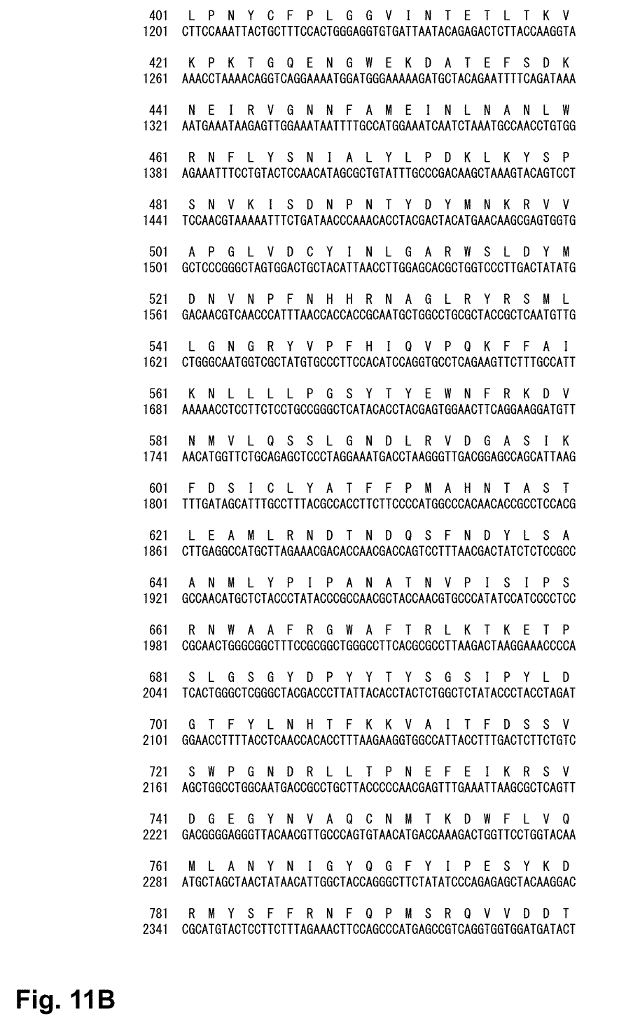

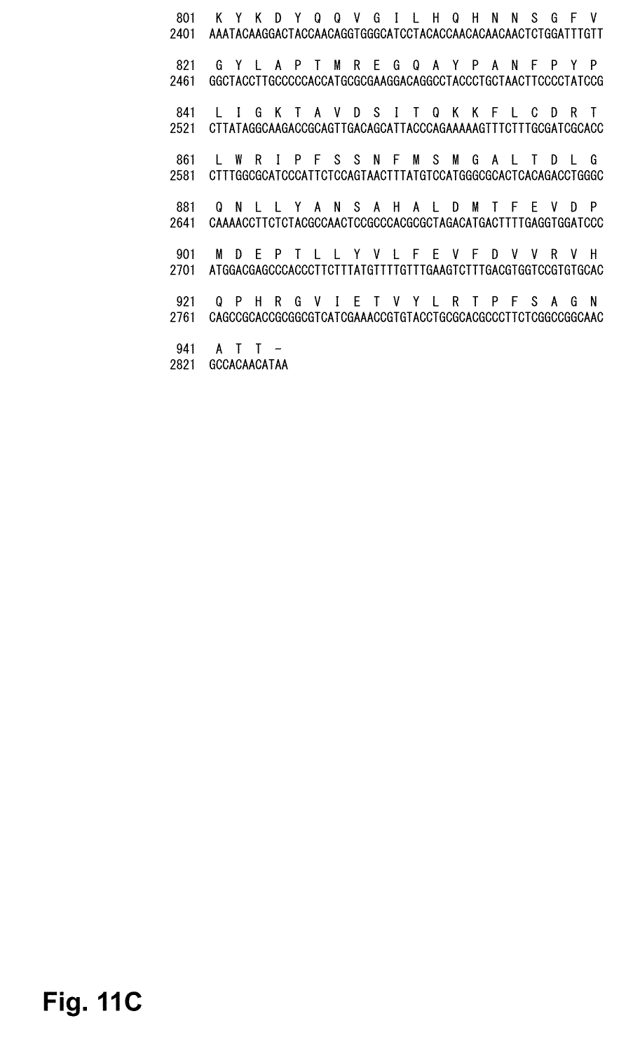

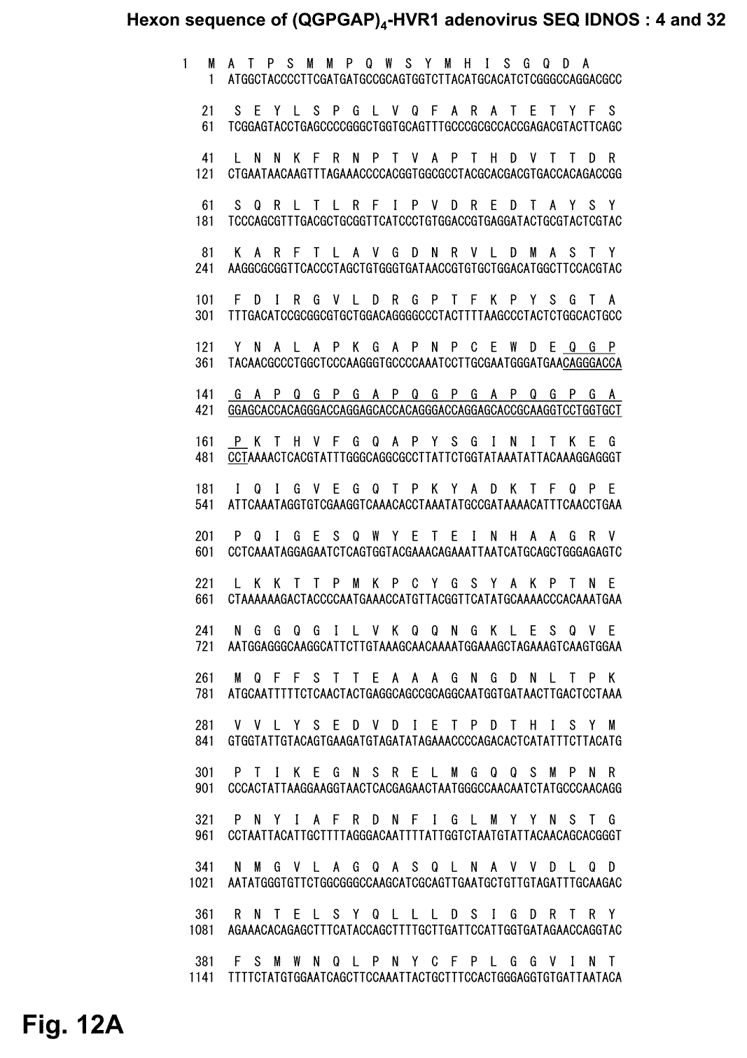

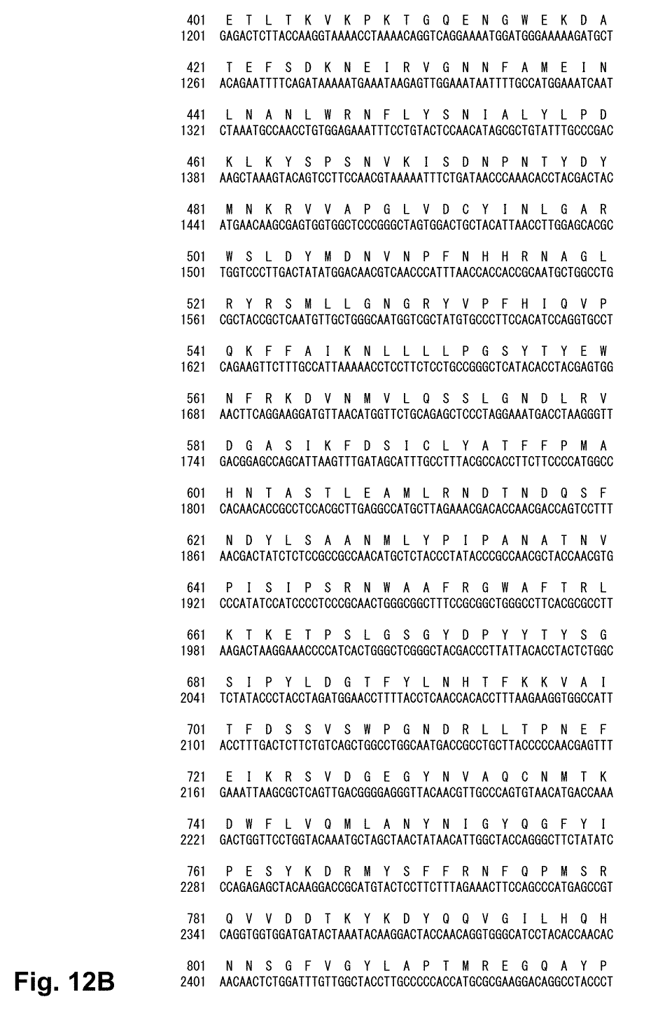

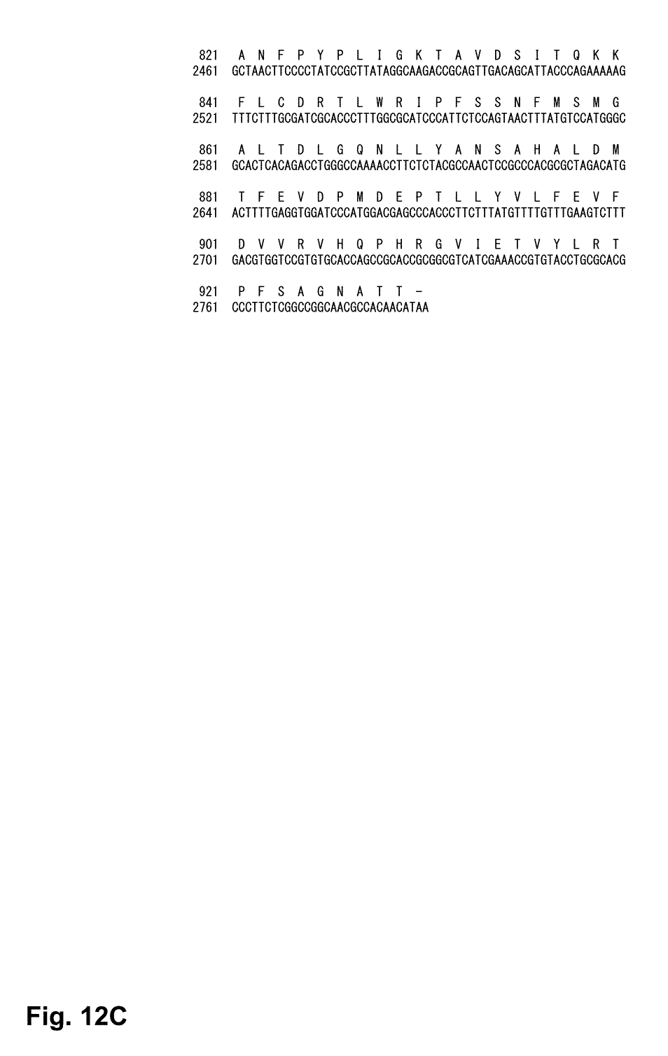

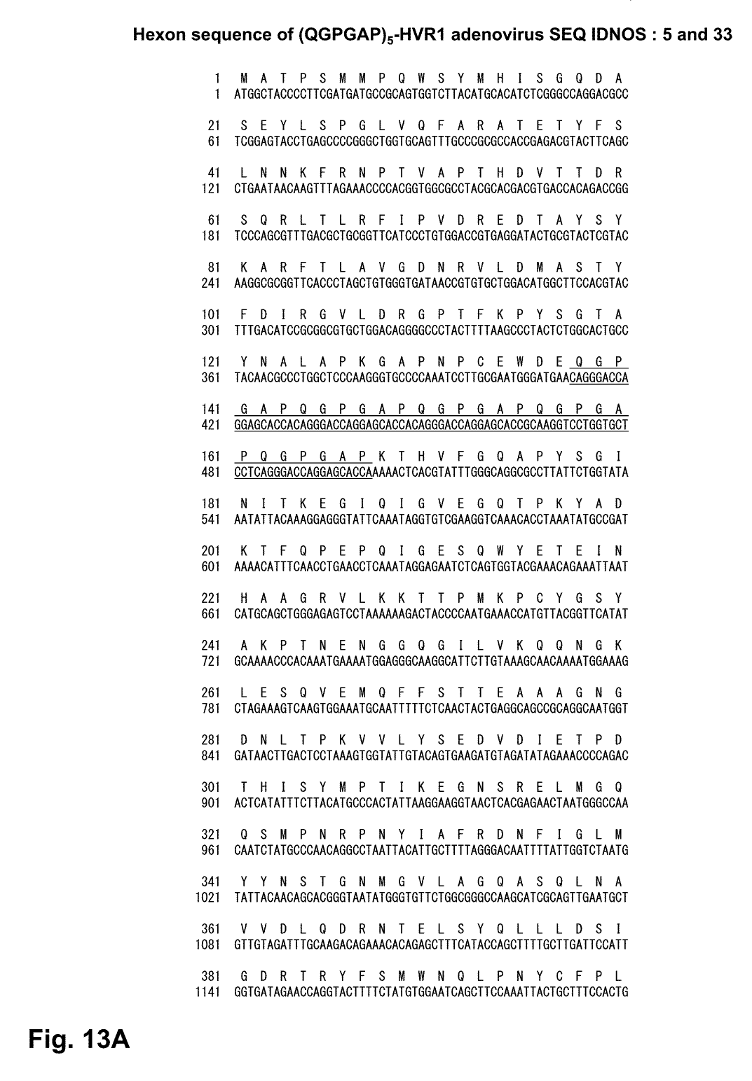

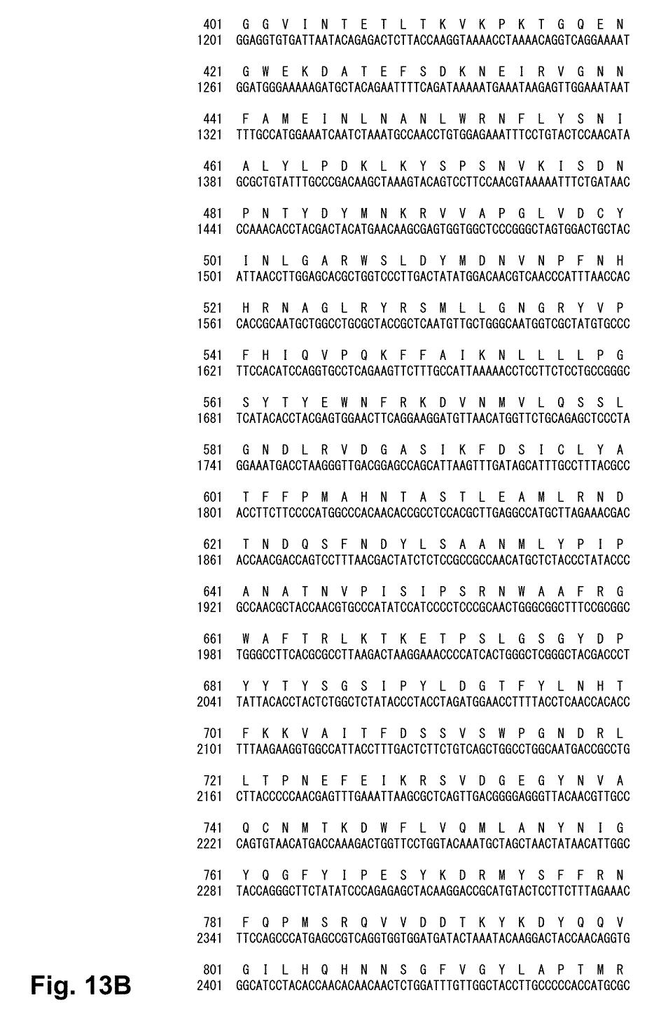

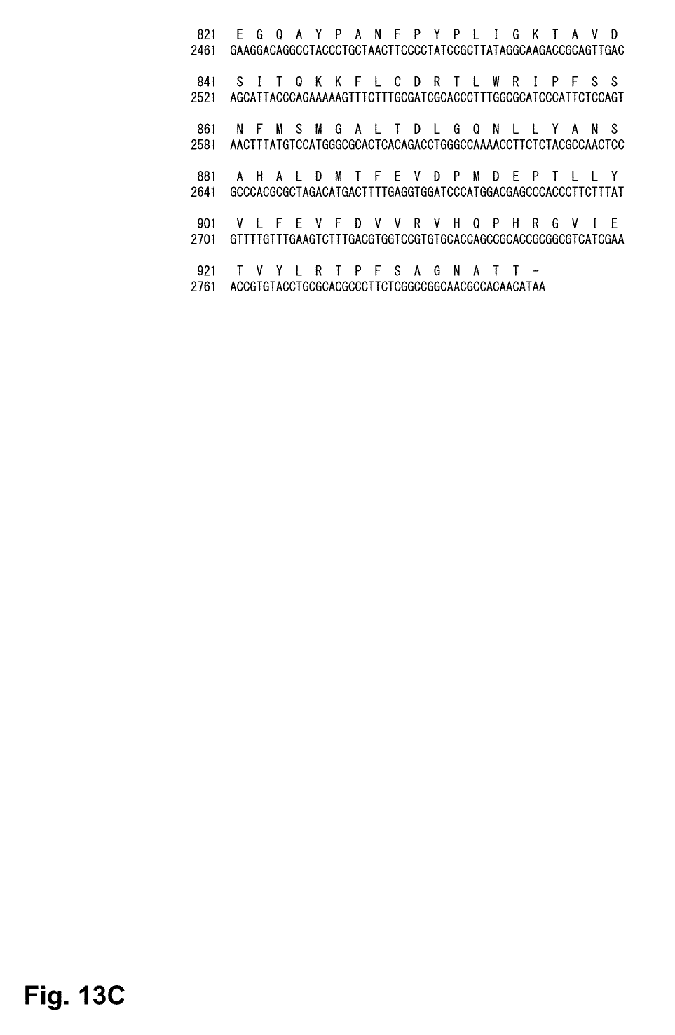

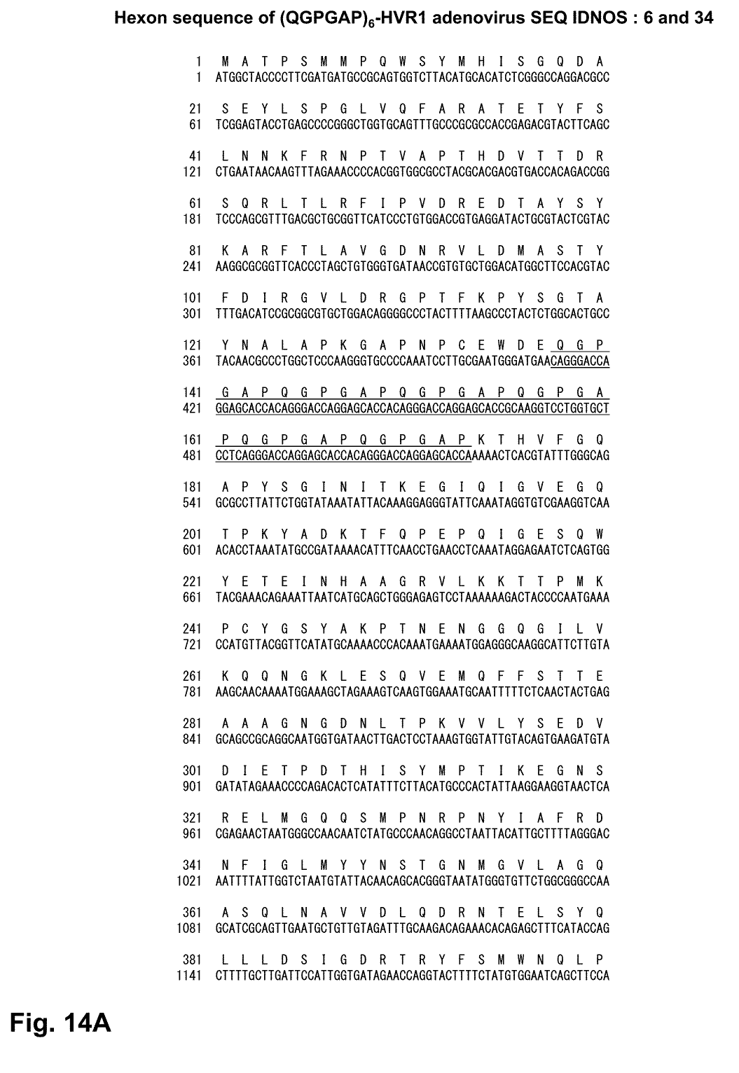

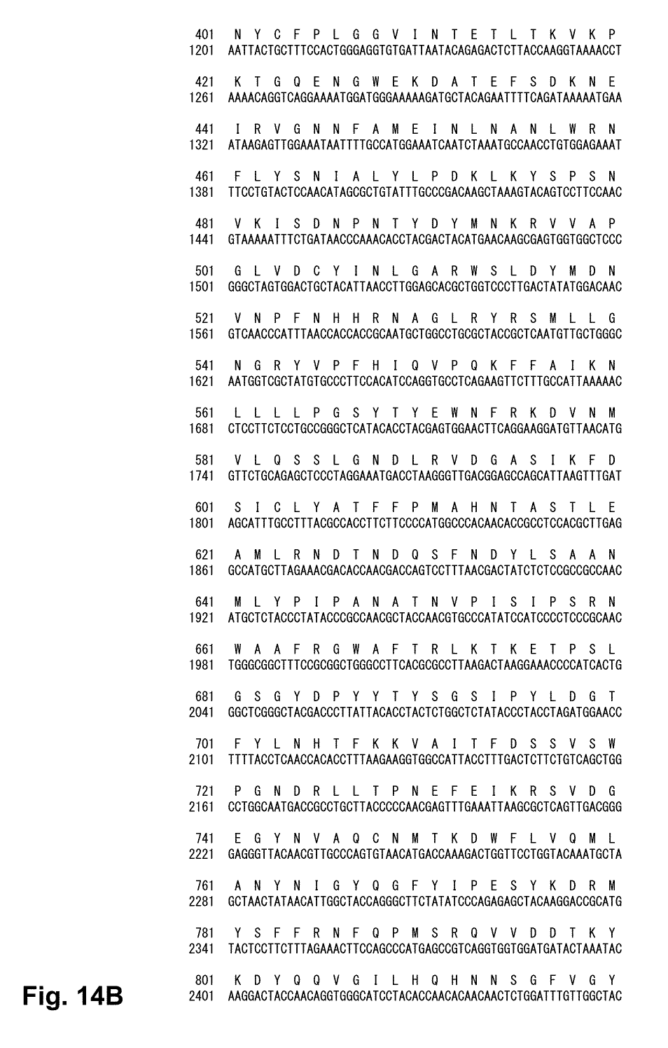

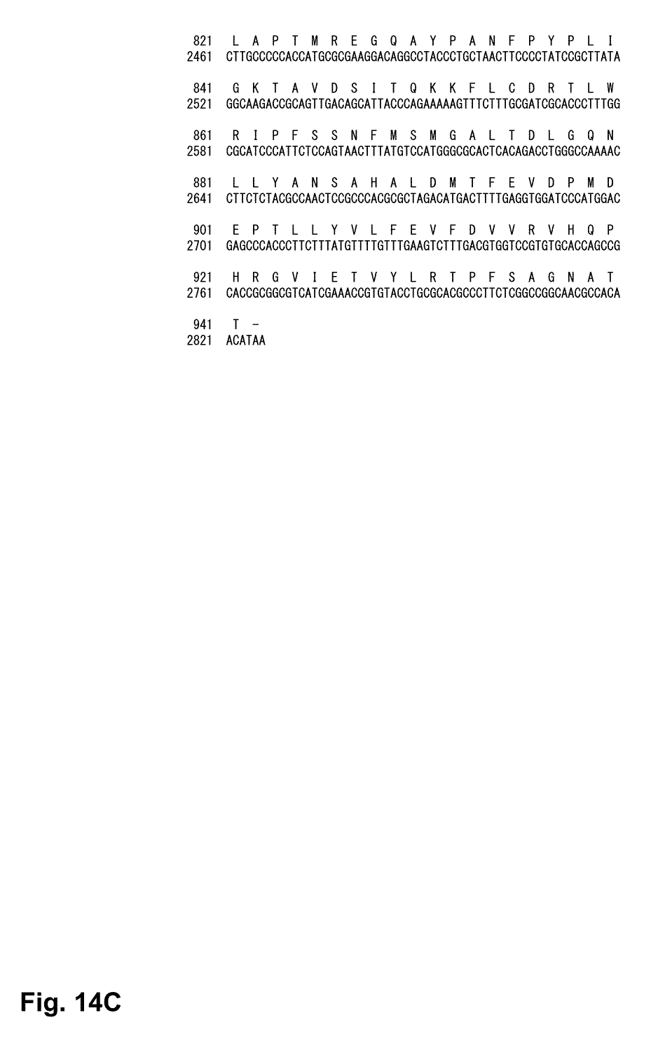

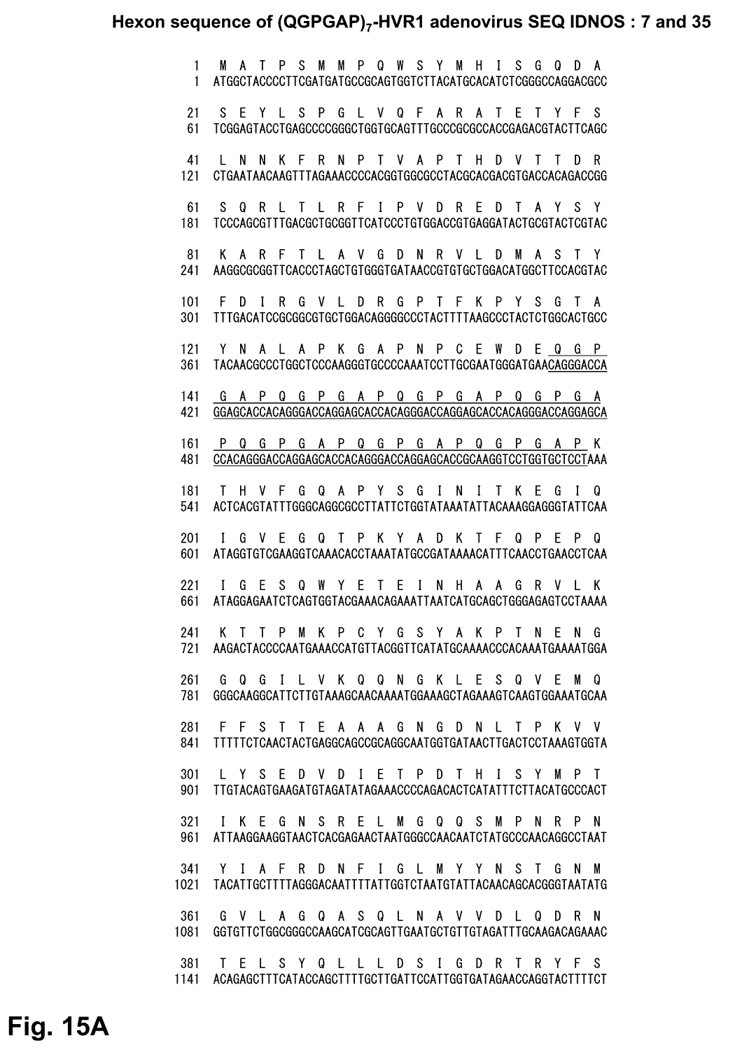

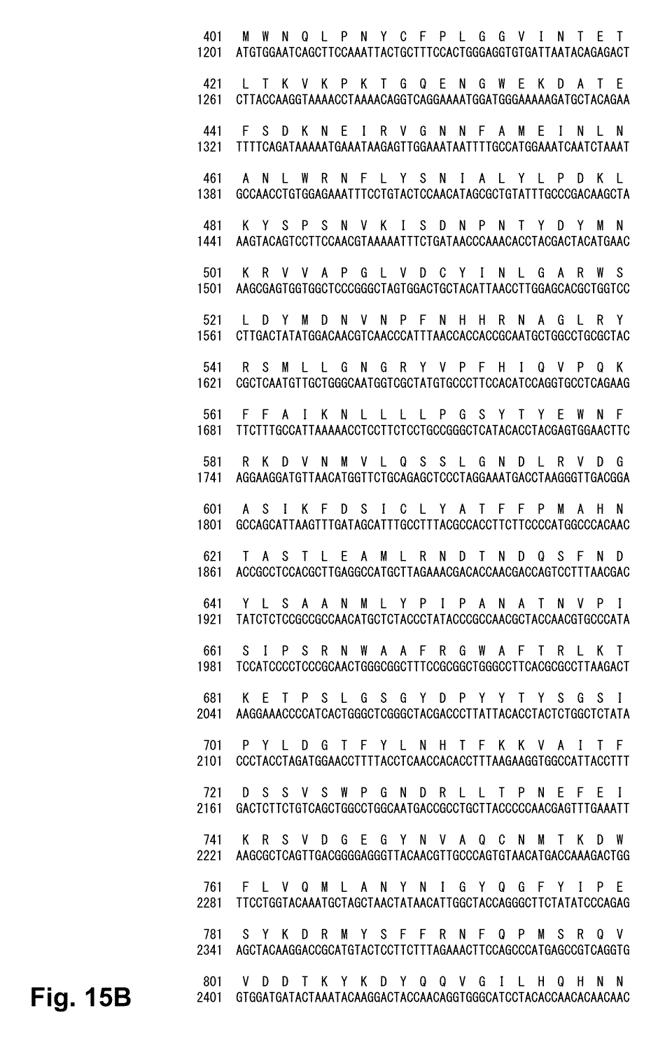

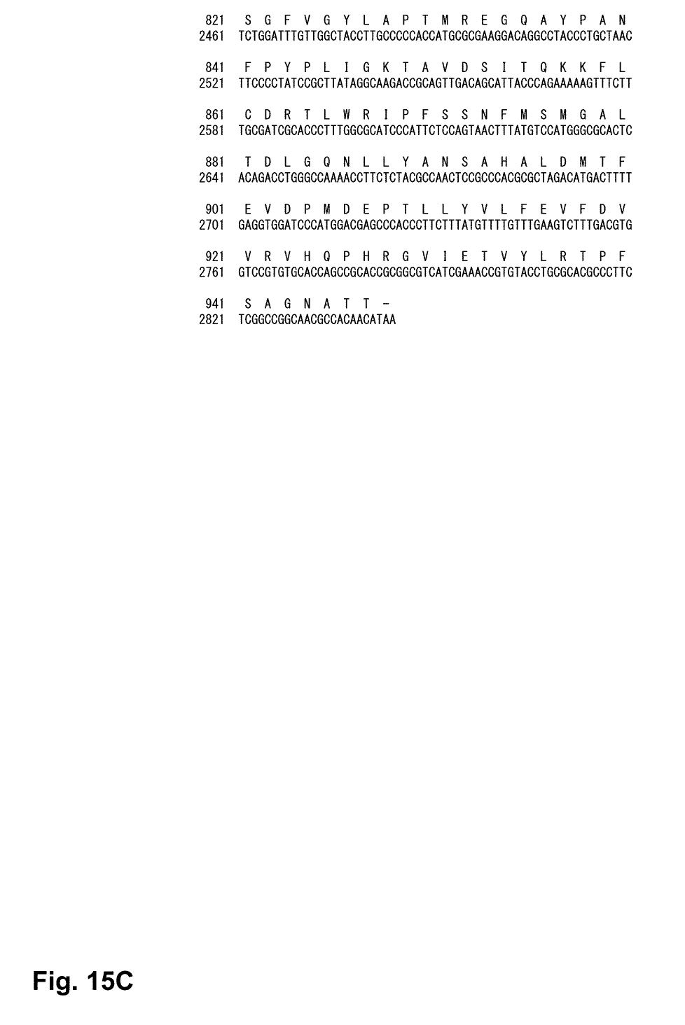

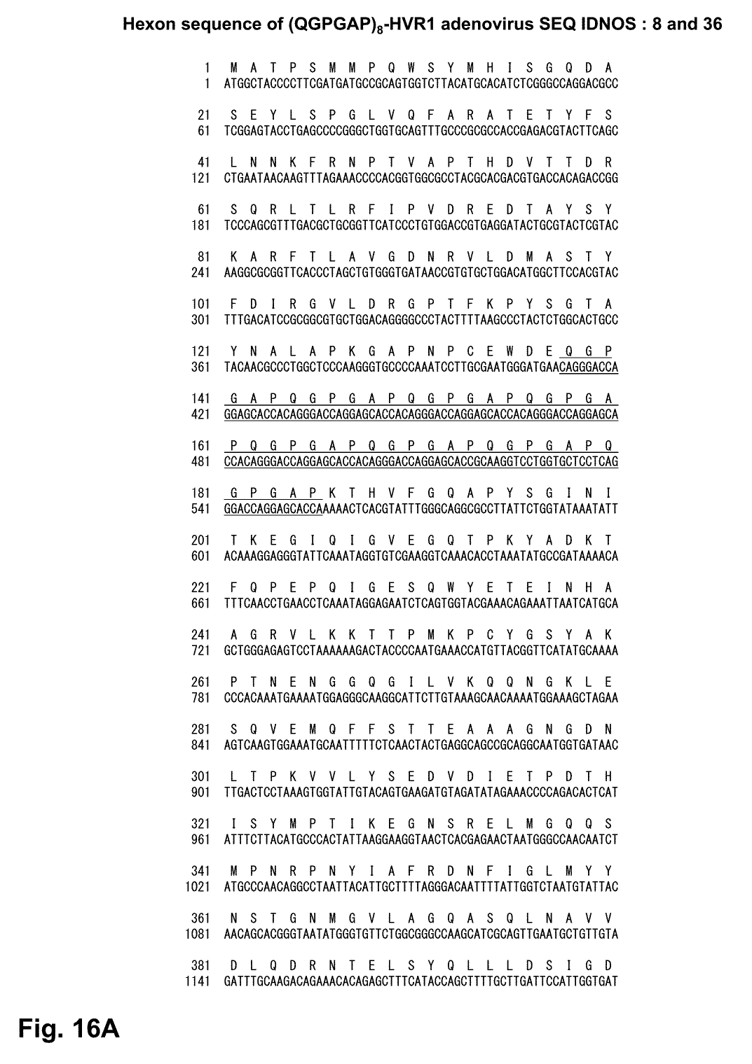

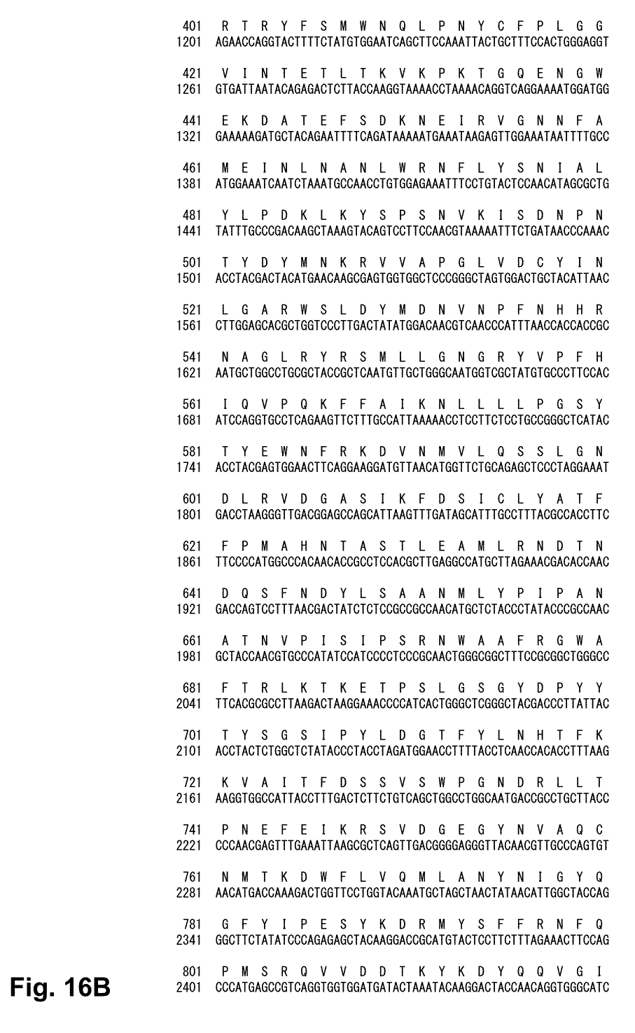

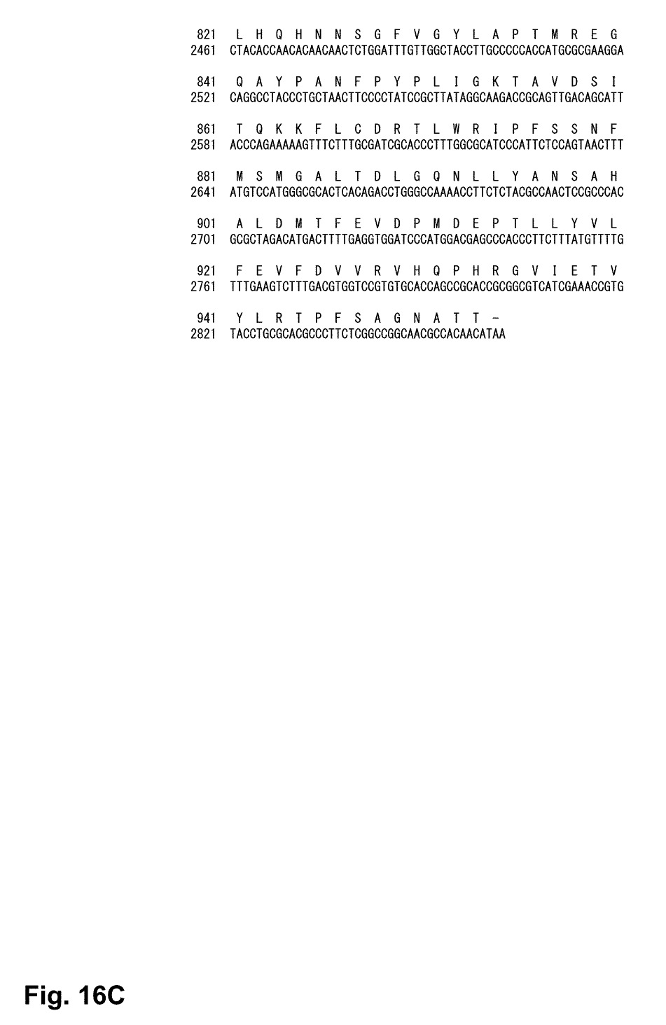

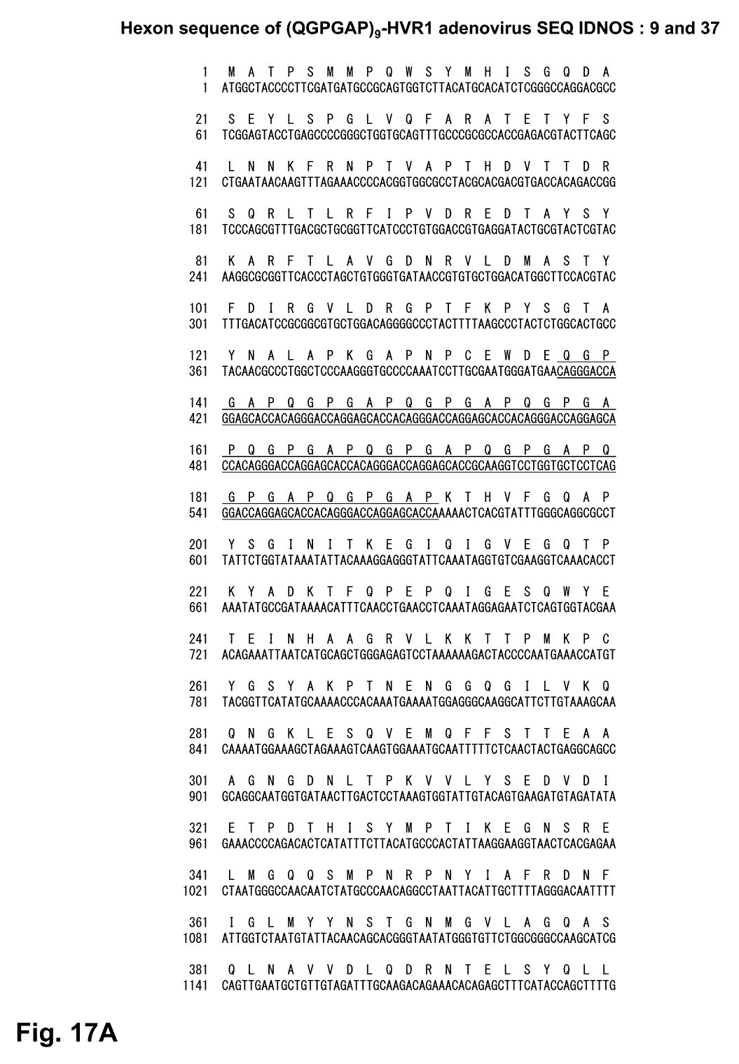

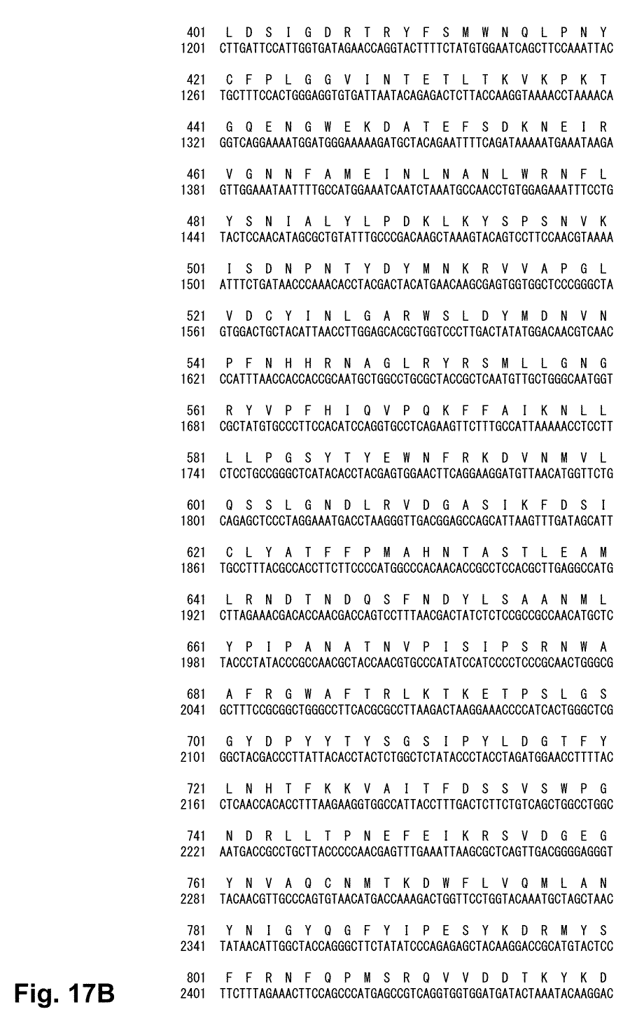

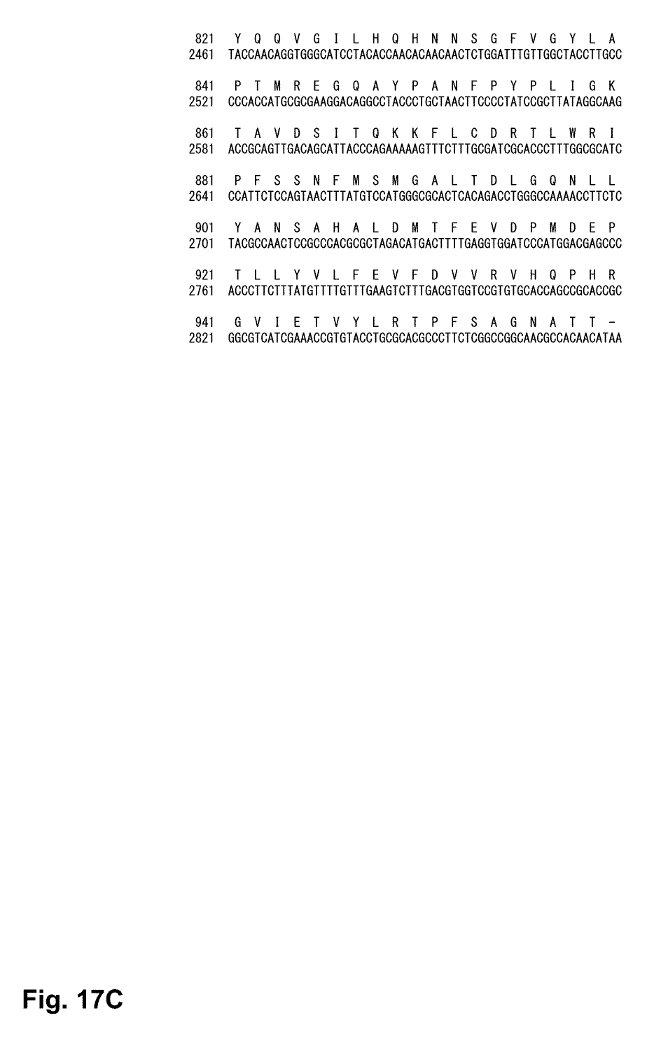

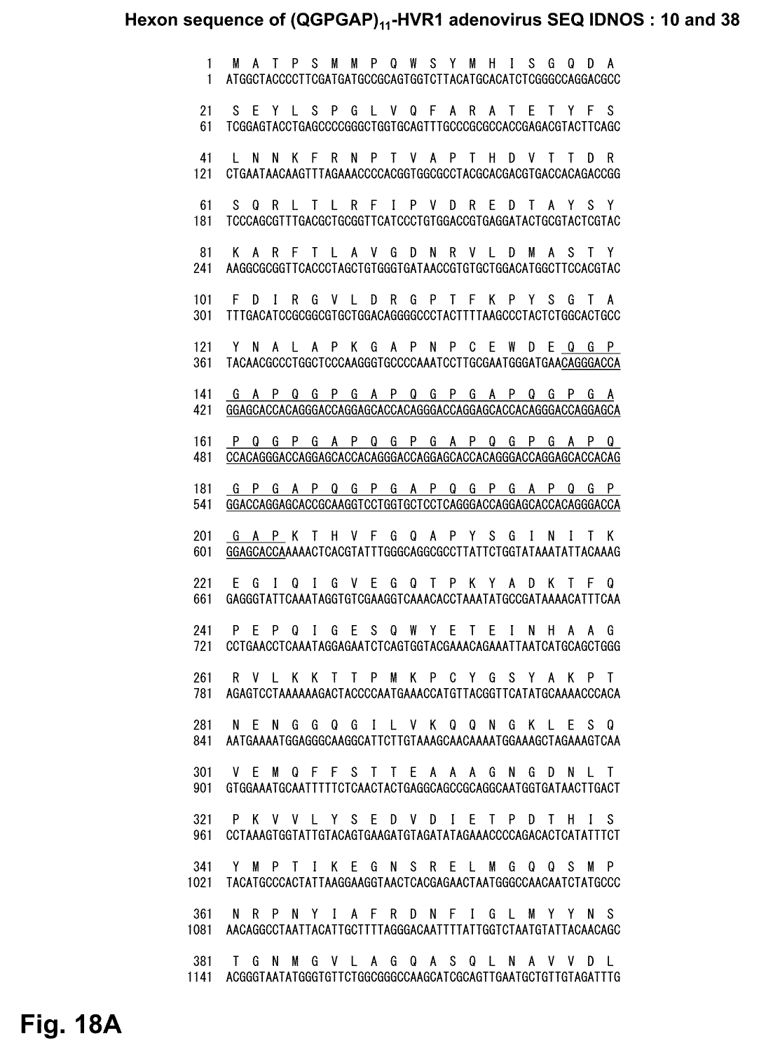

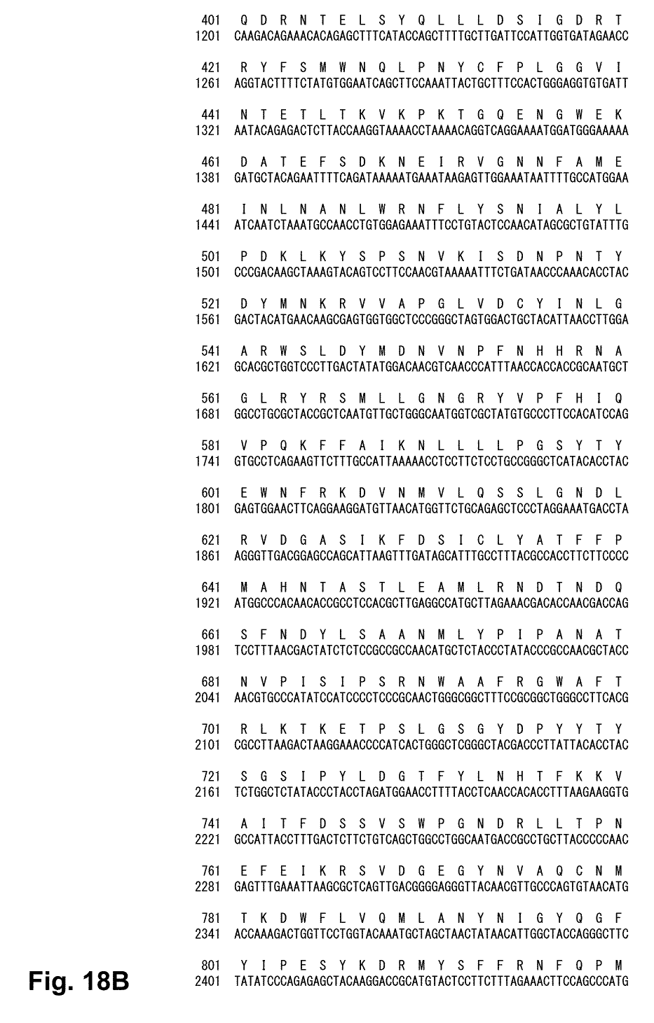

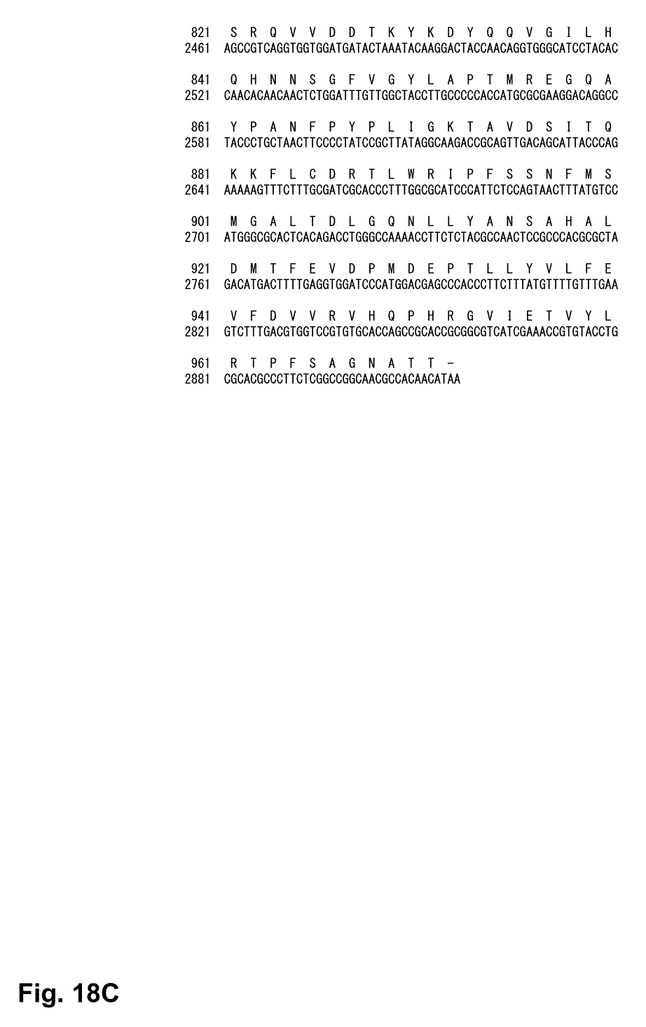

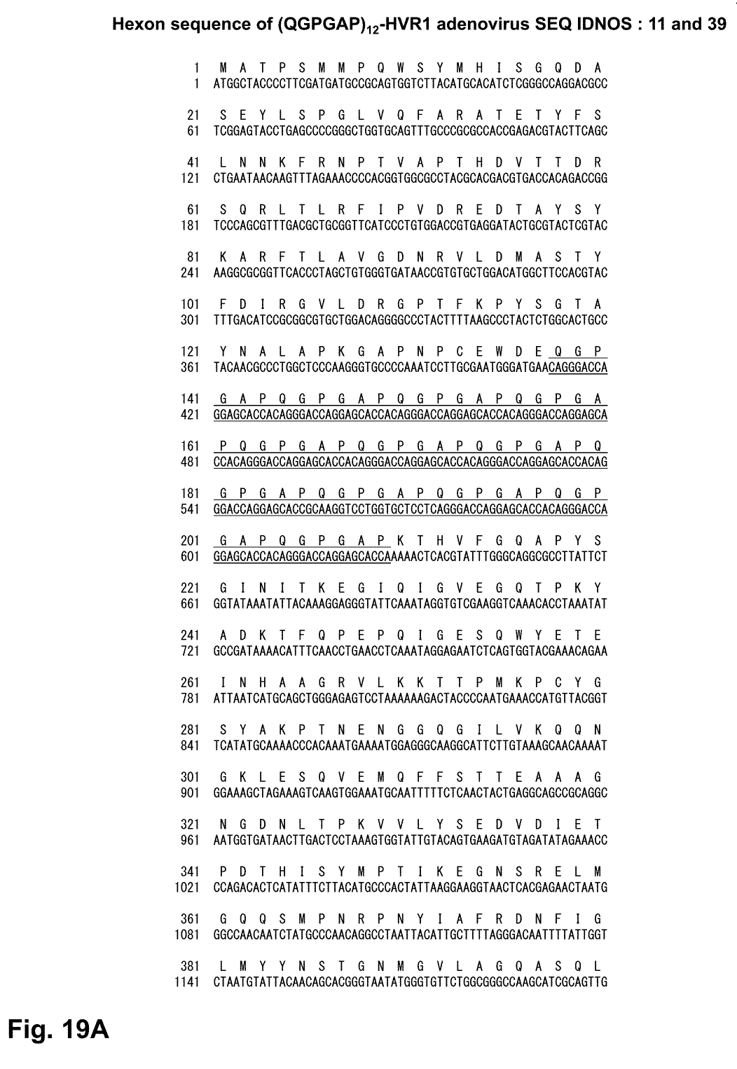

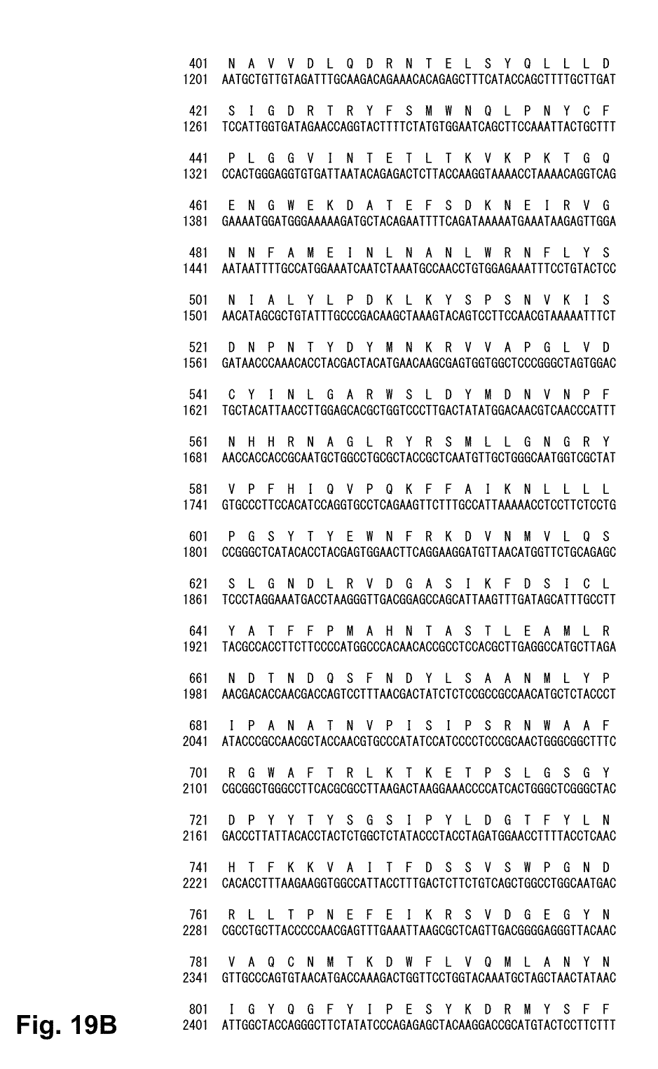

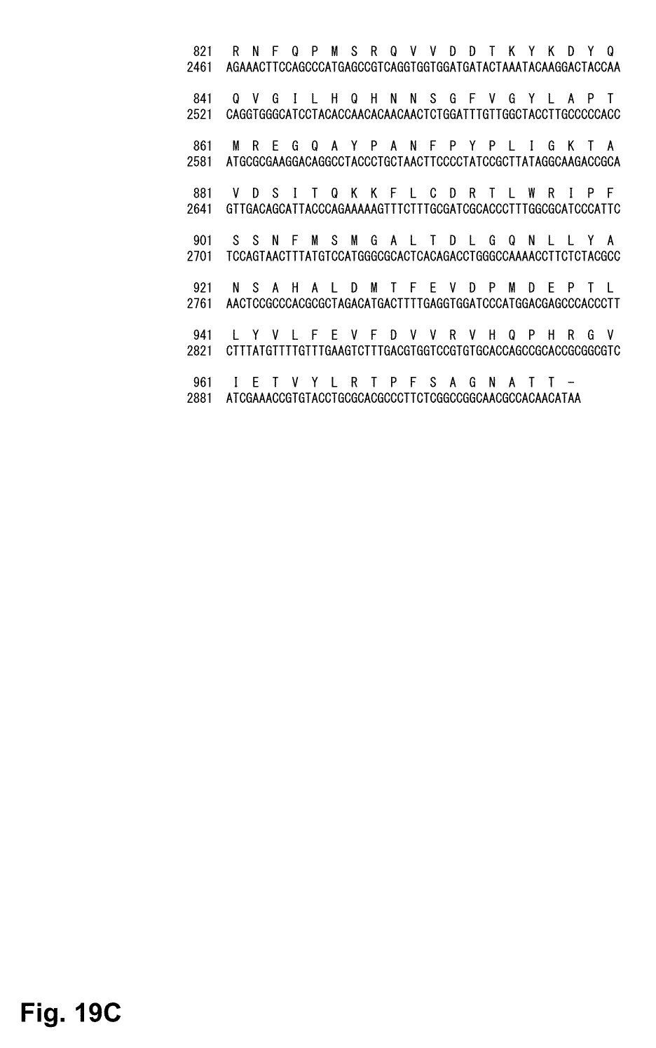

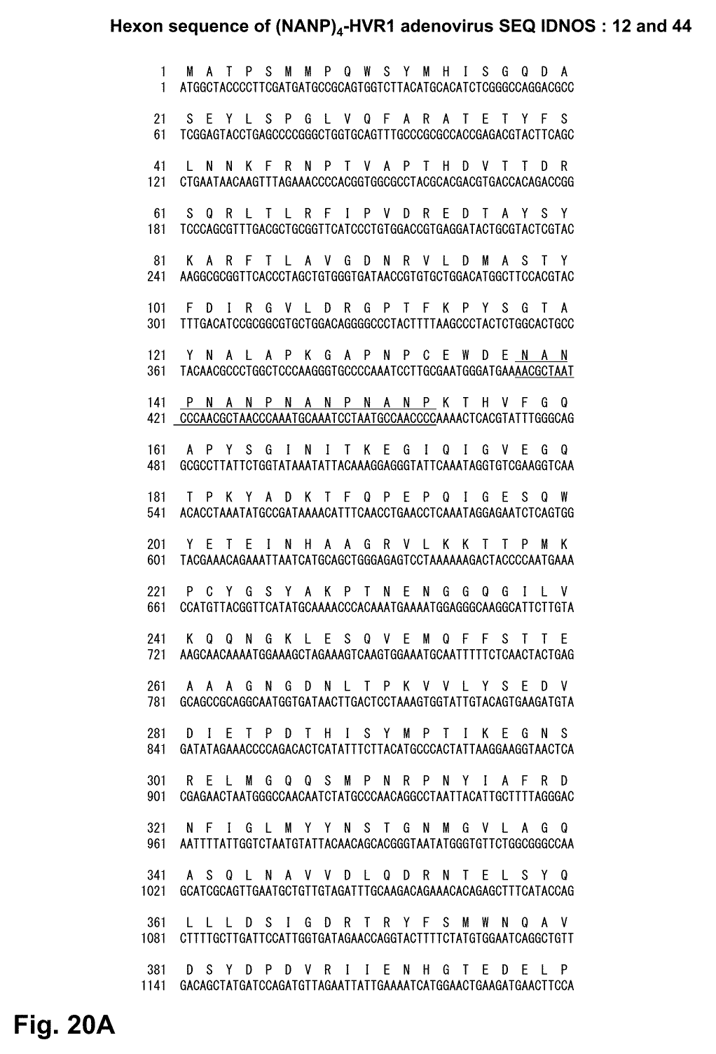

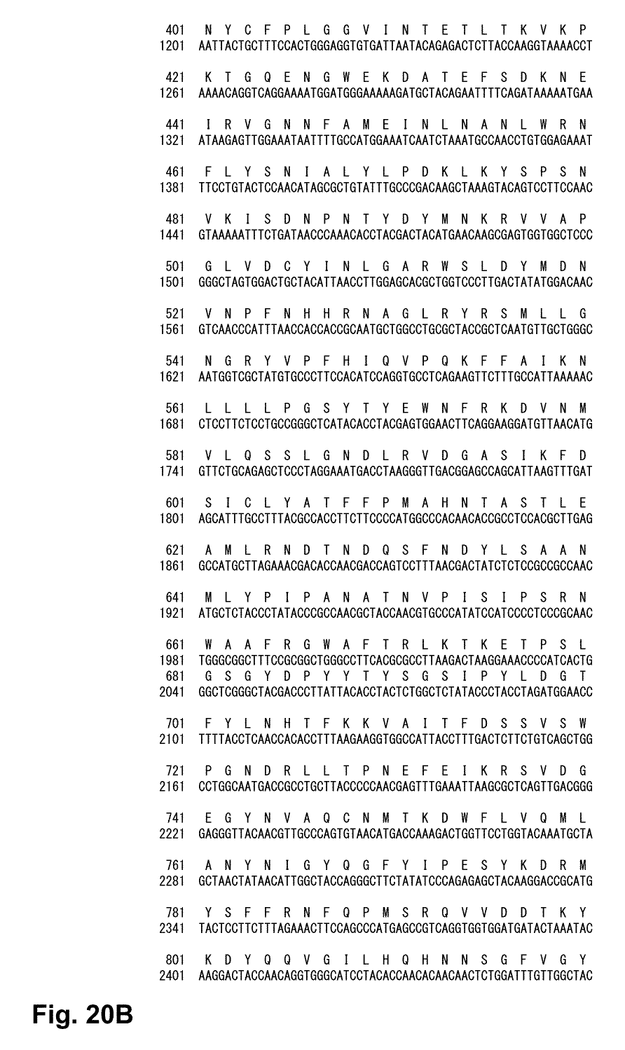

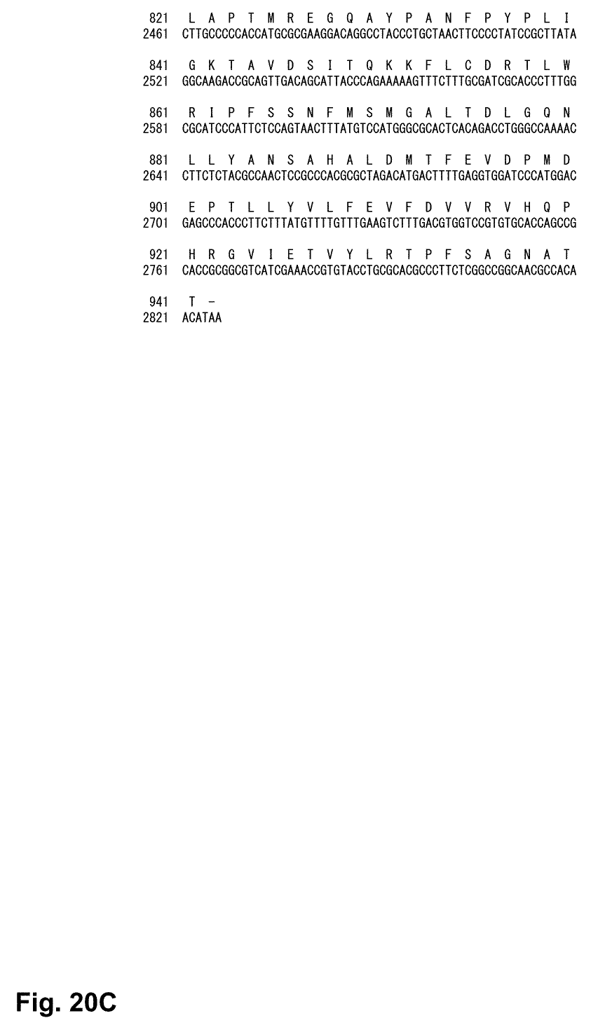

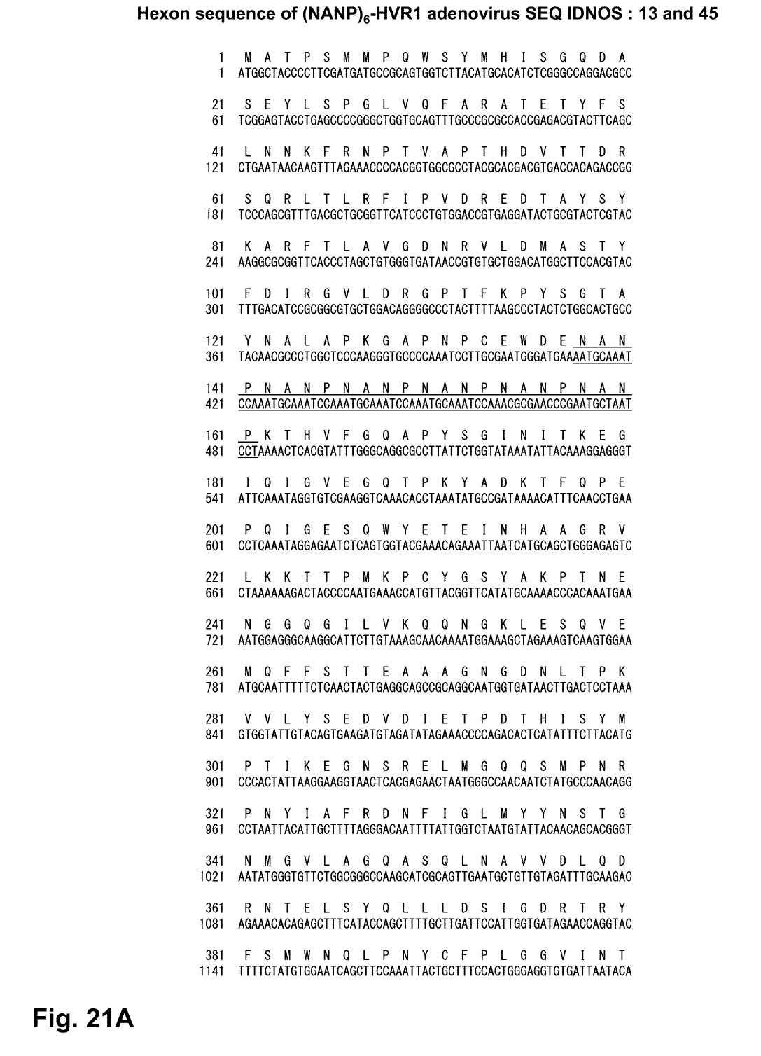

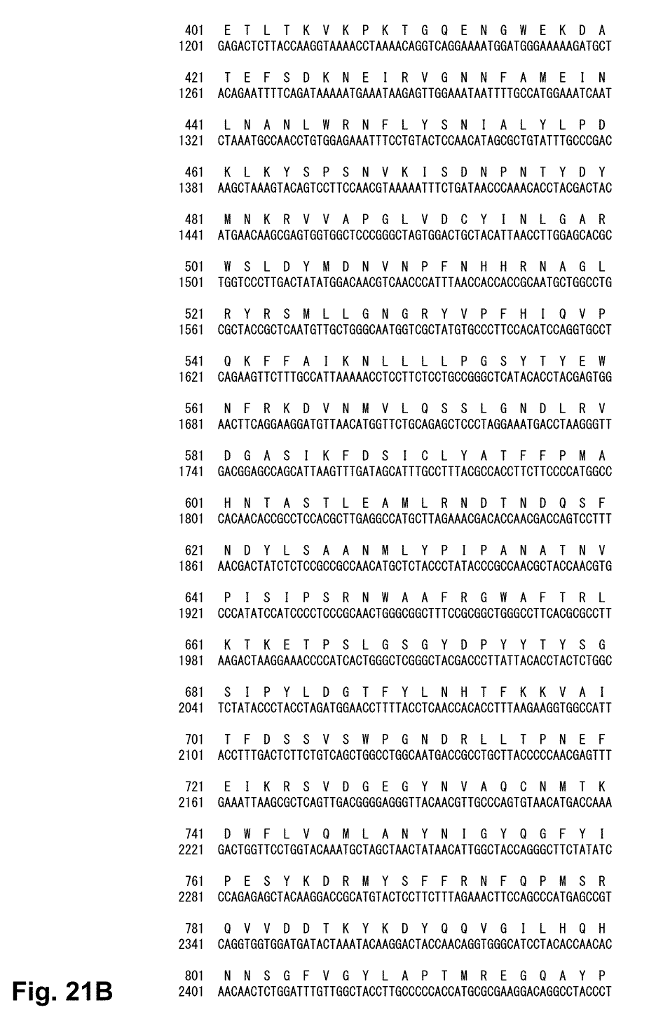

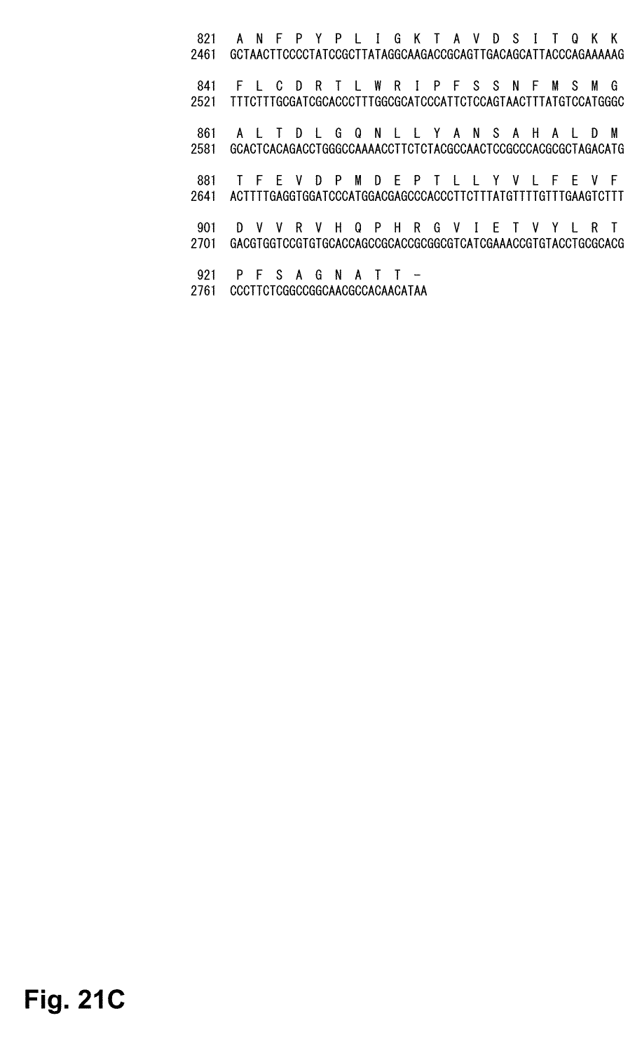

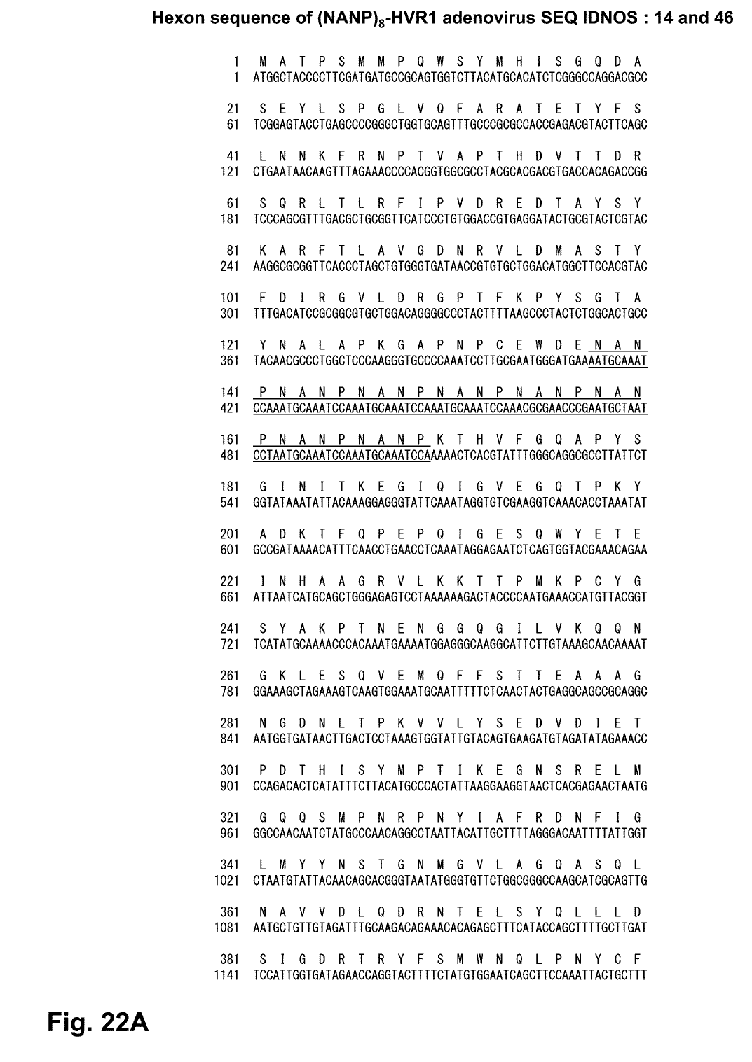

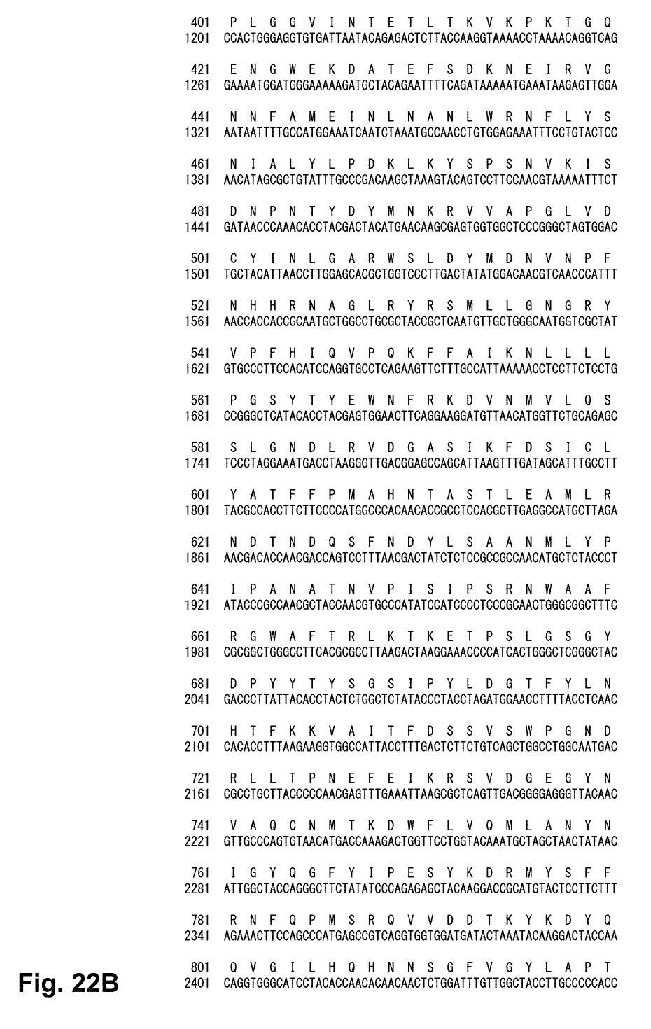

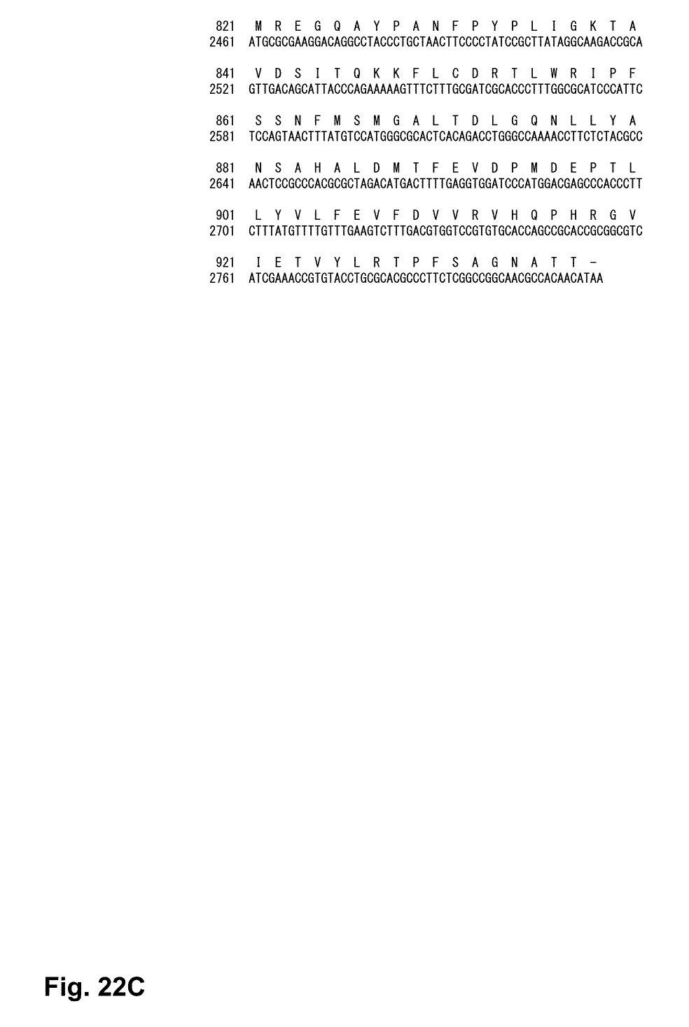

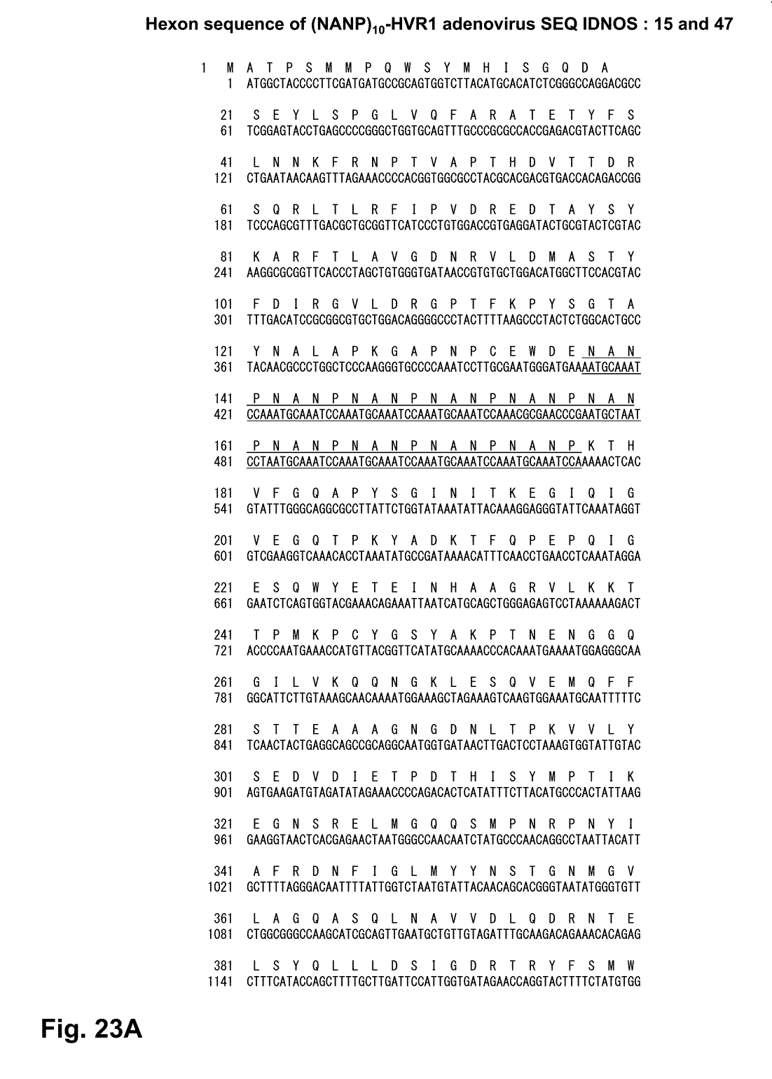

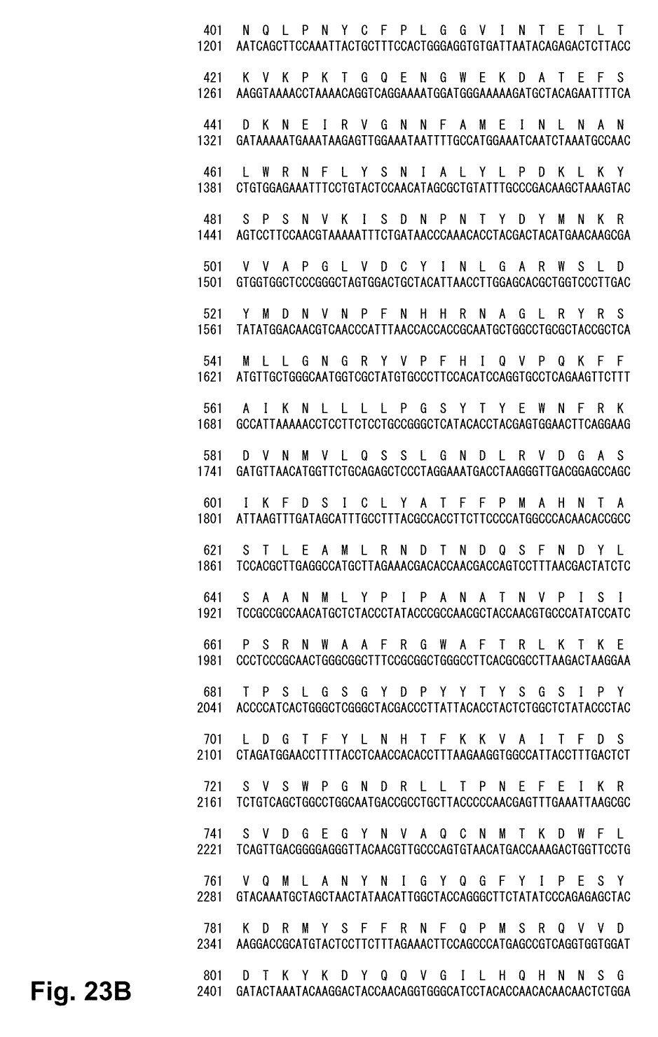

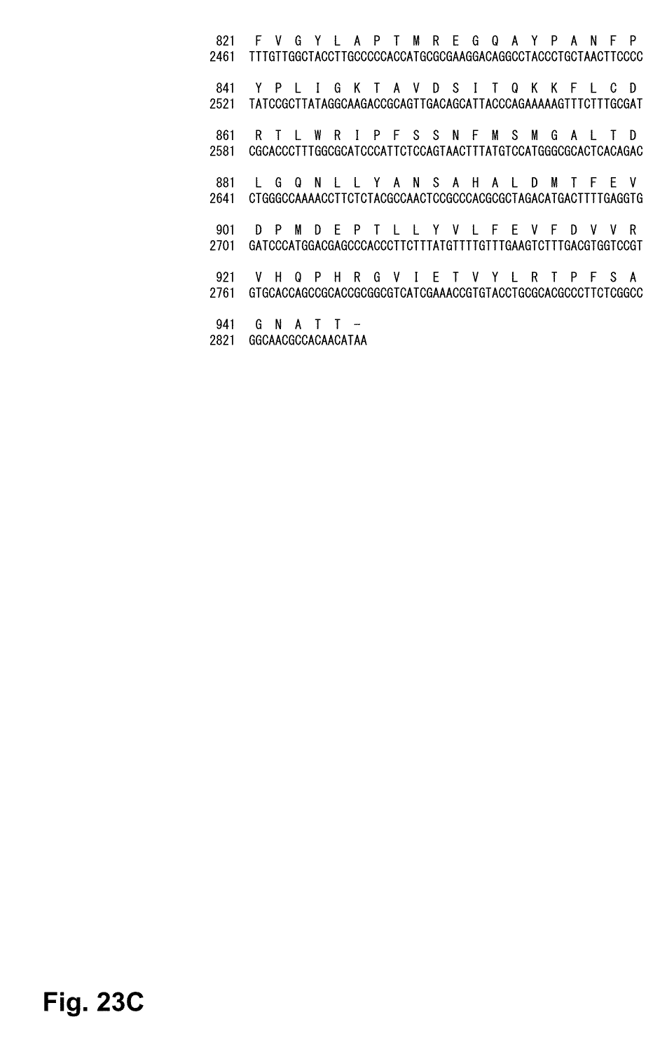

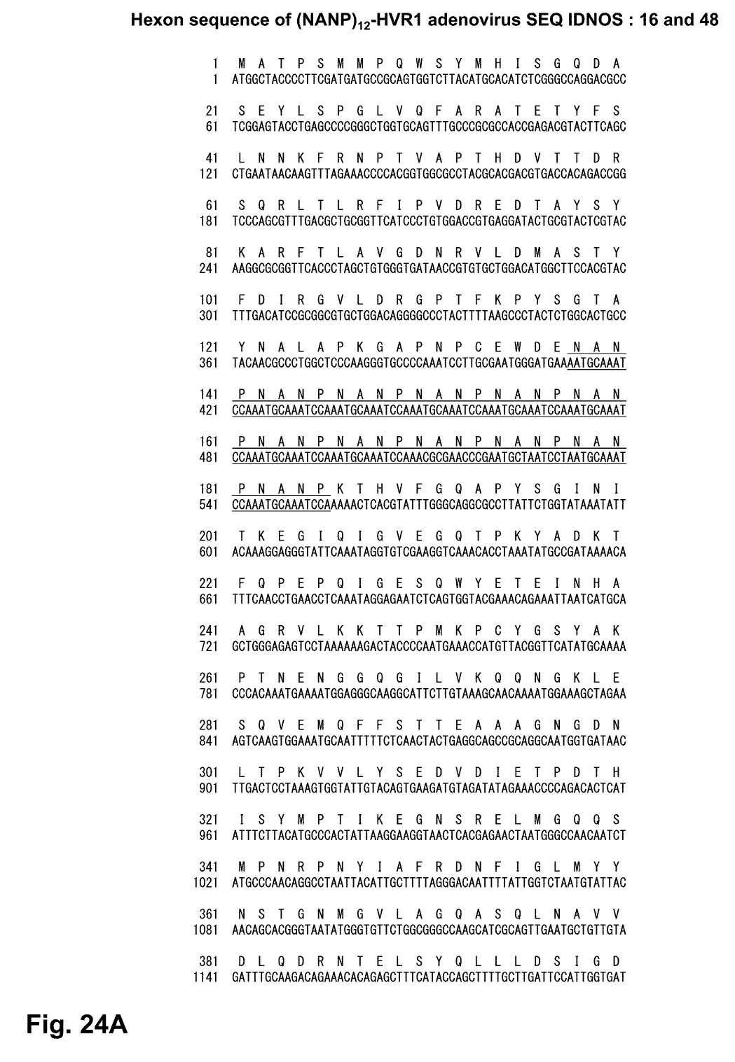

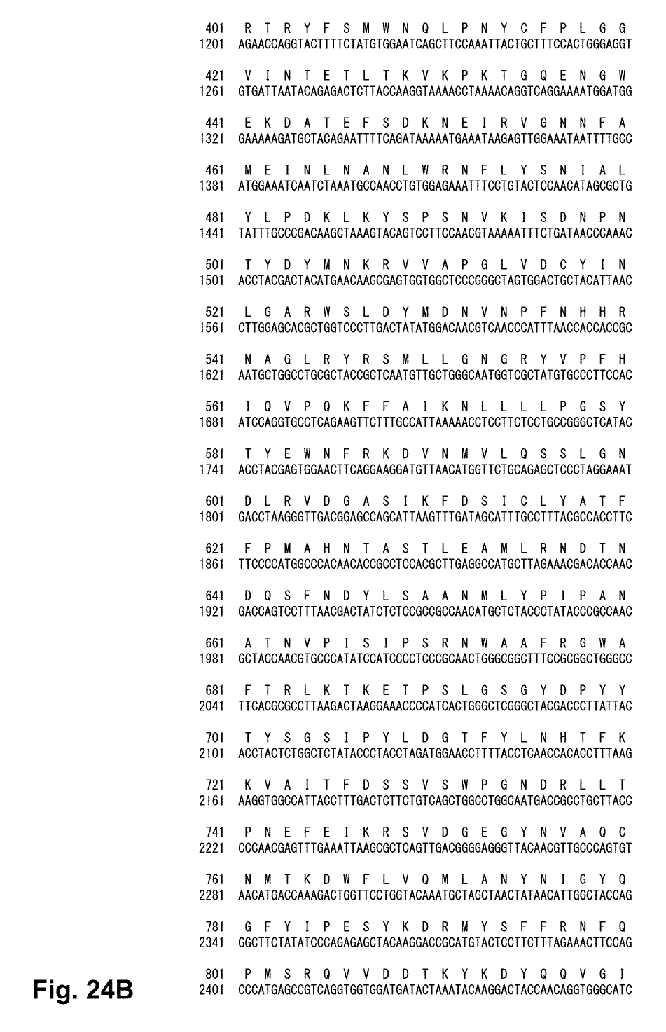

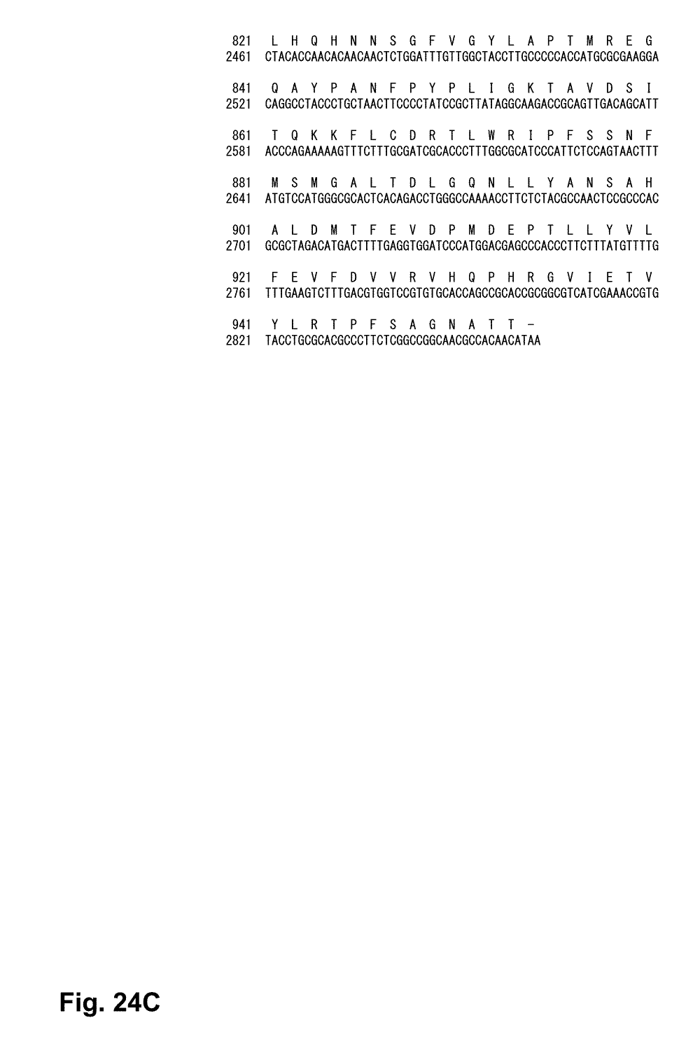

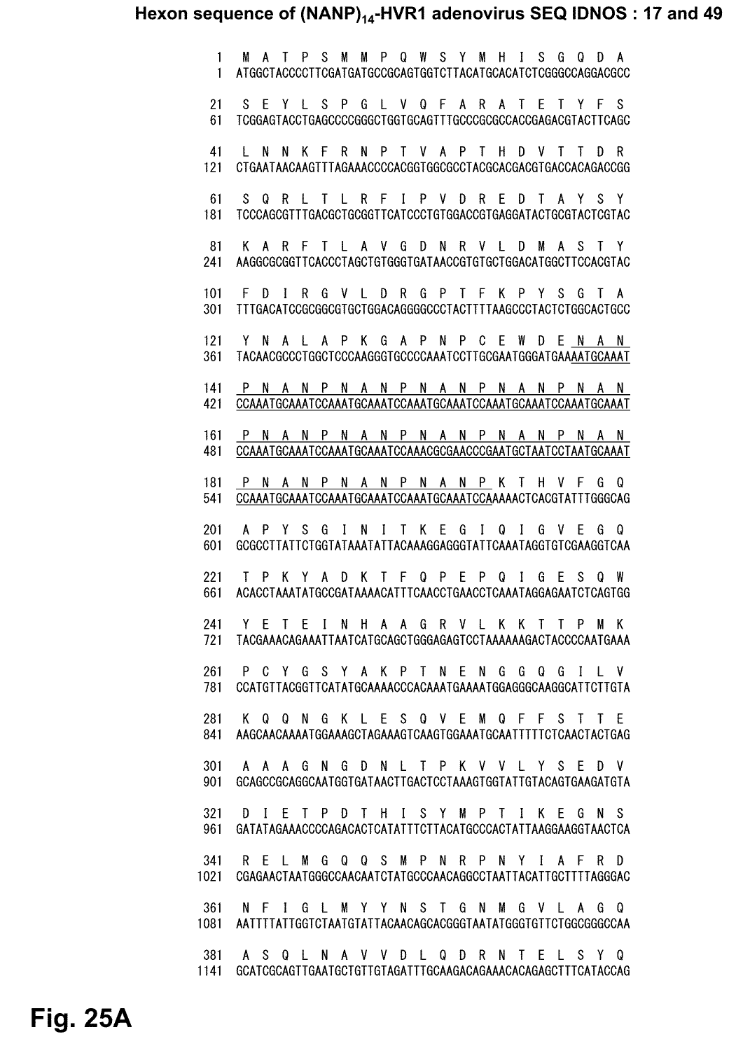

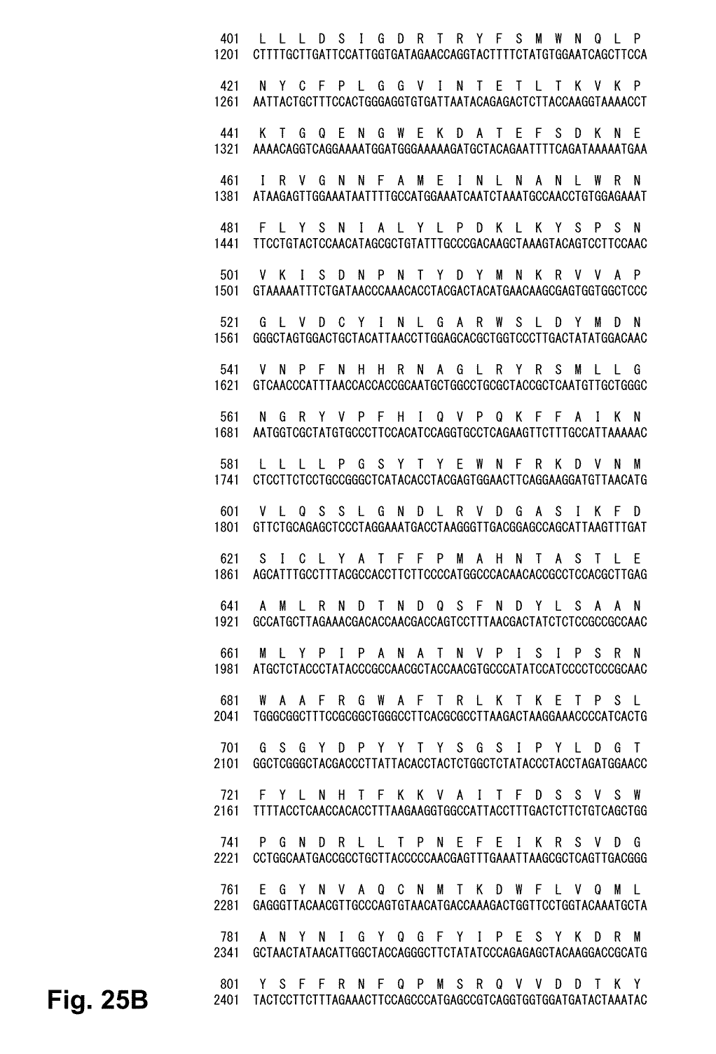

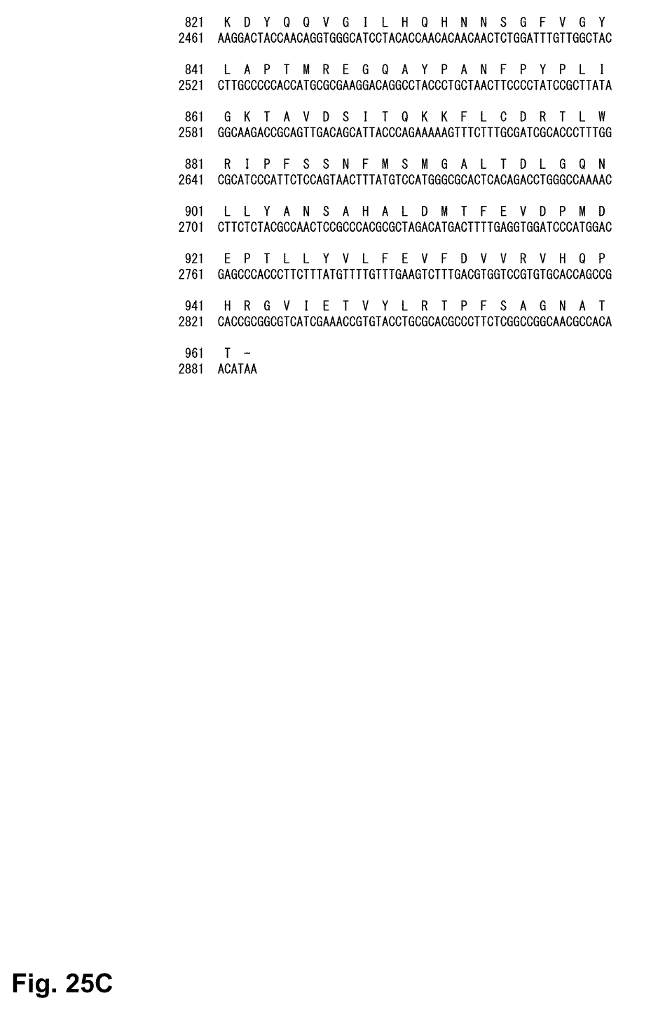

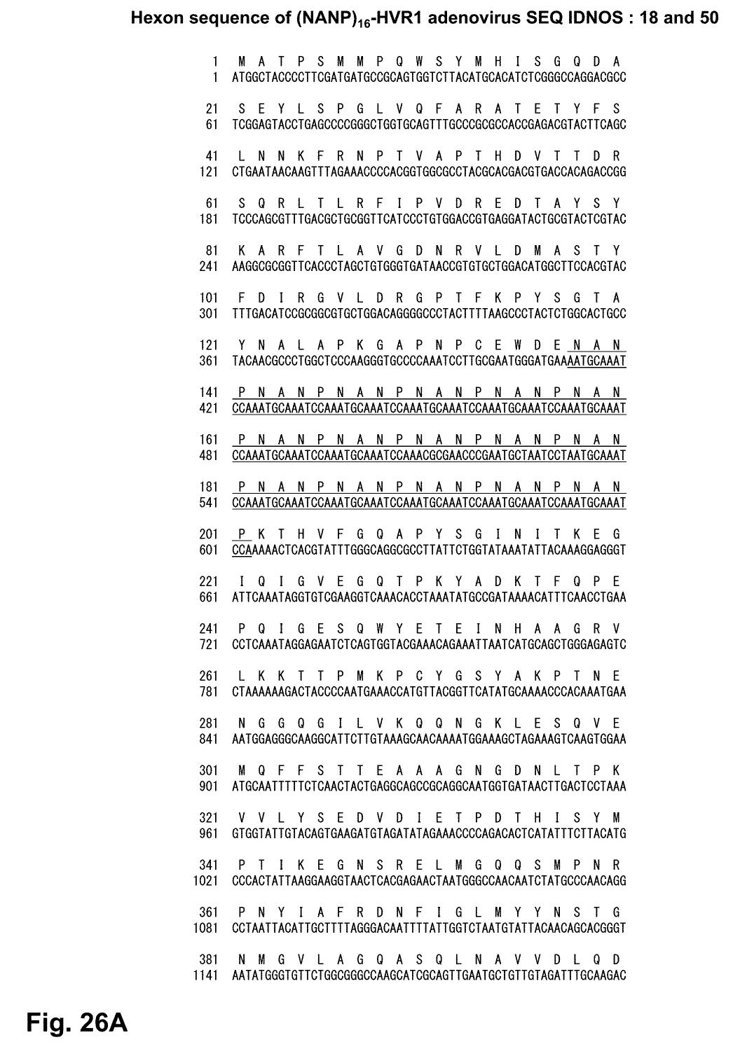

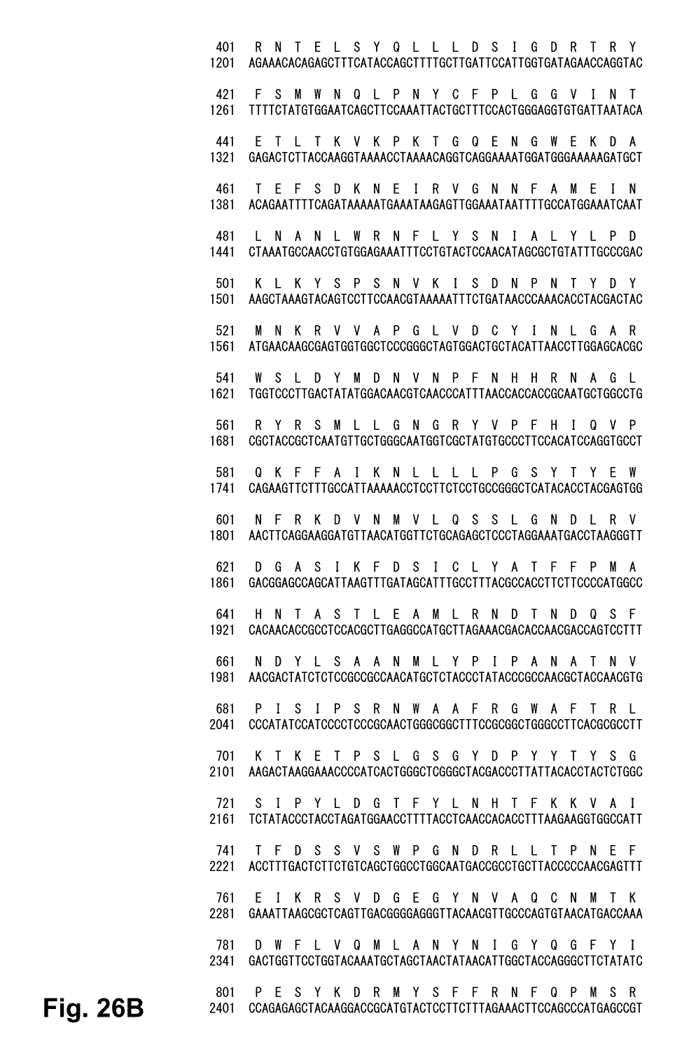

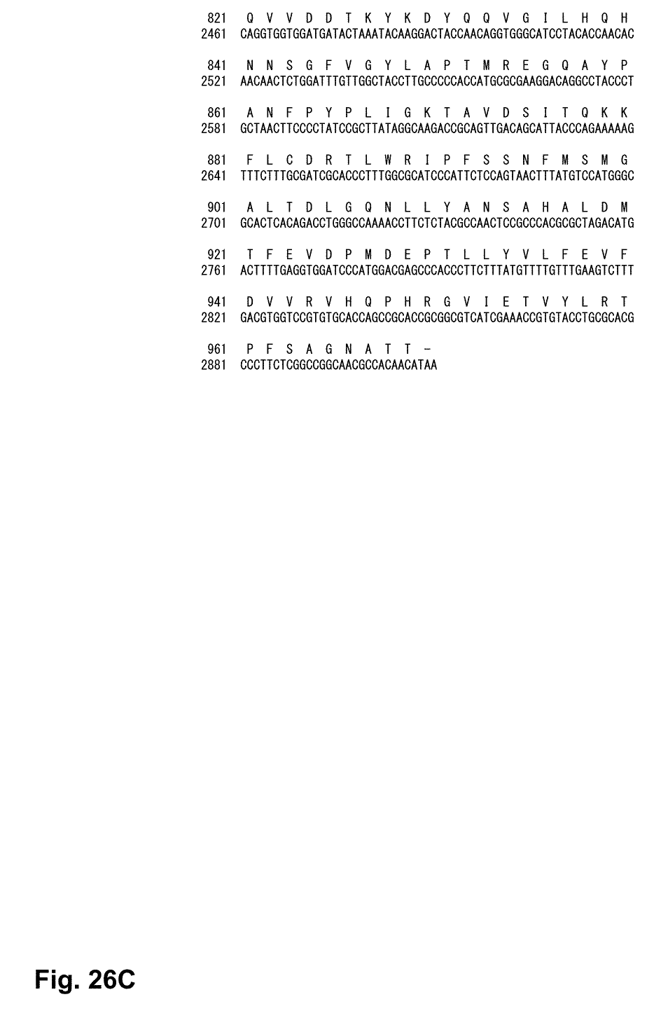

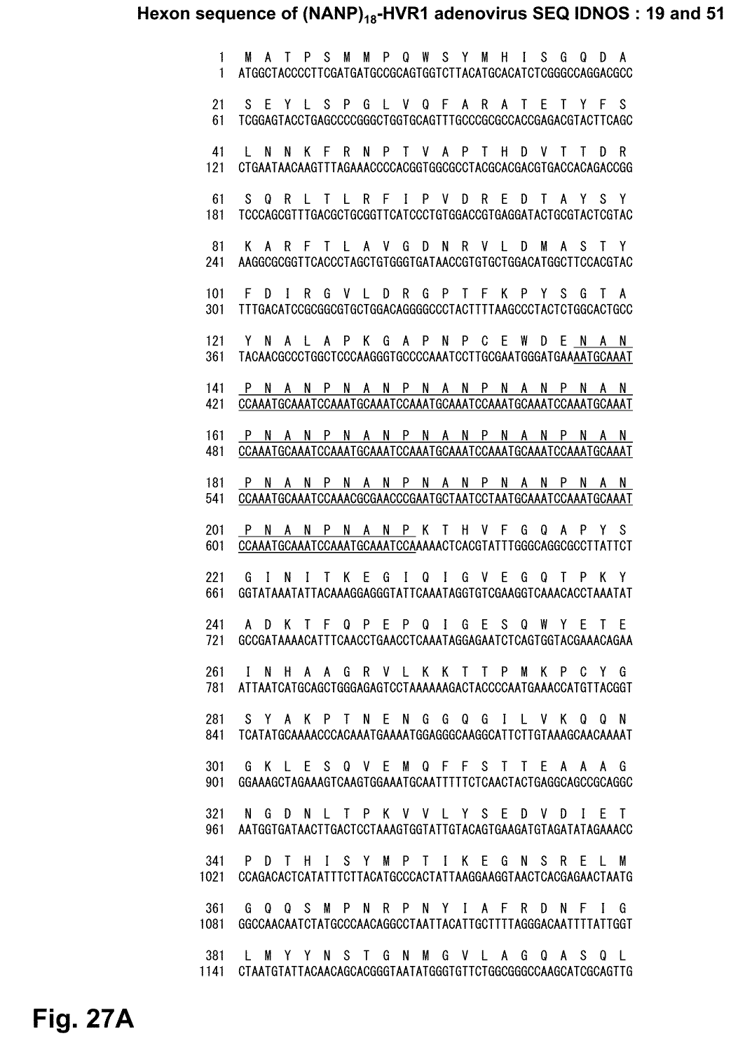

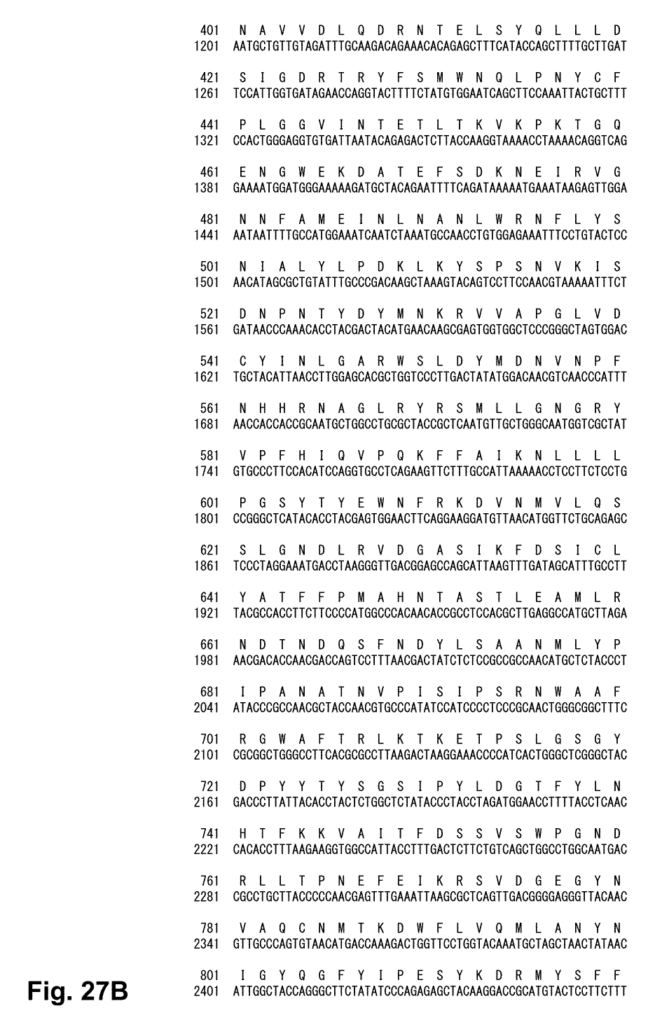

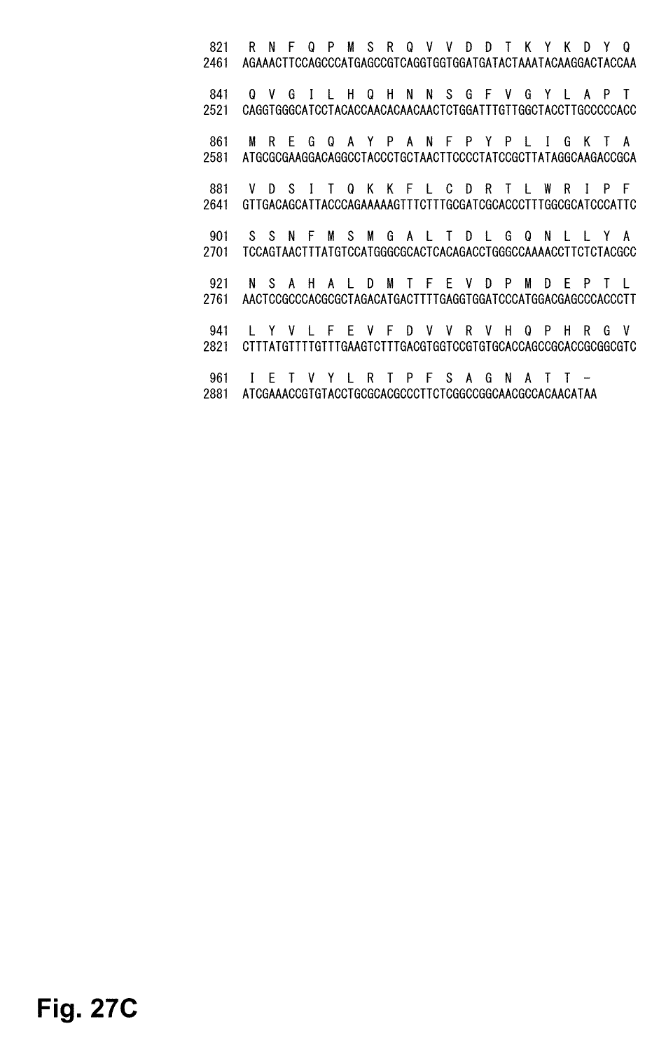

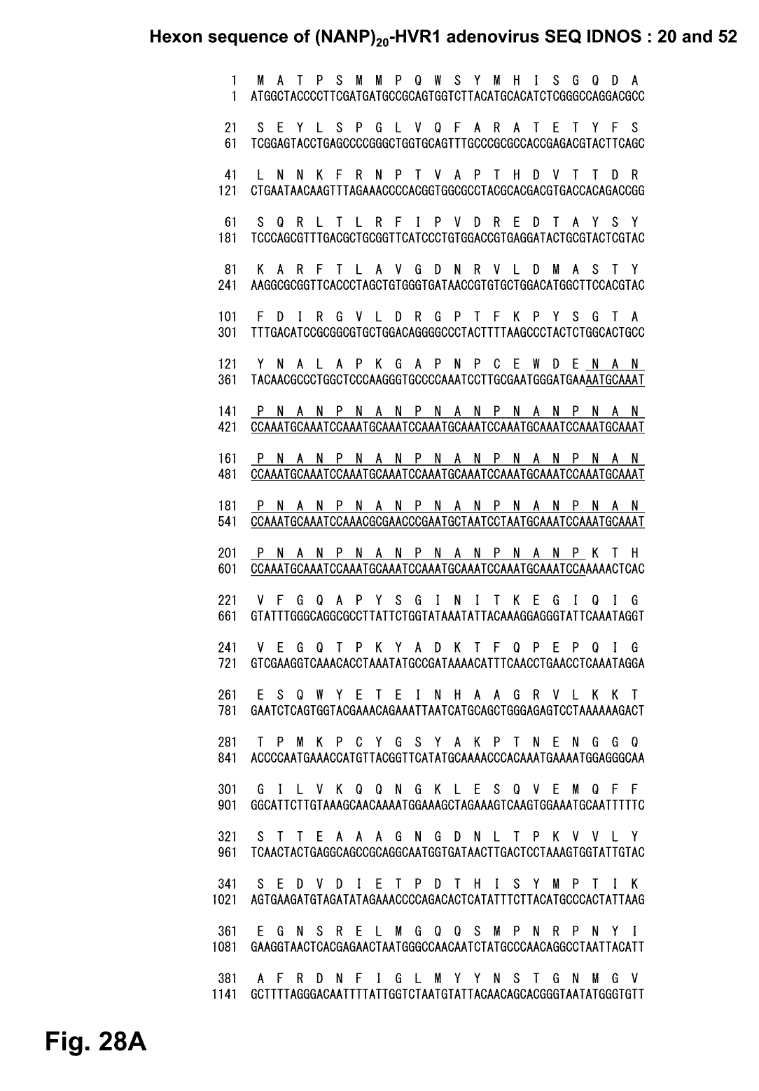

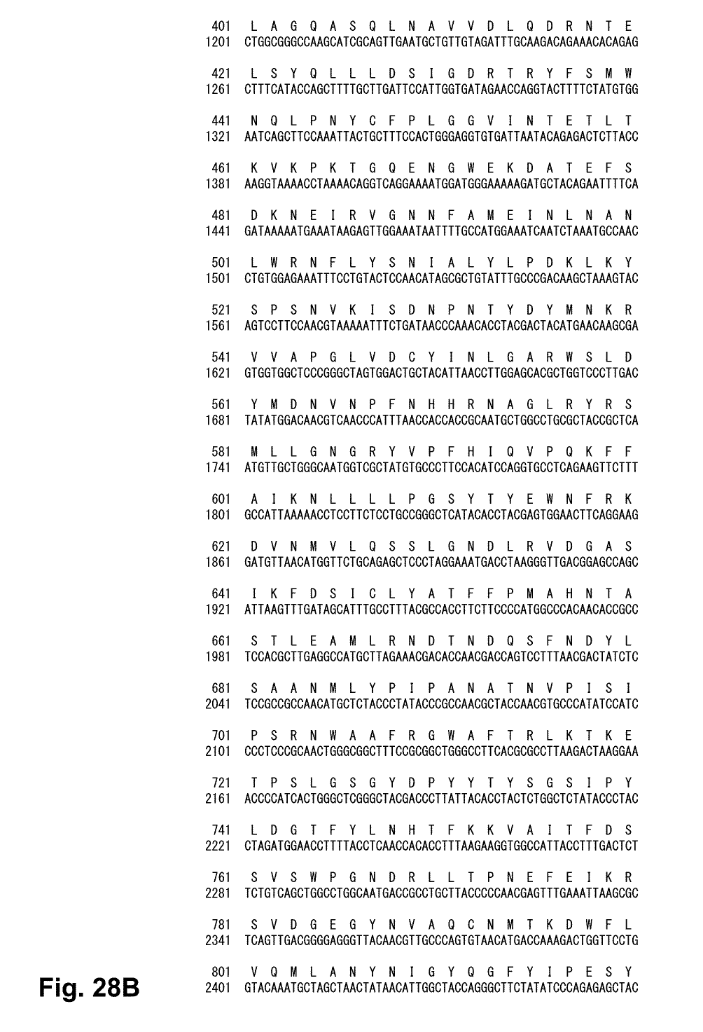

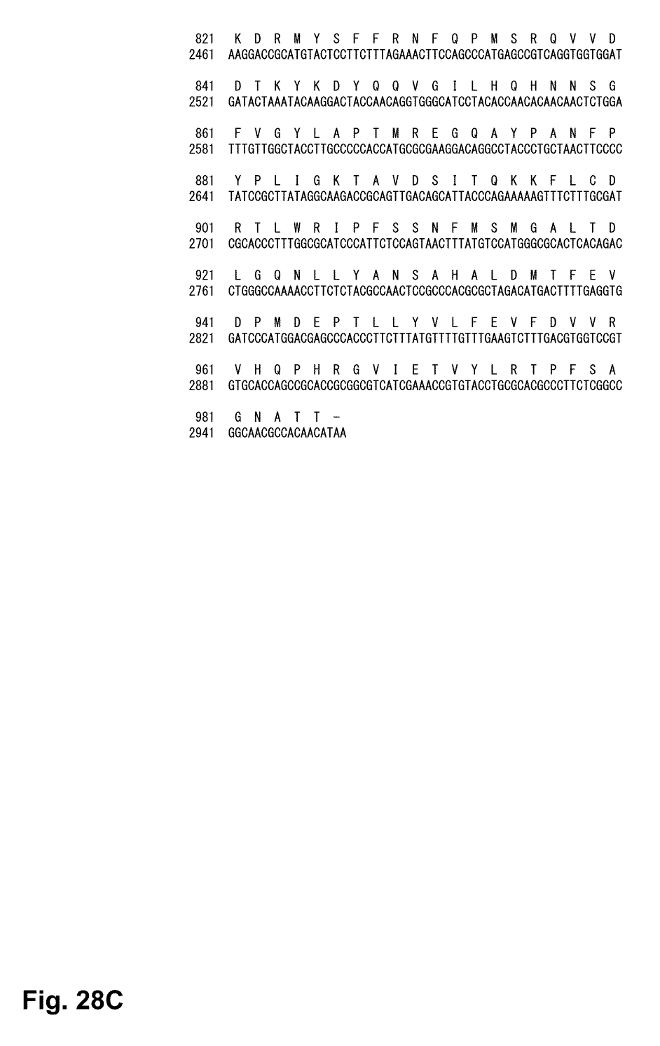

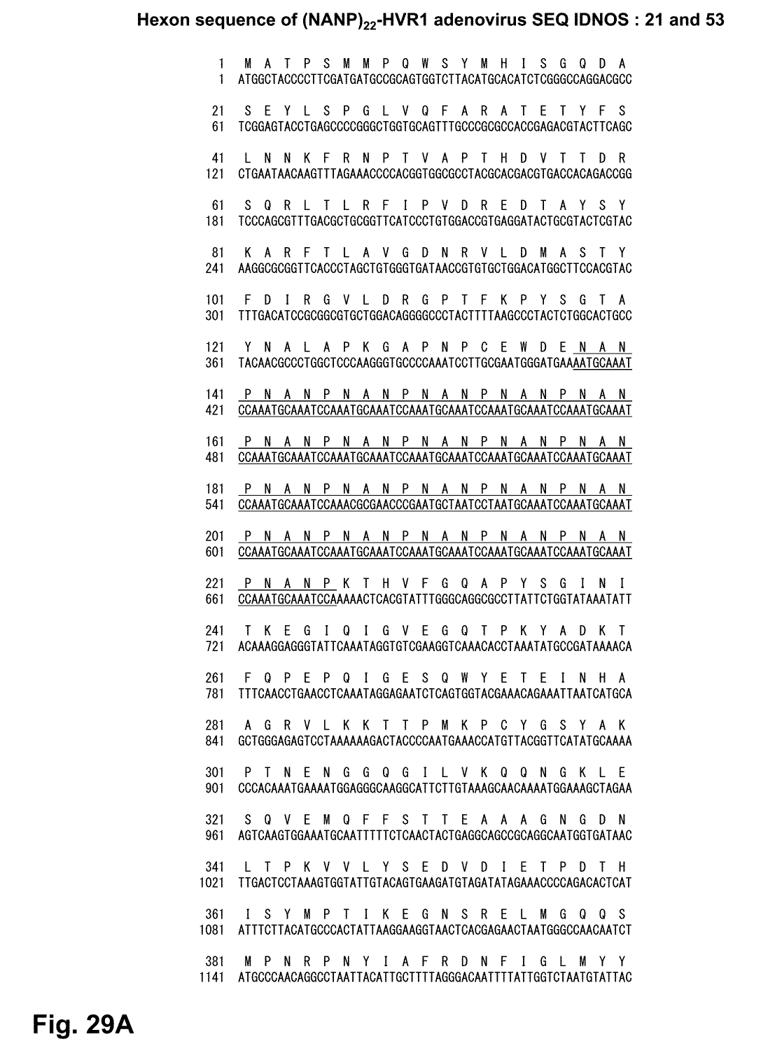

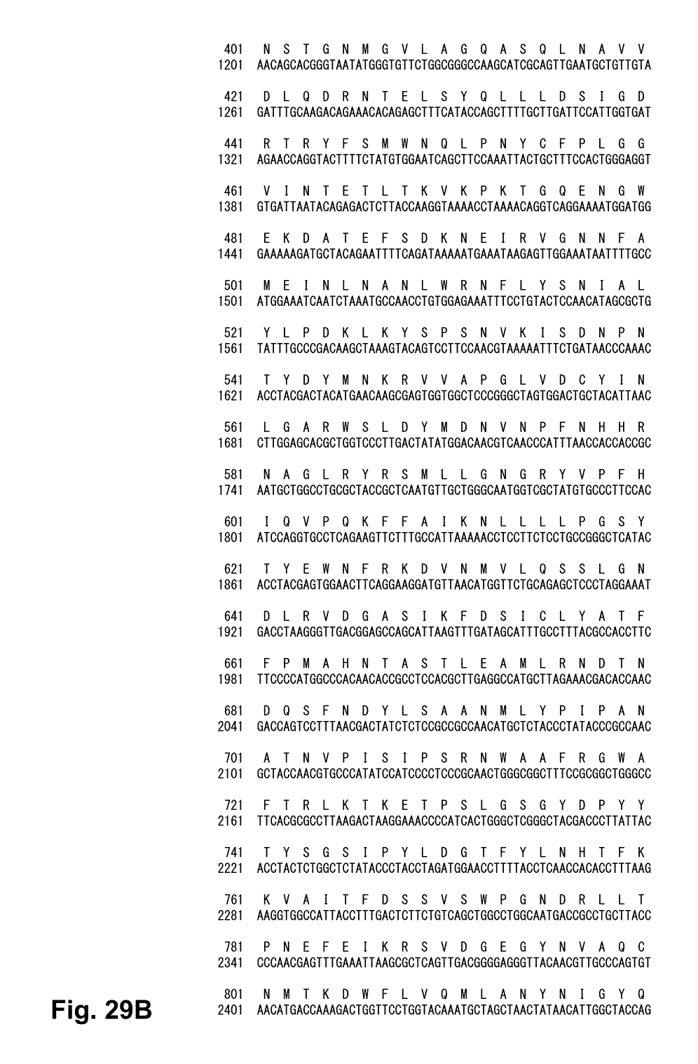

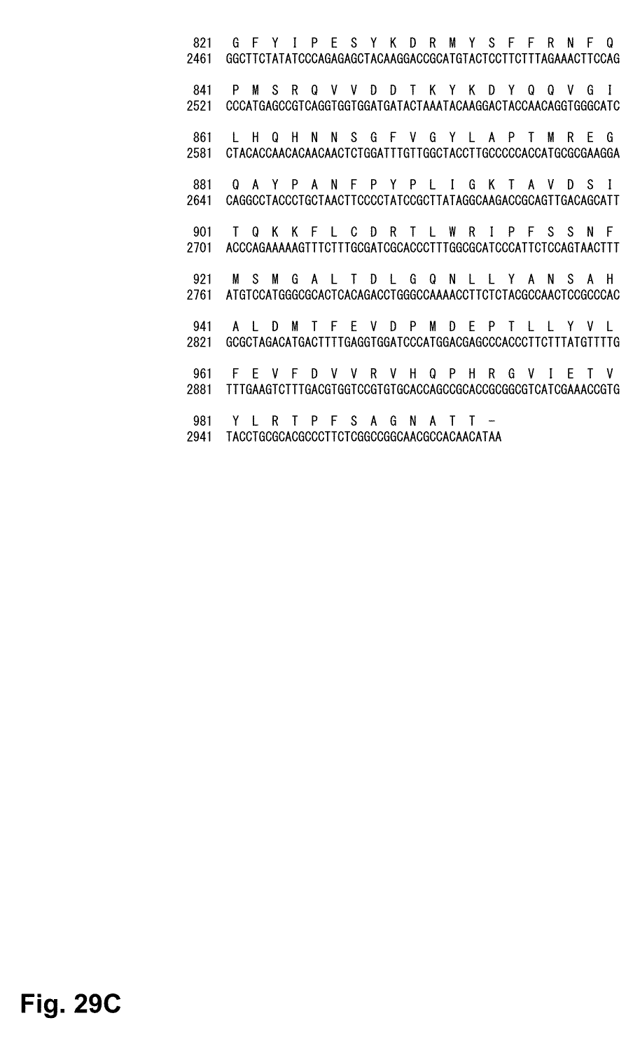

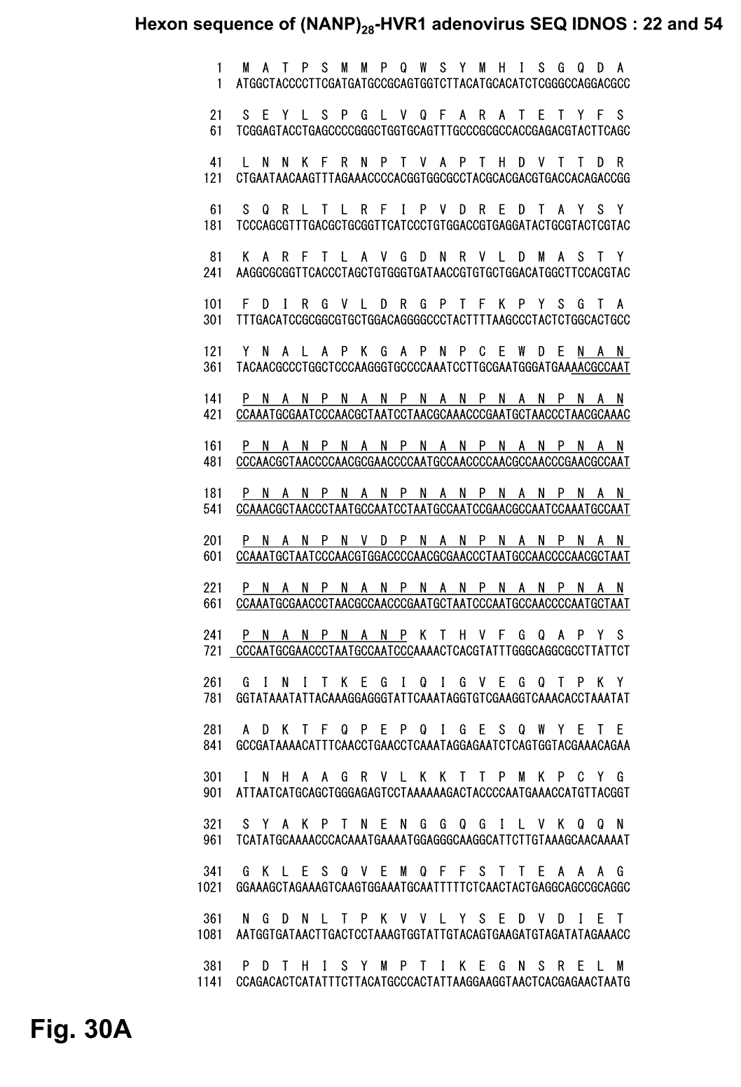

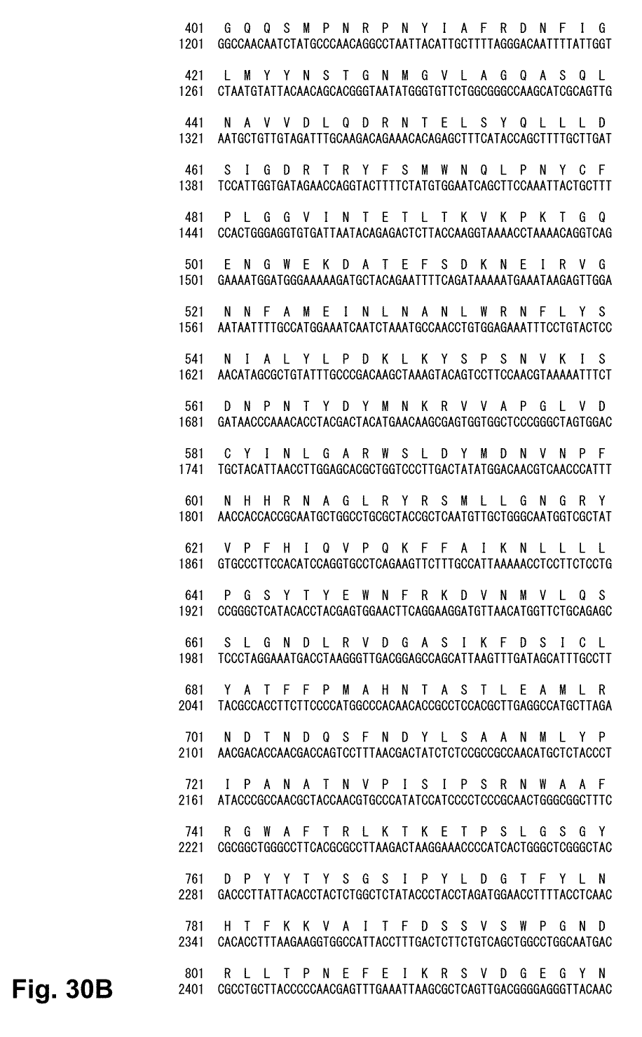

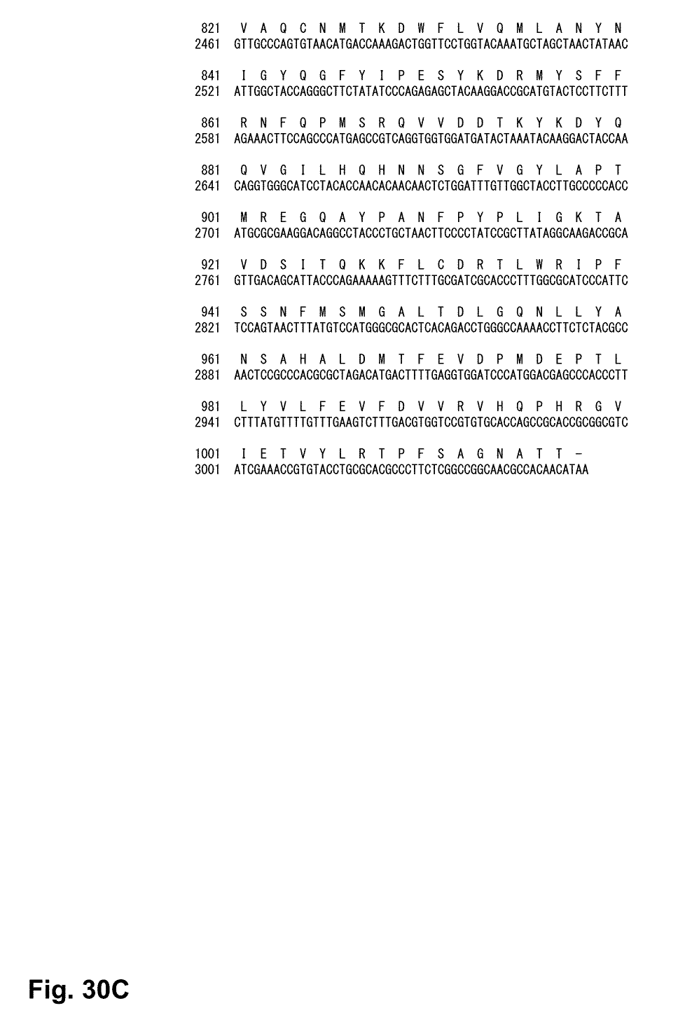

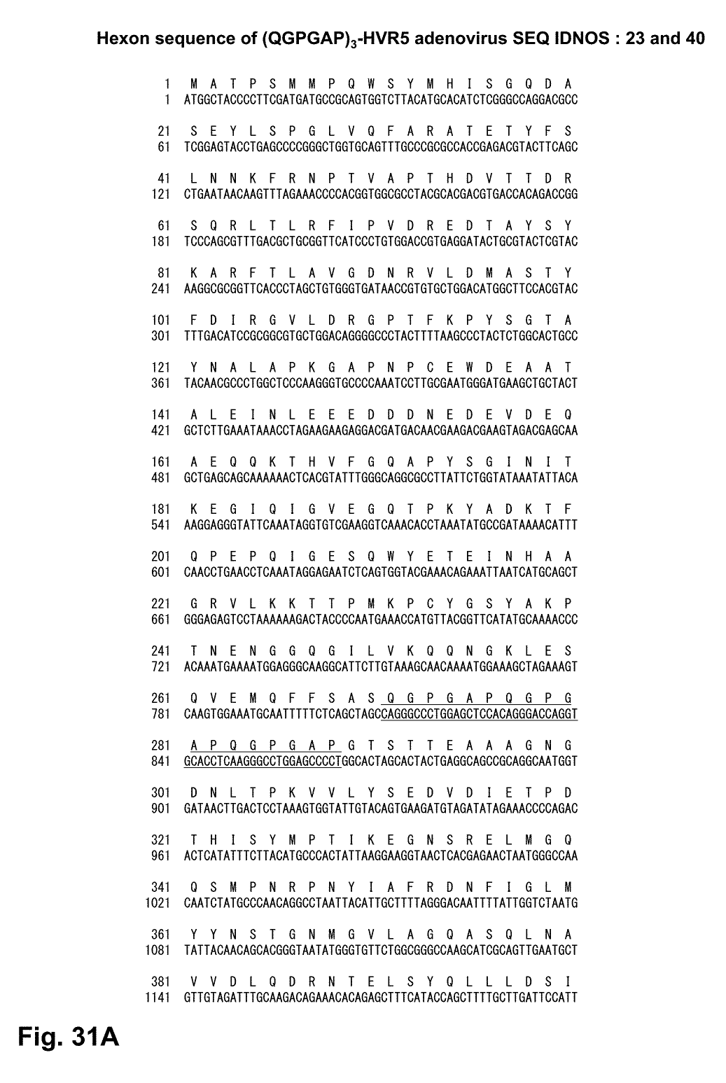

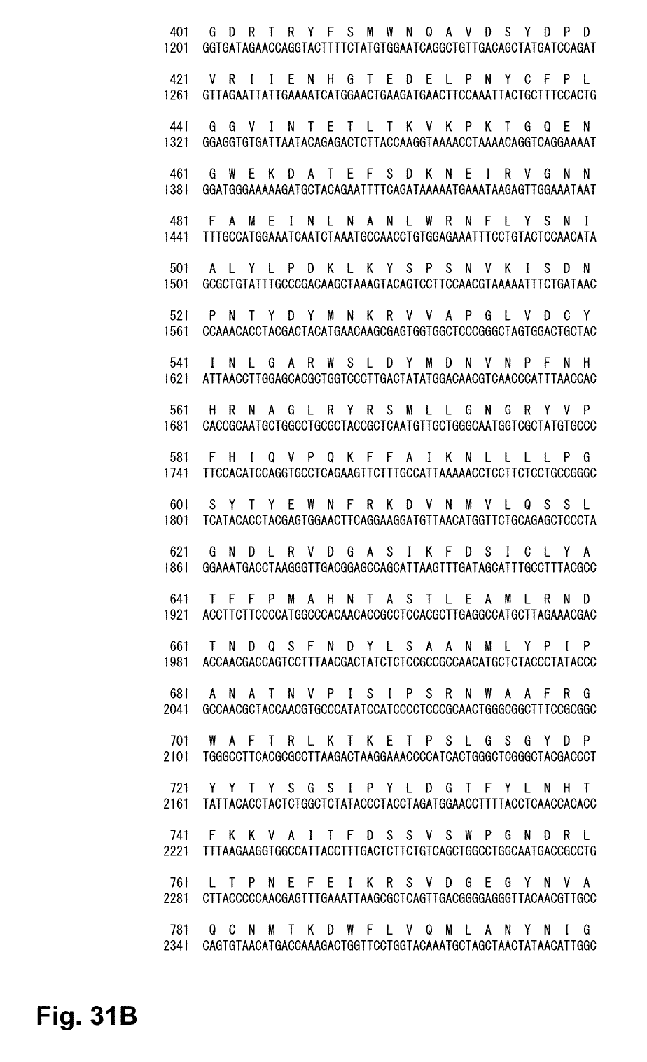

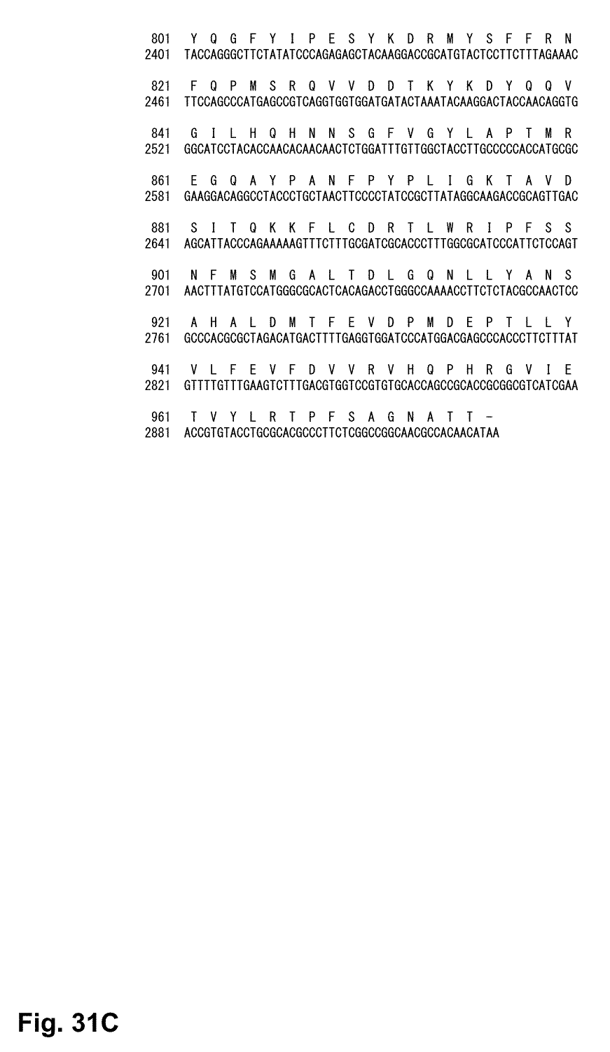

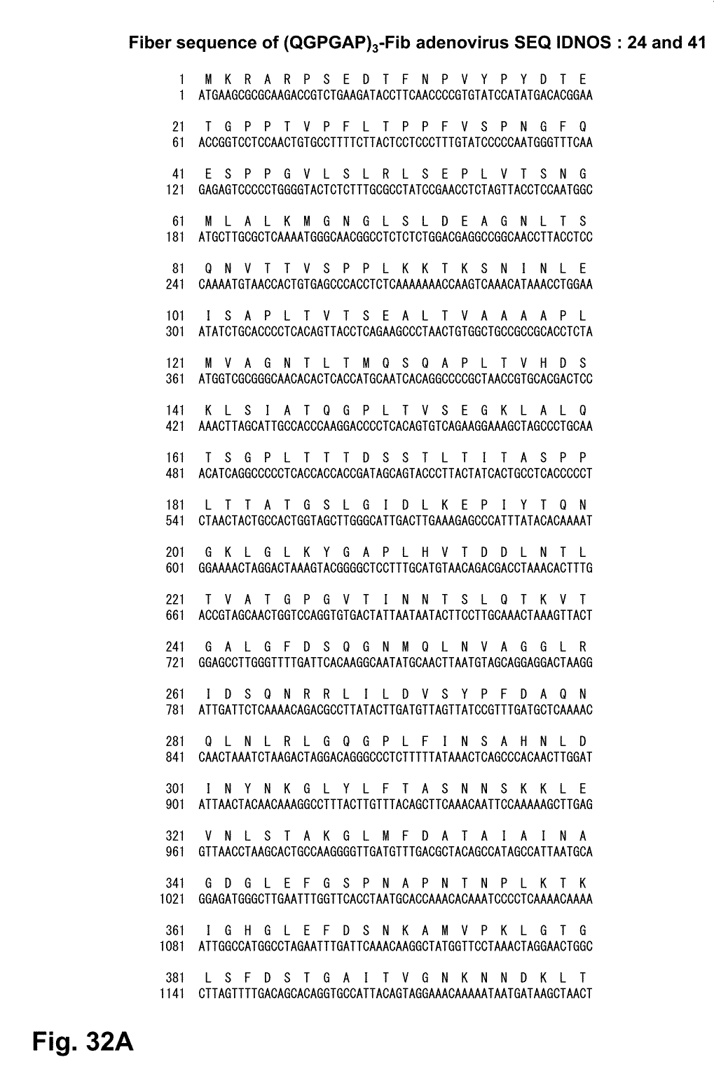

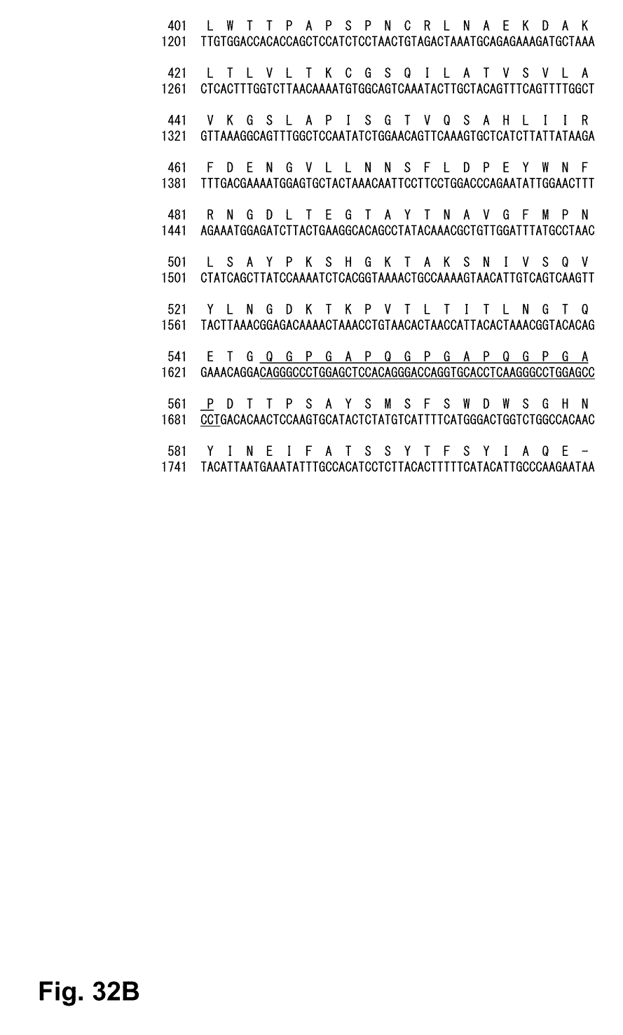

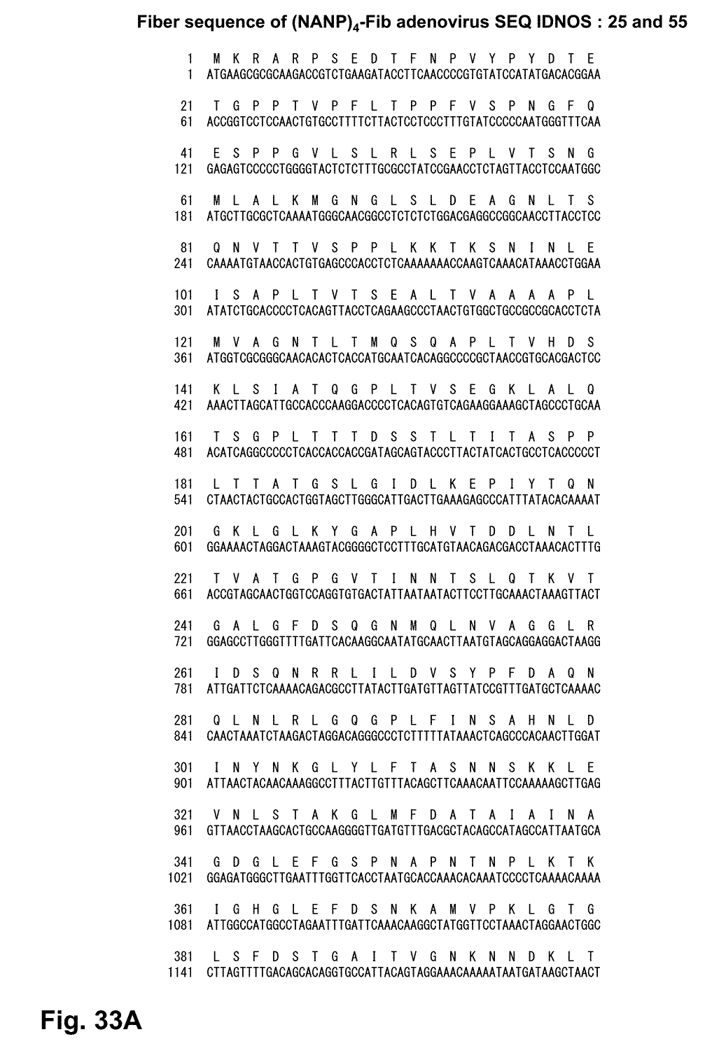

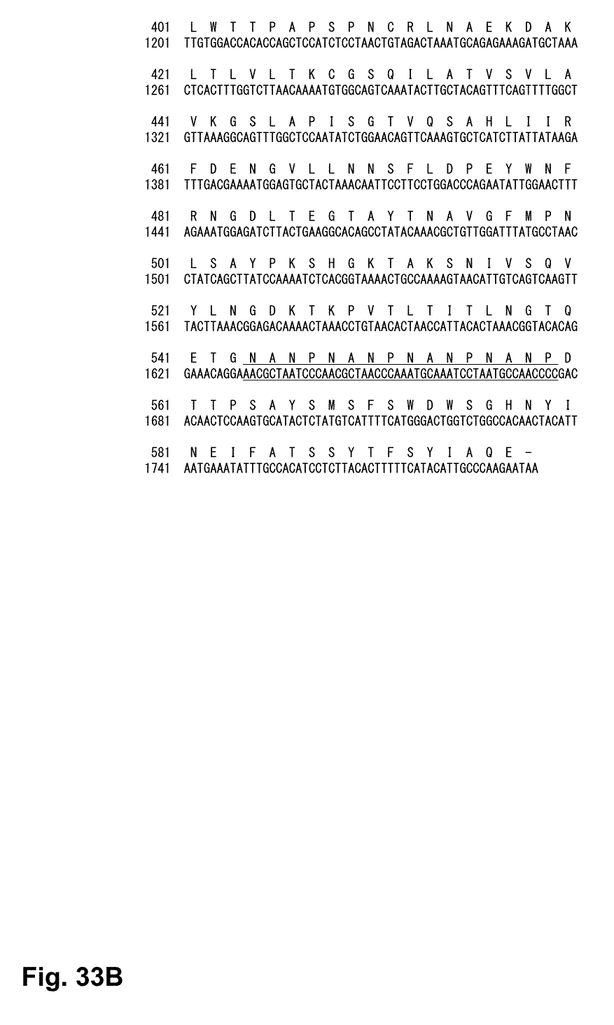

The one or more modified capsid and/or core proteins may be a modified Hexon protein, a modified Fiber protein, a modified pVII protein or a combination thereof. In one embodiment, a portion of a Hexon hypervariable region (HVR) and/or a portion of Fiber protein is replaced by at least one B-cell and/or T-cell epitope of a Plasmodium circumsporozoite protein. Alternatively, one or more B-cell and/or T cell epitope of a Plasmodium circumsporozoite protein may be inserted in the Fiber protein or Hexon HVR. In some aspects, the modified HVR may be HVR1, HVR2, HVR3, HVR4, HVR5, HVR6 or HVR7. In other aspects, the modified HVR may be HVR1 or HVR5. In some embodiments, the HVR-modified Hexon may have a nucleic acid sequence of SEQ ID NO:3 (FIG. 11), SEQ ID NO:4 (FIG. 12), SEQ ID NO:5 (FIG. 13), SEQ ID NO:6 (FIG. 14), SEQ ID NO:7 (FIG. 15), SEQ ID NO:8 (FIG. 16), SEQ ID NO:9 (FIG. 17), SEQ ID NO:10 (FIG. 18), SEQ ID NO:11 (FIG. 19), SEQ ID NO:12 (FIG. 20), SEQ ID NO:13 (FIG. 21), SEQ ID NO:14 (FIG. 22), SEQ ID NO:15 (FIG. 23), SEQ ID NO:16 (FIG. 24), SEQ ID NO:17 (FIG. 25), SEQ ID NO:18 (FIG. 26), SEQ ID NO:19 (FIG. 27), SEQ ID NO:20 (FIG. 28), SEQ ID NO:21 (FIG. 29), SEQ ID NO:22 (FIG. 30), or SEQ ID NO:23 (FIG. 31). In other embodiments, the modified Fiber protein may have a nucleic acid sequence of SEQ ID NO:24 (FIG. 32) or SEQ ID NO:25 (FIG. 33).

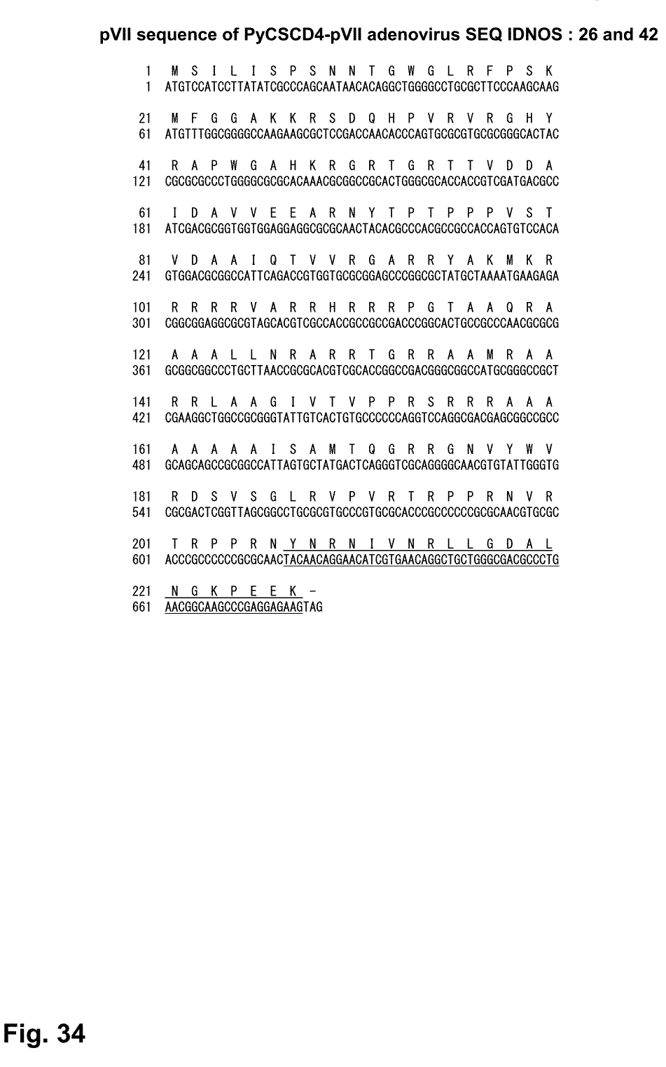

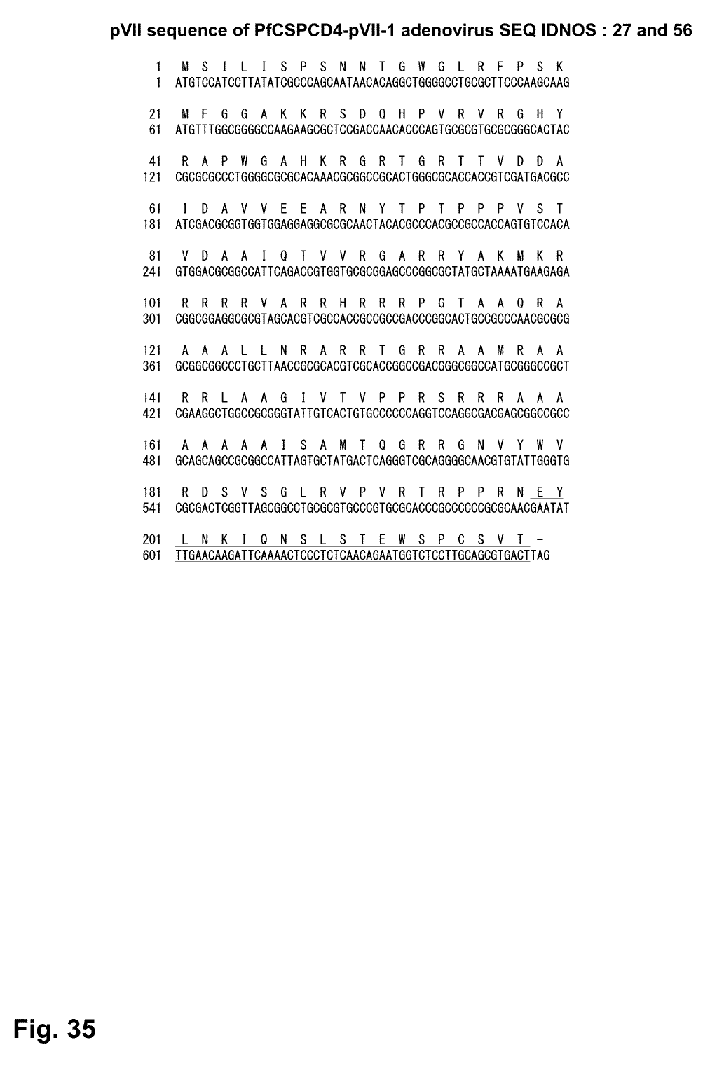

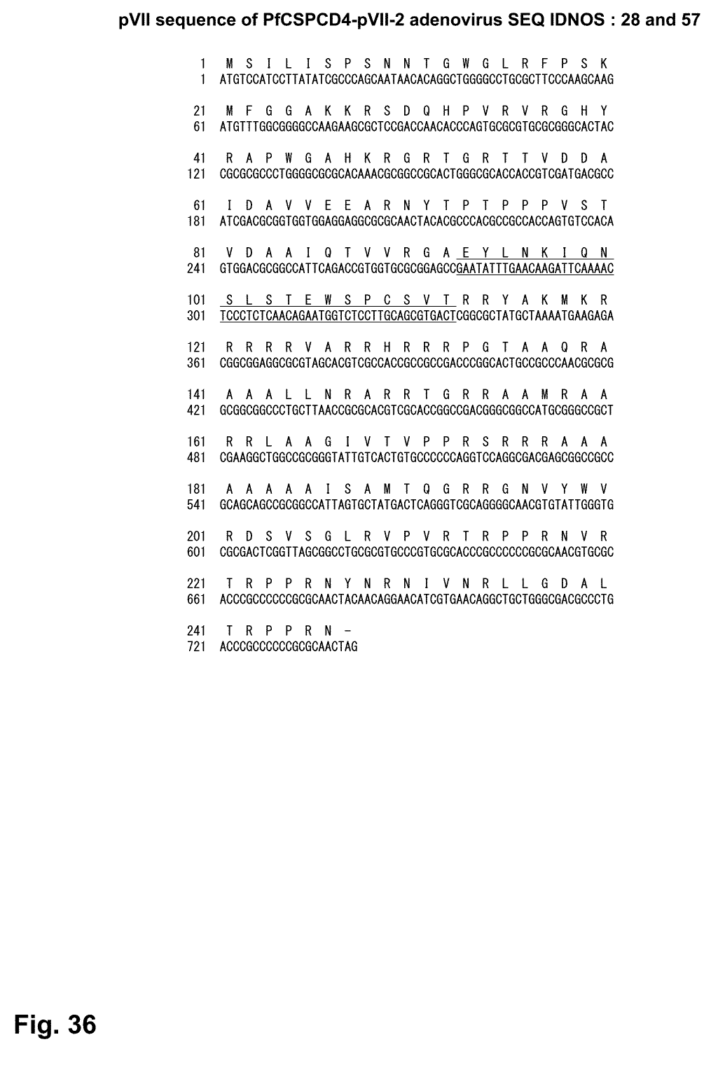

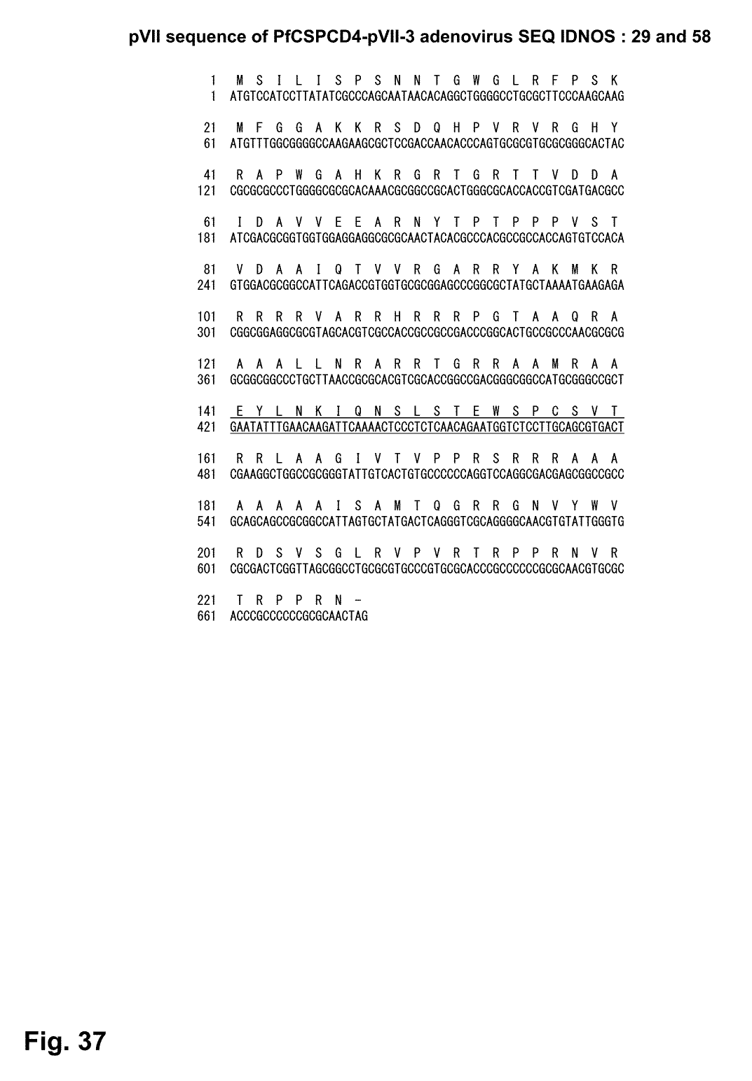

In another embodiment, a T-cell epitope of a Plasmodium circumsporozoite protein may be inserted into an adenovirus core pVII protein at any of the following sites: the C-terminus, before the first Nuclear Localization Signal (NLS) or between the two NLS. Alternatively, a T-cell epitope of a Plasmodium circumsporozoite protein may replace a portion of the pVII protein. In some embodiments, the modified pVII protein may have a nucleic acid sequence of SEQ ID NO:26 (FIG. 34), SEQ ID NO:27 (FIG. 35), SEQ ID NO:28 (FIG. 36) or SEQ ID NO:29 (FIG. 37).

In the recombinant adenovirus may express a transgenic protein or recombinant transgenic protein. In some embodiments, the transgenic protein or recombinant transgenic protein is a Plasmodium circumsporozoite protein or an antigenic determinant that is encoded by a recombinant adenovirus plasmid vector as described herein, and is expressed by a recombinant adenovirus produced by said recombinant adenovirus plasmid vector after infection of one or more host cells,

Thus, in some embodiments, the recombinant adenovirus plasmid vectors comprise a nucleotide sequence encoding a recombinant transgenic protein. In one embodiment, the recombinant transgenic protein may comprise an antigenic determinant of P. yoelii, a rodent-specific parasite, wherein the antigenic determinant comprises a P. yoelii circumsporozoite (CS) protein gene or an antigenic portion thereof. In another embodiment, the recombinant transgenic protein may comprise an antigenic determinant of P. falciparum, a human-specific parasite, wherein the antigenic determinant comprises a P. falciparum circumsporozoite gene (CS) protein or an antigenic portion thereof. The P. falciparum CS protein has demonstrated prevention of malaria when used as the basis of active immunization in humans against mosquito-borne infection. The antigenic determinant may further comprise an immunogenic epitope, such as a B cell and/or T cell epitope.

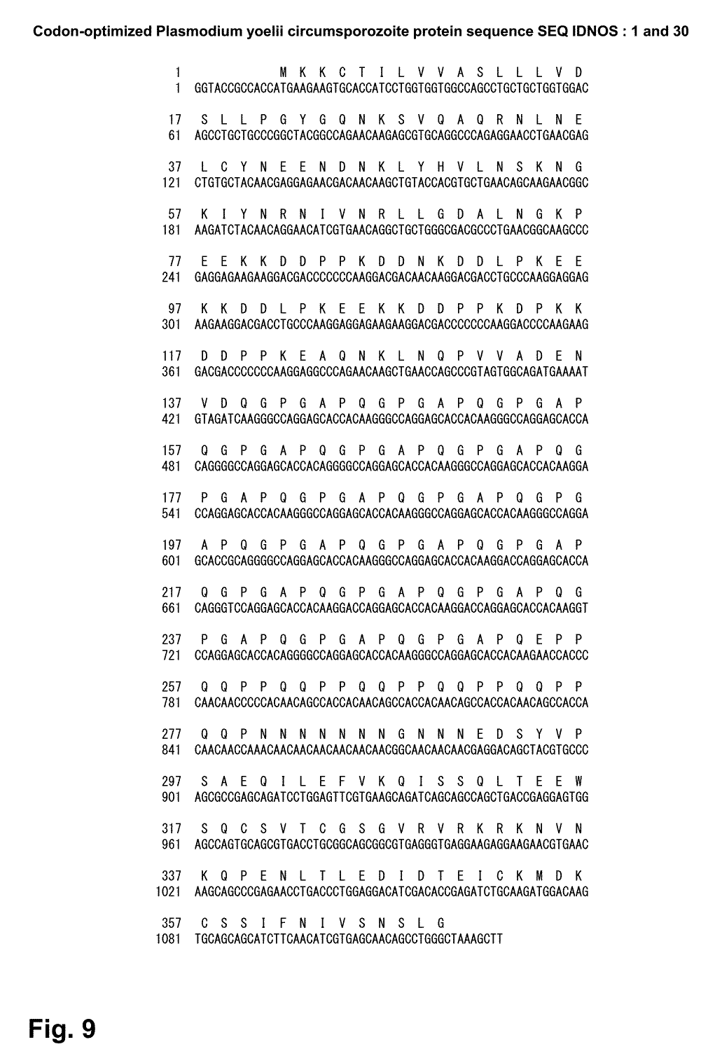

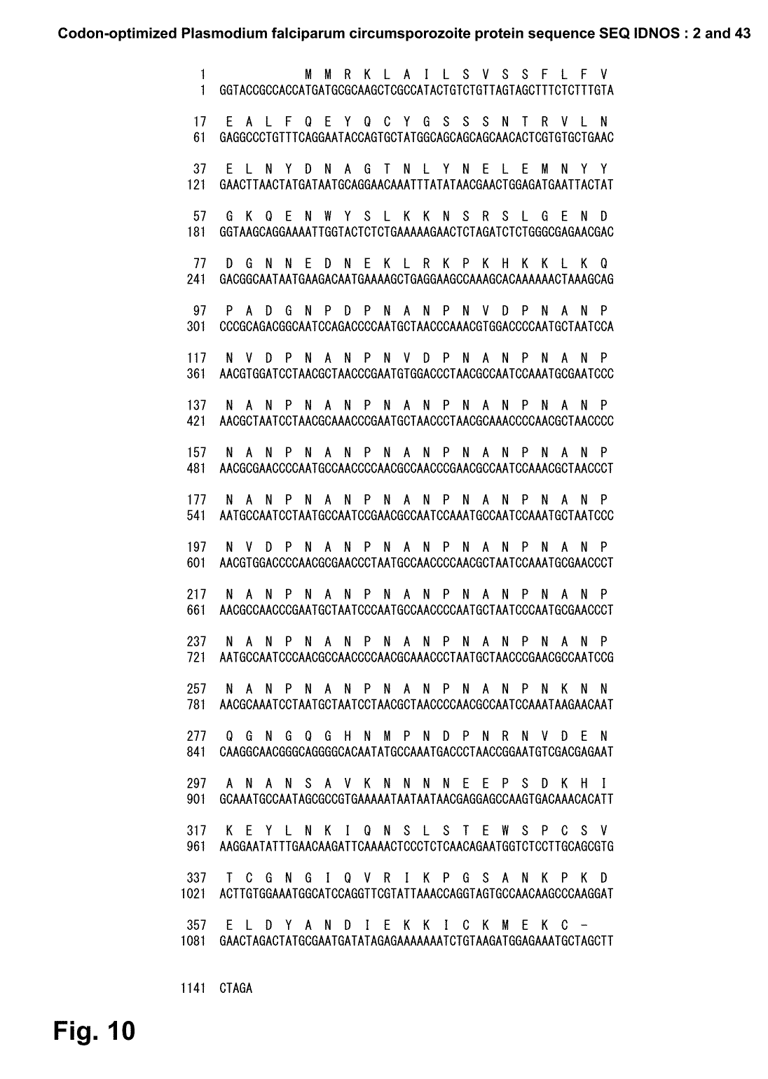

In some embodiments, the CS protein is codon-optimized for enhanced expression in a subject. Codon-optimization is based on the required amino acid content, the general optimal codon usage in the subject of interest as well as any aspects that should be avoided to ensure proper expression. Such aspects may be splice donor or acceptor sites, stop codons, polyadenylation (pA) signals, GC- and AT-rich sequences, internal TATA boxes, or any other aspects known in the art. In some embodiments, the DNA sequence of the codon-optimized CS transgene is shown in FIG. 9 (SEQ ID NO:1, P. yoelii) and FIG. 10 (SEQ ID NO: 2, P. falciparum).

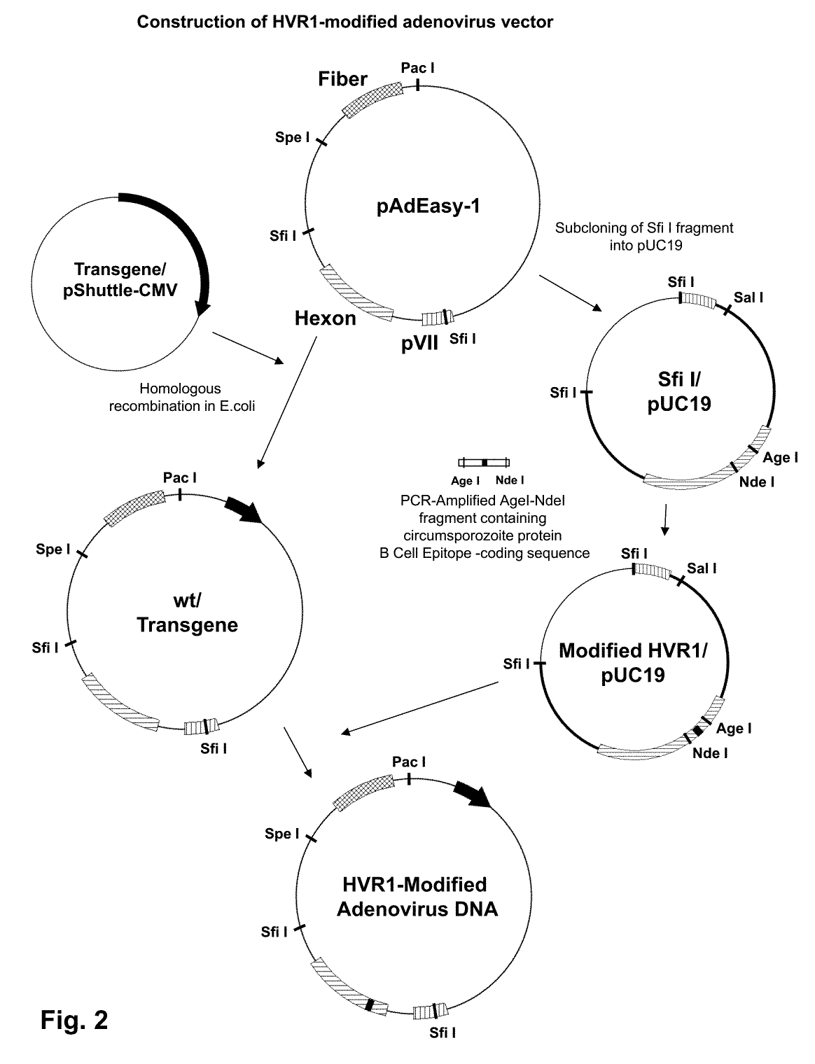

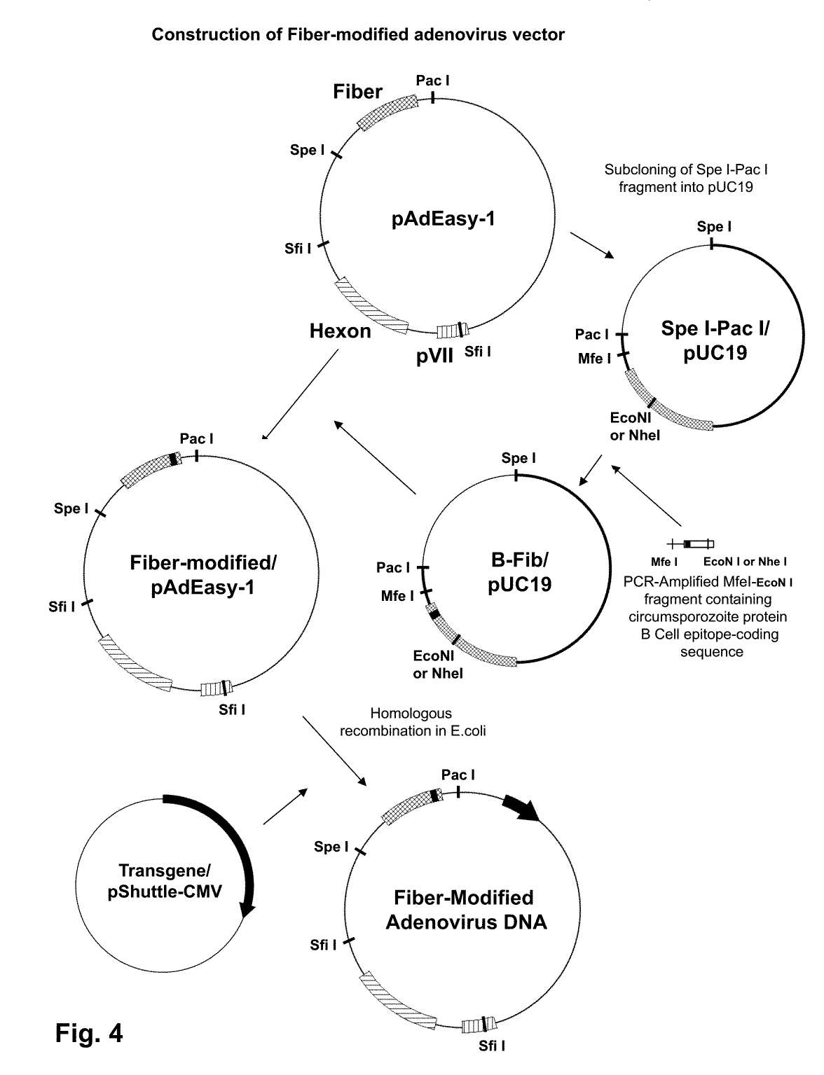

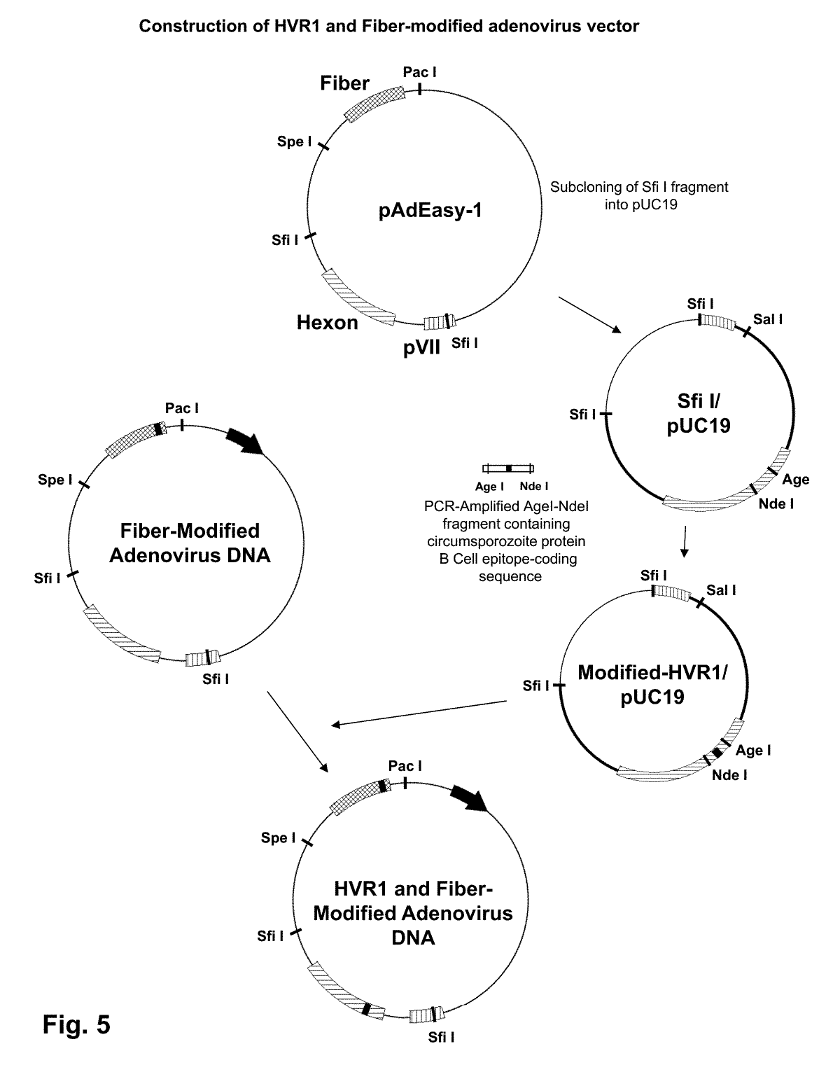

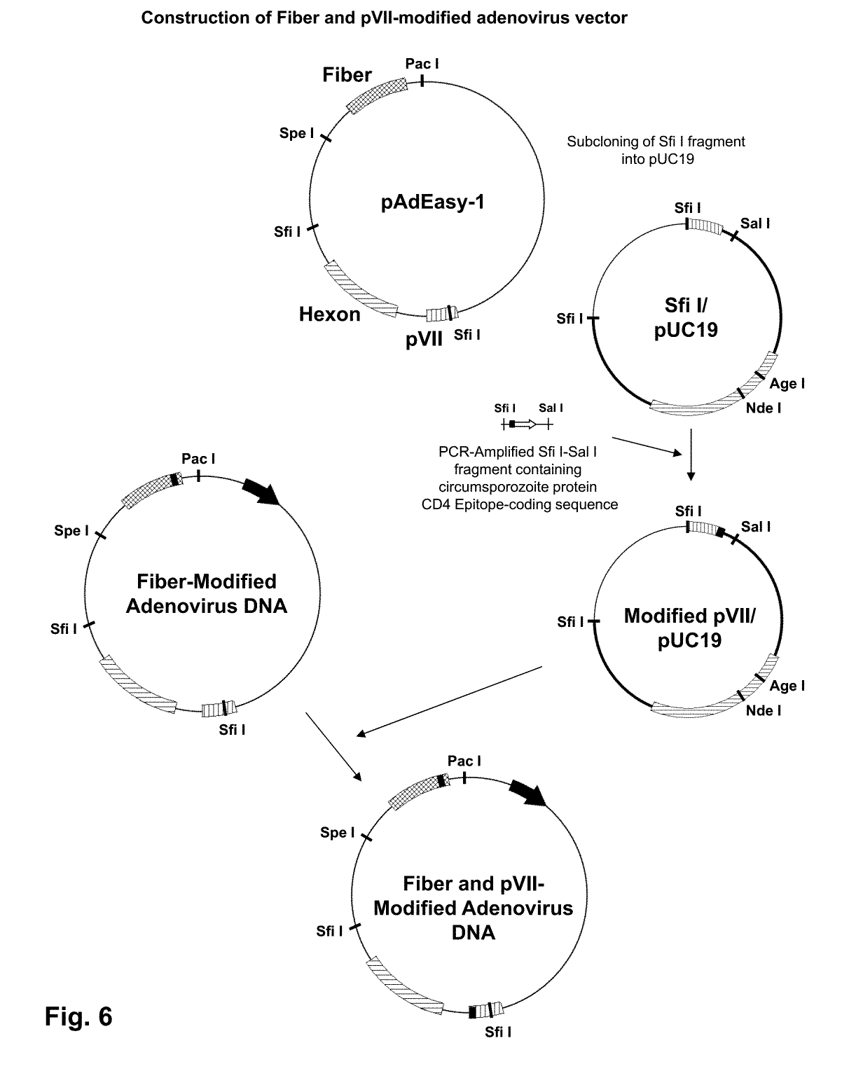

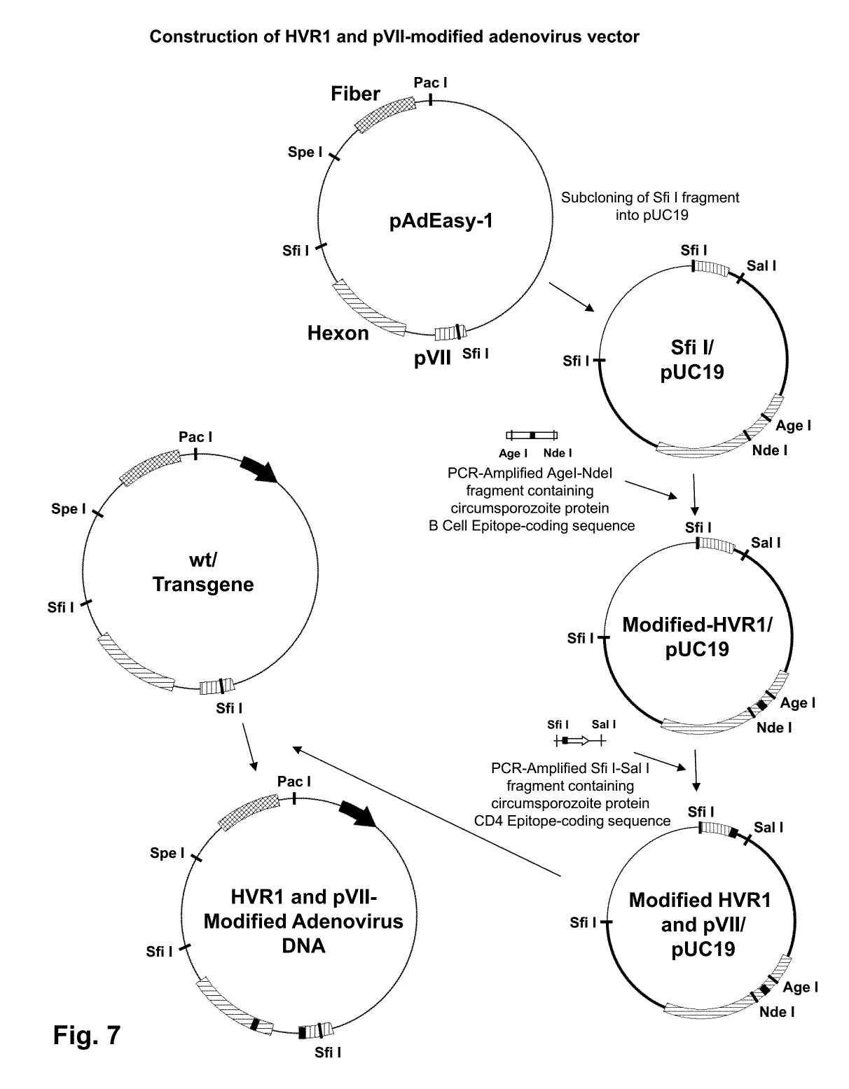

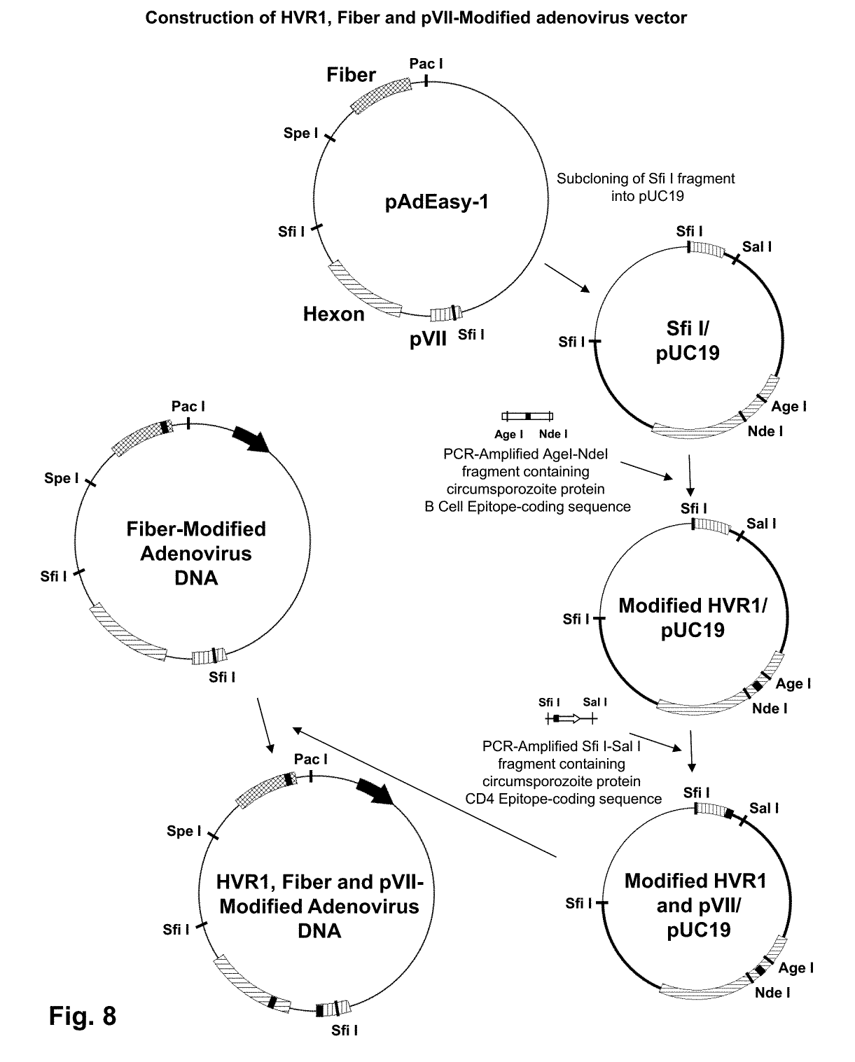

In some embodiments, the recombinant adenovirus plasmid vector may be one of the following modified P. falciparum recombinant adenovirus plasmid vectors: HVR1-modified adenovirus vector (NANP-HVR1/PfCSP) constructed as shown in FIG. 2, using a B cell epitope coding sequence of (NANP)ₙ (SEQ ID NO:60); Fiber-modified adenovirus vector (NANP-Fib/PfCSP) constructed as shown in FIG. 4, using a B cell epitope coding sequence of (NANP)ₙ (SEQ ID NO:60); HVR1 and Fiber-modified adenovirus vector (NANP-HVR1/B-Fib/PfCSP) constructed as shown in FIG. 5, using a B cell epitope coding sequence of (NANP)ₙ (SEQ ID NO:60); HVR1 and pVII-modified adenovirus vector (NANP-HVR1/CD4-pVII/PfCSP) constructed as shown in FIG. 7, using a B cell epitope of (NANP)ₙ (SEQ ID NO:60) and a CD4 epitope coding sequence of EYLNKIQNSLSTEWSPCSVT (SEQ ID NO:62); Fiber and pVII-modified adenovirus vector (NANP-Fib/CD4-pVII/PfCSP) constructed as shown in FIG. 6, using a B cell epitope of (NANP)ₙ (SEQ ID NO:60) and a CD4 epitope coding sequence of EYLNKIQNSLSTEWSPCSVT (SEQ ID NO:62); and HVR1, Fiber and pVII-modified adenovirus vector (NANP-HVR1/Fib/CD4-pVII/PfCSP) constructed as shown in FIG. 8, using a B cell epitope of (NANP)ₙ (SEQ ID NO:60) and a CD4 epitope coding sequence of EYLNKIQNSLSTEWSPCSVT (SEQ ID NO:62).

In other embodiments, the recombinant adenovirus plasmid vector may be one of the following modified P. yoelii recombinant adenovirus plasmid vectors: HVR1-modified adenovirus vector (QGPGAP-HVR1/PyCS) constructed as shown in FIG. 2, using a B cell epitope coding sequence of (QGPGAP)ₙ (SEQ ID NO:59); Fiber-modified adenovirus vector (QGPGAP-Fib/PyCS) constructed as shown in FIG. 4, using a B cell epitope coding sequence of (QGPGAP)ₙ (SEQ ID NO:59); HVR1 and Fiber-modified adenovirus vector (QGPGAP-HVR1/B-Fib/PyCS) constructed as shown in FIG. 5, using a B cell epitope coding sequence of (QGPGAP)ₙ (SEQ ID NO:59); HVR1 and pVII-modified adenovirus vector (QGPGAP-HVR1/CD4-pVII/PyCS) constructed as shown in FIG. 7, using a B cell epitope of (QGPGAP)ₙ (SEQ ID NO:59) and a CD4 epitope coding sequence of YNRNIVNRLLGDALNGKPEEK, (SEQ ID NO:61); Fiber and pVII-modified adenovirus vector (QGPGAP-Fib/CD4-pVII/PyCS) constructed as shown in FIG. 6, using a B cell epitope of (QGPGAP)ₙ (SEQ ID NO:59) and a CD4 epitope coding sequence of YNRNIVNRLLGDALNGKPEEK, (SEQ ID NO:61); and HVR1, Fiber and pVII-modified adenovirus vector (QGPGAP-HVR1/Fib/CD4-pVII/PyCS) constructed as shown in FIG. 8, using a B cell epitope of (QGPGAP)ₙ (SEQ ID NO:59) and a CD4 epitope coding sequence of YNRNIVNRLLGDALNGKPEEK, (SEQ ID NO:61).

In other embodiments, a recombinant adenovirus may be produced by one of the following modified P. falciparum or P. yoelii recombinant adenovirus plasmid vectors: NANP-HVR1/PfCSP or QGPGAP-HVR1/PyCS (FIG. 2), NANP-Fib/PfCSP or QGPGAP-Fib/PyCS (FIG. 4), NANP-HVR1/B-Fib/PfCSP or QGPGAP-HVR1/B-Fib/PyCS (FIG. 5), NANP-HVR1/CD4-pVII/PfCSP or QGPGAP-HVR1/CD4-pVII/PyCS (FIG. 7), NANP-Fib/CD4-pVII/PfCSP or QGPGAP-Fib/CD4-pVII/PyCS (FIG. 6), NANP-HVR1/Fib/CD4-pVII/PfCSP or QGPGAP-HVR1/Fib/CD4-pVII/PyCS (FIG. 8). The recombinant adenovirus may be produced in accordance with the methods described herein for producing a recombinant adenovirus plasmid vector with the ability to express a recombinant transgenic protein (e.g., Plasmodium CS protein) in mammalian host cells.

Purification of a recombinant adenovirus may be performed by using known virus purification methods. For example, purification of 0.5 to 1.0 mL of a stock virus obtained by the method of producing a recombinant adenovirus protein by inoculating insect cells (1×10⁷ cells/10 cm dish), such as AD293 cells. The culture supernatant is then collected several days after the infection, and a virus pellet obtained by centrifugation is suspended in a buffer, such as PBS (Phosphate Buffered Saline). The resulting suspension is subjected to a sucrose gradient of 10 to 60% and then centrifuged (25,000 rpm for 60 minutes at 4° C.) to collect a virus band. The collected virus is further suspended in PBS, subsequently centrifuged under the same conditions as above, and the resulting purified recombinant virus pellet is stored at 4° C. in a buffer, such as PBS.

Another embodiment is directed to a pharmaceutical composition essentially comprising at least one active ingredient. In one embodiment, an active ingredient of the pharmaceutical composition may comprise a recombinant adenovirus, which may be obtained by the genetic engineering techniques described herein. More specifically, the active ingredient may be a recombinant adenovirus comprising modified capsid and/or core proteins, wherein a portion of a Hexon hypervariable region (HVR), a portion of Fiber protein, a portion of pVII protein or a combination thereof is replaced by at least one immunogenic epitope of Plasmodium circumsporozoite protein. Alternatively, one or more B-cell and/or T cell epitope of a Plasmodium circumsporozoite protein may be inserted in the Fiber protein, Hexon HVR or pVII protein. The recombinant adenovirus plasmid vector further comprises a transgenic protein or recombinant transgenic protein that is expressed by the recombinant adenovirus after infecting one or more host cells. The transgenic protein or recombinant transgenic protein may be a Plasmodium circumsporozoite protein or a malaria antigen of a Plasmodium circumsporozoite protein, wherein the malaria antigen comprises at least one immunogenic epitope (e.g., a B cell or T cell epitope) of Plasmodium circumsporozoite protein.

In some embodiments, the active ingredient of the pharmaceutical composition is a recombinant adenovirus derived from a recombinant adenovirus plasmid vector, wherein the recombinant adenovirus plasmid vector is one of the following modified P. falciparum or P. yoelii recombinant adenovirus plasmid vectors: NANP-HVR1/PfCSP or QGPGAP-HVR1/PyCS (FIG. 2), NANP-Fib/PfCSP or QGPGAP-Fib/PyCS (FIG. 4), NANP-HVR1/B-Fib/PfCSP or QGPGAP-HVR1/B-Fib/PyCS (FIG. 5), NANP-HVR1/CD4-pVII/PfCSP or QGPGAP-HVR1/CD4-pVII/PyCS (FIG. 7), NANP-Fib/CD4-pVII/PfCSP or QGPGAP-Fib/CD4-pVII/PyCS (FIG. 6), NANP-HVR1/Fib/CD4-pVII/PfCSP or QGPGAP-HVR1/Fib/CD4-pVII/PyCS (FIG. 8). These recombinant adenovirus plasmid vectors are capable of producing recombinant adenoviruses when transfected into cells (e.g., AD293 cells) and wherein the recombinant transgenic protein may be expressed in mammalian cells, including human cells.

When given to a subject, a pharmaceutical composition having an active ingredient is a recombinant adenovirus as described herein enhances malaria infection-preventing effects against a malaria infectious antigen and reduces the infectivity titer, as described further in the Examples below. Thus, the recombinant adenovirus may be used for the treatment of malaria infections associated with infection of target cells and tissues. Examples of target cells affected by such malaria infection include blood cells, hepatic cells, renal cells, brain cells, lung cells, epithelial cells, and muscular cells. Examples of tissues comprising such cells include the lung, liver, kidney, brain, arteries and veins, the stomach, intestines, urethra, skin, and muscle.

In some aspects, the pharmaceutical composition may enhance malaria infection-preventing effects against infectious antigens, for example, malaria antigens such as sporozoite surface antigens (Circumsporozoite Protein (CSP) and Thrombospondin Related Adhesive Protein (TRAP)) of malaria parasites, merozoite surface membrane protein (MSPI), malaria S antigen secreted from erythrocytes infected with malaria, and P. falciparum Erythrocyte Membrane Protein-1 (PfEMPI) protein present in the knobs of erythrocytes infected with malaria. The pharmaceutical composition may enhance malaria infection-preventing effects against a Plasmodium parasite, selected from any known Plasmodium (P) species, for example, P. falciparum, P. malariae, P. ovale, P. vivax, P. knowlesi, P. berghei, P. chabaudi and P. yoelii, by reducing the infectivity titer. When administered to a subject, a reduction of the infectivity titer by the pharmaceutical composition may result in an increased survival, disease-free survival, or infection-free survival period and survival, disease-free survival, or infection-free survival rate when compared to subjects not administered the pharmaceutical composition. Thus, in some aspects, the pharmaceutical composition is useful as a preventive or therapeutic agent for malaria infections caused by pathogens such as Plasmodium. In further aspects, the pharmaceutical composition is useful as a preventive or therapeutic agent for complications resulting from a malaria infection caused by pathogens such as Plasmodium.

The infection-preventing effect of the recombinant adenovirus of the present invention in a subject can be provided, for example, by administering the pharmaceutical composition containing the capsid-modified recombinant adenovirus of the present invention and additives for pharmaceutical administration to vertebrates, particularly mammals, including humans, by intramuscular (i.m.), subcutaneous (s.c.), intracutaneous (i.c.), intradermal (i.d.), intraperitoneal (i.p.), nasal, or respiratory route, and then immunizing the vertebrates with the pharmaceutical composition containing the recombinant adenovirus described herein as an active ingredient several times. To evaluate the infection-preventing effect, the survival rate, disease-free survival, or infection-free survival of subjects immunized with the pharmaceutical composition several times followed by infection by a target pathogen (such as a selected Plasmodium species) may be compared with the survival rate, disease-free survival, or infection-free survival of subjects not given the pharmaceutical composition.

In some embodiments, the pharmaceutical composition may additionally comprise a pharmaceutically effective amount of capsid and/or core-modified recombinant adenovirus as described herein and a pharmaceutically acceptable carrier, which is described further below.

Another embodiment is directed to a vaccine composition essentially comprising at least one active ingredient. In one embodiment, an active ingredient of the vaccine composition may comprise a recombinant adenovirus, derived from a recombinant adenovirus plasmid vector as described herein. More specifically, the active ingredient may be a recombinant adenovirus comprising modified capsid or core proteins, wherein a portion of a Hexon hypervariable region (HVR), a portion of Fiber protein, a portion of pVII protein or a combination thereof are replaced by at least one immunogenic epitope of Plasmodium circumsporozoite protein. Alternatively, at least one immunogenic epitope of a Plasmodium circumsporozoite protein may be inserted in the pVII protein, Fiber protein or Hexon HVR, or a combination thereof. In some embodiments, the active ingredient of the vaccine composition may be derived from a recombinant adenovirus plasmid vector illustrated in FIGS. 2-8, for example, NANP-HVR1/PfCSP or QGPGAP-HVR1/PyCS (FIG. 2), NANP-Fib/PfCSP or QGPGAP-Fib/PyCS (FIG. 4), NANP-HVR1/B-Fib/PfCSP or QGPGAP-HVR1/B-Fib/PyCS (FIG. 5), NANP-HVR1/CD4-pVII/PfCSP or QGPGAP-HVR1/CD4-pVII/PyCS (FIG. 7), NANP-Fib/CD4-pVII/PfCSP or QGPGAP-Fib/CD4-pVII/PyCS (FIG. 6), NANP-HVR1/Fib/CD4-pVII/PfCSP or QGPGAP-HVR1/Fib/CD4-pVII/PyCS (FIG. 8).

In some aspects, the vaccine composition, when administered to a subject, first comprises a recombinant adenovirus having one or more antigenic portions of a Plasmodium CS protein (i.e., a B cell epitope, T cell epitope or both) inserted into or replacing at least a part of a capsid or core protein. The vaccine composition may then express a recombinant transgenic protein, wherein the recombinant transgenic protein is a Plasmodium CS protein comprising a B cell epitope, T cell epitope or both. The antigenic portions of the Plasmodium CS protein are found in the recombinant transgenic protein and the modified capsid or core proteins promote or enhance acquired humoral immunity, cellular immunity, or both as described in the Examples below. Thus, in some aspects, the recombinant adenovirus as described herein is useful as a vaccine to promote or enhance humoral immunity, cellular immunity, or both.

In further embodiments, the vaccine composition may enhance infection-preventing effects against infectious antigens, for example, malaria antigens such as sporozoite surface antigens (CSP and TRAP) of malaria parasites, merozoite surface membrane protein MSPI, malaria S antigen secreted from erythrocytes infected with malaria, PfEMPI protein present in the knobs of erythrocytes infected with malaria, Serine-Rich Antigen (SERA) protein, Tyrosine-Rich Acidic Matrix Protein (TRAMP), and Apical Membrane Antigen-1 (AMAI) protein. Further, a reduced infectivity titer (e.g., the viral infectivity titer) resulting from administration of a vaccine composition described herein may result in an increased survival, disease-free survival or infection-free survival period and survival, disease-free survival or infection-free survival rate when compared to subjects not administered the vaccine composition. Thus, in some aspects, the vaccine composition is also useful as a preventive or therapeutic agent for malaria infections caused by pathogens such as Plasmodium. In further aspects, the vaccine composition is also useful as a preventive or therapeutic agent for complications resulting from a malaria infection by pathogens such as Plasmodium.

A vaccine composition as described herein may comprise a therapeutically effective amount of a recombinant adenovirus as described herein, and further comprising a pharmaceutically acceptable carrier according to a standard method. Examples of acceptable carriers include physiologically acceptable solutions, such as sterile saline and sterile buffered saline.

In some embodiments, the vaccine or pharmaceutical composition may be used in combination with a pharmaceutically effective amount of an adjuvant to enhance the anti-malaria effects. Any immunologic adjuvant that may stimulate the immune system and increase the response to a vaccine, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphoryl-lipid A/trehalose dicorynoycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, N-acetylmuramine-L-alanyl-D-isoglutamine (MDP), thymosin al and MF59. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In some embodiments, the vaccine or pharmaceutical composition described herein may be used in combination with other known pharmaceutical products, such as immune response-promoting peptides and antibacterial agents (synthetic antibacterial agents). The vaccine or pharmaceutical composition may further comprise other drugs and additives. Examples of drugs or additives that may be used in conjunction with a vaccine or pharmaceutical composition described herein include drugs that aid intracellular uptake of the recombinant adenovirus or recombinant transgenic protein of the present invention, liposome and other drugs and/or additives that facilitate transfection, (e.g., fluorocarbon emulsifiers, cochleates, tubules, golden particles, biodegradable microspheres, and cationic polymers).

In some embodiments, the amount of the active ingredient contained in the vaccine or pharmaceutical composition described herein may be selected from a wide range of concentrations, Virus Particle Unit (VPU), Plaque Forming Unit (PFU), weight to volume percent (w/v %) or other quantitative measure of active ingredient amount, as long as it is a therapeutically or pharmaceutically effective amount. The dosage of the vaccine or pharmaceutical composition may be appropriately selected from a wide range according to the desired therapeutic effect, the administration method (administration route), the therapeutic period, the patient's age, gender, and other conditions, etc.

In some aspects, when a recombinant adenovirus is administered to a human subject as an active ingredient of the vaccine or pharmaceutical composition, the dosage of the recombinant adenovirus may be administered in an amount approximately corresponding to 10² to 10¹⁴ PFU, preferably 10⁵ to 10¹² PFU, and more preferably 10⁶ to 10¹⁰ PFU per patient, calculated as the PFU of the recombinant virus.

In further aspects, when a recombinant adenovirus is administered to a subject as an active ingredient of the vaccine or pharmaceutical composition, the dosage may be selected from a wide range in terms of the amount of expressible DNA introduced into the vaccine host or the amount of transcribed RNA. The dosage also depends on the strength of the transcription and translation promoters used in any transfer vectors used.

In some embodiments, the vaccine composition or pharmaceutical composition described herein may be administered by directly injecting a recombinant adenovirus suspension prepared by suspending the recombinant adenovirus in PBS (phosphate buffered saline) or saline into a local site (e.g., into the lung tissue, liver, muscle or brain), by nasal or respiratory inhalation, or by intravascular (i.v.) (e.g., intra-arterial, intravenous, and portal venous), subcutaneous (s.c.), intracutaneous (i.c.), intradermal (i.d.), or intraperitoneal (i.p.) administration. The vaccine or pharmaceutical composition of the present invention may be administered more than once. More specifically, after the initial administration, one or more additional vaccinations may be given as a booster. One or more booster administrations can enhance the desired effect. After the administration of the vaccine or pharmaceutical composition, booster immunization with a pharmaceutical composition containing the recombinant adenovirus as described herein may be performed.

In further embodiments, use of various other adjuvants, drugs or additives with the vaccine of the invention, as discussed above, may enhance the therapeutic effect achieved by the administration of the vaccine or pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); and PEG.

The vaccine or pharmaceutical composition containing a recombinant adenovirus described herein may be stored as an aqueous solution or a lyophilized product in a unit or multiple dose container such as a sealed ampoule or a vial.

Another embodiment further provides a method of preventing malaria infection, or a method of treating malaria comprising administering an effective amount of the recombinant adenoviral vaccine, formulation, or pharmaceutical composition. The present invention further provides a method of immunostimulation comprising administering an effective amount of a recombinant adenoviral vaccine composition, formulation, pharmaceutical composition or a combination thereof to a subject. Subjects may include humans, animals (such as mammals, birds, reptiles, fish, and amphibians), or any other subjects that may become infected with a malaria parasite. Malaria parasites may include a Plasmodium parasite, selected from any of known Plasmodium (P) species, for example, P. falciparum, P. malariae, P. ovale, P. vivax, P. knowlesi, P. berghei, P. chabaudi and P. yoelii.

In some embodiments, a recombinant adenovirus as described herein may be formed alone or may be together with a pharmaceutically acceptable carrier into a vaccine composition, formulation, or pharmaceutical composition, and administered to the subject. The administration route may be, for example, any administration route mentioned above. The pharmaceutically acceptable carrier for use in the present invention can be suitably selected from carriers commonly used in this technical field, according to the form of the pharmaceutical composition to be produced. For example, when the pharmacological composition is formed into an aqueous solution, purified water (sterile water) or a physiological buffer solution can be used as the carrier. When the pharmaceutical composition is formed into other appropriate solutions, organic esters capable of being injected, such as glycol, glycerol and olive oil may be used as the carrier. The composition may contain stabilizers, excipients and other commonly used substances in this technical field, and particularly in the field of vaccine formulations.

In further embodiments, the amount of recombinant adenovirus used in a vaccine composition, formulation, or pharmaceutical composition may be suitably selected from a wide range of concentrations, VPU, PFU, weight to volume percent (w/v %) or other quantitative measure of active ingredient amount. In some aspects, a suitable range of recombinant adenovirus in the composition is preferably about 0.0002 to about 0.2 (w/v %), and more preferably 0.001 to 0.1 (w/v %). The method of administration of a recombinant adenovirus vaccine composition, formulation, or pharmaceutical composition according to some embodiments may be suitably selected according to the dosage form, the patient's age, gender and other conditions such as the severity of the disease. A suitable dosage form is a form for parenteral administration, such as injections, drops, nasal drops, and inhalants. When the composition is formed into an injection or drops, the injection can be intravenously administered and mixed with a replacement fluid such as a glucose solution or an amino acid solution as appropriate, or can be administered intramuscularly (i.m.), intracutaneously (i.c.), subcutaneously (s.c.) intradermally (i.d.), or intraperitoneally (i.p.).

In other embodiments, the daily dosage of a recombinant adenovirus vaccine composition, formulation, or pharmaceutical composition may vary depending on the subject's condition, body weight, age, gender, etc. In some aspects, the dosage of a recombinant adenovirus is administered in an amount of approximately 0.001 to 100 mg per kg of body weight per day. The vaccine, formulation, or composition of the invention may be administered in one or more administrations per day.

In further embodiments, when a recombinant adenovirus is administered to a human subject as an active ingredient of the vaccine composition, formulation or pharmaceutical composition, the dosage of the recombinant adenovirus is administered in an amount approximately corresponding to 10² to 10¹⁴ PFU, preferably 10⁵ to 10¹² PFU, and more preferably 10⁶ to 10¹⁰ PFU per patient, calculated as the PFU of the recombinant adenovirus particle. The vaccine composition of the present invention should be administered according to Good Medical Practice, considering the clinical condition (for example, the condition to be prevented or treated) of each patient, the delivery site of the vaccine composition containing the recombinant adenovirus, the target tissue, the administration method, the dosage regimen, and other factors known to those skilled in the art. Therefore, the proper dosage of the vaccine composition herein is determined in consideration of the above.

Yet another embodiment of the disclosure relates to a method of treating or preventing a malaria infection in a subject, the method comprising administering an immunologic or therapeutic amount of a malaria vaccine composition comprising a recombinant adenovirus. The recombinant adenovirus of the malaria vaccine may comprise an antigenic determinant of a Plasmodium parasite, and may further comprise one or more modified capsid or core proteins. An immunologic, pharmacologic or therapeutic amount may be any suitable amount wherein a potent immune response is generated against one or more antigenic portions of the (CS) protein (i.e., the transgene, B cell epitope, or CD4+ T cell epitope) such that malarial infection is prevented or reduced in severity.

When a subject is first exposed or “primed” to an adenovirus vector, the immune system produces neutralizing antibodies against that specific vector. The immune response to the adenovirus is generally directed against the capsid proteins. Therefore, subsequent exposure to the same adenovirus vector, or “boosts,” can reduce the efficacy of transgene expression. Therefore, in some embodiments, the method of treating or preventing a malaria infection described above may comprise a priming step using a first recombinant adenovirus vector followed by one or more boosting steps using one or more different recombinant adenovirus vectors. This method may be used in subjects that have not yet been exposed to a wild-type adenovirus, or in a subject that has been previously exposed to a wild-type adenovirus vector, wherein the priming step recombinant adenovirus vector is used to circumvent existing adenovirus immunity. Further embodiments and examples are described below.

Adenovirus as a Vector

Adenoviruses are non-enveloped DNA viruses comprising a set of viral capsid proteins (described below) and a viral genome, that have been widely used to deliver one or more therapeutic or antigenic transgene to a variety of cells in vitro and in vivo. Many adenovirus serotypes exist. Of the known adenovirus serotypes, serotype 5 (Ad5) is preferably used as a vector for foreign gene transduction because of its strong infectivity in vivo (Abbink et al. 2007). Expression of the antigenic transgene may be controlled by any promoter or enhancer element known in the art. Promoters which may be used to control gene expression include, but are not limited to, cytomegalovirus immediate early promoter (CMV), simian virus 40 (SV40) early promoter, cellular polypeptide chain elongation factor 1 alpha (EF1) promoter, Rous sarcoma virus (RSV) promoter, and tetracycline-regulated (TR) promoter. A polyadenylation (pA) signal after the coding sequence may also be used for efficient transcription and translation. The recombinant adenovirus vector described herein may be replication-defective, having a deletion at least in the E1 region of the adenoviral genome, since the E1 region is required for replication, transcription, translation and packaging processes. In some aspects, the E2, E3 and/or E4 regions may also be deleted. In further aspects, a Kozak consensus sequence may be used for a more efficient translation (Kozak 1987).

The adenovirus (Ad) system is an attractive vector for the development of recombinant vaccines for a number of reasons. One reason is that recombinant adenoviral vectors infect most mammalian cell types (both replicative and non-replicative), including, but not limited to, mouse and human cell types. Thus, the same vector may be used successfully in mouse models and human clinical trials alike. Another reason is that any transferred genetic information remains epichromosomal, avoiding insertional mutagenesis and alteration of the cellular genotype (Crystal 1995). Yet another reason is that the transgene remains unaltered after successive rounds of viral replication. Other advantages of using adenovirus include that recombinant adenovirus: 1) has a high virion stability, 2) is well tolerated, 3) may be grown at high titer, 4) can accommodate large transgenes, 5) has a genome that has been extensively studied for many years such that the complete DNA sequence of several serotypes is known, facilitating the manipulation of the Ad genome by recombinant DNA techniques (Graham and Prevec 1992).

In one embodiment, the adenovirus vaccine platform is used as a viral vector for development of a vaccine that targets a pre-erythrocytic malaria parasite, and provides protection from malaria infection. Among known recombinant viral vectors (Rodrigues et al. 1997, Bruña-Romero et al. 2001, Anderson et al. 2004, Tao et al. 2005), adenovirus has been shown to be a suitable viral vector for a malaria vaccine because it can induce a strong protective cellular immune response to pre-erythrocytic malaria parasites (Rodrigues et al. 1997). The malaria parasite may be any one of the Plasmodium family. In some embodiments, the targeted parasite may be P. yoelii or P. falciparum.

Adenovirus Vectors Expressing PyCS as a Transgene Elicits a Malaria-Specific CD8+ T Cell Response

Adenovirus is an attractive vector for inducing a significant CD8+ T cell-mediated protective immunity against malaria (Rodrigues et al. 1997, Rodrigues et al. 1998). The immunogenicity of a recombinant adenovirus expressing the P. yoelii (a rodent malaria parasite) CS protein, AdPyCS, was determined using a rodent malaria model. The inoculation of mice with AdPyCS induces complete immunity in a significant proportion of mice, preventing the occurrence of parasitemia (Rodrigues et al. 1997). This protective effect is primarily mediated by CD8+ T cells, as evidenced by depletion of the T cell population and is corroborated by the fact that AdPyCS was unable to induce high titers of antibody response against malaria parasites.

To quantitatively measure the infectivity of capsid-modified adenovirus, the shuttle vector may contain a GFP expression cassette and cloning sites for a transgene. The resulting shuttle vector (GFP/pShuttle-CMV) has dual pCMV promoters and SV40pAs for a transgene and GFP from pmaxGFP (Amaxa, Germany). The optimized PyCS fragment was inserted into KpnI and HindIII sites of GFP/pShuttle-CMV.

The immunogenicity of Ad(PyCS+GFP) was determined by measuring the magnitude of the CS-specific CD8+ T cell response and the level of protective immunity against the plasmodial liver stages. Administration of Ad(PyCS+GFP) via different routes, at an optimal dose, 10¹⁰ viral particle (v.p.) elicited the same pattern of anti-malarial protective responses that AdPyCS was shown to elicit, with the s.c. and i.m. routes inducing the strongest response resulted in the highest degree of liver stage inhibition in mice challenged with live P. yoelii sporozoites. This illustrates that as a vaccine, Ad(PyCS+GFP) behaves equivalently to AdPyCS (Rodrigues et al 1997), and is a potentially useful tool in determining the in vivo tropism of AdPyCS.

Adenovirus Capsid and Core Proteins

The studies above confirm that recombinant adenoviral vectors expressing a CS protein elicit a strong cellular immune response by CD8+ T cells, but no appreciable humoral response. Therefore, because the humoral response to wild-type adenovirus can often be attributed to capsid proteins, recombinant adenoviral vectors with modified capsid and core proteins were constructed to 1) enhance humoral immunity via B cell activation, 2) enhance humoral immunity via T helper cell activation, and 3) circumvent existing adenoviral immunity.

Adenovirus is a non-enveloped naked double stranded DNA virus with an icosahedral shape, having 20 faces of equilateral triangles. The adenovirus capsid consists of 252 capsomers, of which 240 are Hexon trimers and 12 are penton pentamers. A Fiber protein, which projects from each penton base, mediates attachment to host cells by interaction with the cellular receptor. A secondary interaction occurs between the RGD (Asp-Arg-Gly) motif in the penton base with avβ3, avβ5 and similar integrins, facilitating subsequent internalization of adenovirus into the cell (Mathias et al. 1994, Wickham et al. 1993). Most of the adenovirus use the coxsackie-adenovirus receptor, CAR, as a cellular receptor (Bergelson et al. 1997). In addition, MHC class I molecules, VCAM, and heparan sulfate, are shown to mediate attachment and entry of Ad5 (Chu et al. 2001, Hong et al. 1997). Following entry via endocytosis, the Ad5 rapidly escapes from endocytic compartments into the cytosol (Meier and Greber 2003, Leopold and Crystal 2007). The virion then translocates to the nucleus using microtubules. The Fiber protein is shed as the earliest capsid protein in the process (Nakano et al. 2000, Hong et al 2003). Adenoviruses of different serotypes demonstrate different trafficking patterns (Miyazawa et al. 1999, Miyazawa et al. 2001). Changing or modifying the Fiber protein can impact trafficking, which may be particularly important with regard to antigen processing and presentation, following infection of antigen presenting cells (APC).

The adenovirus Fiber is a trimer divided into Fiber tail, shaft and knob domains (Henry et al. 1994, Rux and Burnett 2004, Chroboczek et al. 1995). The three dimensional structure of the knob domain is known, and together with mutagenesis studies, these studies allow the areas involved in CAR interaction and trimerization to be visualized (Kirby et al. 1999, Xia et al. 1995). The Fiber shaft projects from the virion and the Fiber knob contains the Coxsackie and Adenovirus Receptor (CAR) interaction domain (Roelvink et al. 1999, Bewley et al. 1999). The CAR-binding site of the Fiber knob consists primarily of residues from the AB loop and CD loop and extends secondarily to the FG and HI loop and the B, E and F β sheets (Roelvink et al. 1999, Bewley et al. 1999). The HI loop has been the best studied insertion site on the Fiber knob (Worgall et al. 2004, Mizuguchi and Hayakawa 2004, Koizumi et al. 2003, Belousova et al. 2002, Noureddini and Curiel 2005, Nicklin et al. 2001), and incorporation of an epitope into the HI loop (residue 543 and 544) resulted in potent anti-epitope immunity (Krause et al. 2006). Therefore, an immunodominant CS-derived B cell epitope was initially inserted into the HI loop of the Fiber protein.

Hexon is the most abundant protein of the adenovirus capsid with 720 copies per virion. In the mature virus, Hexon exists as homotrimeric capsomeres which make up the facets of the icosahedral virion (Rux and Burnett 2004). The crystal structures of adenovirus serotypes 2 and 5 (Ad2 and Ad5) Hexons have been solved, revealing a complex molecular architecture (Athapilly et al. 1994, Roberts et al. 1986, Rux and Burnett 2000). The base of each monomeric subunit consists of two beta-barrel motifs that are present in the capsid proteins of many icosahedral viruses. Three long loops (DE1, FG1, and FG2) extend out from the base structure to form the tower region of each molecule (Rux and Burnett 2004). Sequences within these loop domains protrude to the surface of the capsid to form the exterior of the virion. Alignments from different adenovirus serotypes show that the sequences located on the capsid exterior are poorly conserved in both length and amino acid sequence (Crawford-Miksza and Schnurr 1996). Furthermore, it has been shown that the sequences located in these poorly conserved domains, termed hypervariable regions (HVRs), contain the determinants against which serotype-specific antibodies are produced (Top 1975, Rux and Burnett 2000, Top et al. 1971).

Based on early sequence alignments, seven HVRs were identified throughout the Hexon molecule (Crawford-Miksza and Schnurr 1996, Roberts et al 2006). Because the HVRs are poorly conserved between serotypes and do not appear to be involved in maintaining the structural integrity of Hexon, small changes could be made to these domains without affecting the viability of the virus (Rux and Burnett 2000). For example a hexahistidine tag can be inserted into HVR2, HVR3, HVR5, HVR6, and HVR7 without compromising virus viability (Wu et al. 2005). Thus, Hexon HVRs are often used as targets to efficiently induce an antibody response against peptides located in Hexon HVRs (Worgall et al. 2005, Crompton et al. 1994). Due to its poor conservation in length between serotypes and its position on the outermost surface of the adenovirus capsid (Rux and Burnett 2000, Crawford-Miksza and Schnurr 1996), Hexon HVR5 was initially chosen as a site for epitope insertion. Further, the crystal structure of Hexon indicates that HVR5 is a flexible loop on the capsid surface, suggesting that HVR5 can accommodate relatively large peptides without compromising the structural integrity of the capsid (Roberts et al. 1986). Hexon-specific CD4+ and CD8+ epitopes have recently been identified (Leen et al. 2008), and the CD4+ T cell response to adenovirus is focused against conserved residues within the Hexon protein in humans (Onion et al. 2007, Heemskerk et al. 2006).

The adenovirus core is composed of the viral genome and four core proteins. The terminal protein (TP) is covalently linked to the 5′ end of each linear viral DNA strand at two copies per virion. Noncovalently and nonspecifically bound to the viral DNA through arginine-rich portions are three other core proteins mu (μ), V (pV) and VII (pVII). pVII is the major core protein contributing roughly 700-800 copies per virion, and serves as a histone-like center around which viral DNA is wrapped to form nucleosome structures.

Modification of Adenovirus Capsid Proteins to Enhance Humoral Immunity

In some embodiments, circumsporozoite (CS) adenoviral vectors that have an immunodominant CS protein B epitope in an adenovirus capsid protein (inserted in the Hexon or Fiber) are described. The transgene may be under a promoter such as CMV to augment cell-mediated and humoral immune responses to CS protein.

A central repeat region is the conserved structure of CS protein among Plasmodium species, and antibody against this repeat sequence has been shown to have sporozoite neutralizing activity. Examples of a repeat sequence in Plasmodium CS protein are (NANP)ₙ repeat (P. falciparum; SEQ ID NO:60), ANGAGNQPG repeat (P. vivax; SEQ ID NO:63) and NAAG repeat (P. malariae; SEQ ID NO:64), which can be inserted into adenovirus capsid proteins. In some embodiments, four or more (NANP)ₙ repeats (SEQ ID NO:60) of PfCSP may be inserted into HVR1 of adenovirus serotype 5 Hexon. In some embodiments, two, four, six, eight, ten, fourteen, sixteen, eighteen, twenty, twenty-two, twenty-four, twenty-six, or twenty-eight (NANP)ₙ repeats (SEQ ID NO:60; n=2, 4, 6, 8, 10, 12, 14, 16, 18, 20, 22, 24, 26, 28) of PfCSP are inserted into HVR1 of adenovirus serotype 5 Hexon. The (NANP)ₙ repeat sequence may be additionally inserted in the in the HI loop of Fiber.

In some embodiments, immunodominant neutralizing B cell epitopes to CS were mapped to develop improved CS protein adenovirus vaccines. Mice immunized with recombinant P. yoelii CS protein (PyCS) generated high titers against the two major immunodominant B epitopes, QGPGAP (SEQ ID NO:59) and QQPP (SEQ ID NO:65), but the in vitro neutralization assay indicated that the humoral immune response to QGPGAP epitope (SEQ ID NO:59) itself may account for neutralizing activity because the neutralization could be reversed by adding (QGPGAP)₃ peptide (SEQ ID NO:59; n=3) to the medium. In some embodiments, three or more (QGPGAP)ₙ repeats (SEQ ID NO:59) of PyCS are inserted into HVR1 of adenovirus serotype 5 Hexon. In some embodiments, three, four, five, six, seven, eight, nine, ten, eleven, or twelve (QGPGAP)ₙ repeats (SEQ ID NO:59; n=3, 4, 5, 6, 7, 8, 9, 10, 11, 12) of PyCS may be inserted into HVR1 of adenovirus serotype 5 Hexon. The (QGPGAP)ₙ repeat sequence may be additionally inserted in the in the HI loop of Fiber.

The B cell epitope peptide should be presented on the surface of adenovirus virions so that immune system can recognize the epitope efficiently. Such insertion sites could be HVRs of Hexon and Loop structures in Fiber, and different insertion sites can be combined.

Modification of Adenovirus Capsid and Core Proteins to Enhance T Helper Cell Activation

In another embodiment, a CD4+ epitope specific to the transgene used in an adenoviral vector may be incorporated into adenovirus proteins such as pVII, pV and Hexon to augment immunogenicity of the adenoviral-based vaccine. Professional antigen presenting cells (APC) such as dendritic cells (DC) and B cells can uptake particulated pathogens like virus particles via endocytosis and present CD4+ epitopes in the pathogen to CD4+ T cells which acts as helper cells for humoral and/or cellular immune responses. pVII and Hexon may easily be used as adenovirus target proteins to insert antigenic CD4+ peptides because of high copy number of pVII (700-800 copies) and Hexon (720 copies) in one virion.

Modification of Adenovirus Capsid Proteins to Circumvent Existing Adenovirus Immunity

In some embodiments, adenovirus Fiber and Hexon capsid proteins may be modified to insert a B cell or T helper cell epitope to overcome existing immunity to adenovirus and/or enhance the humoral response to an adenovirus vaccine. An estimated 80% of young adults in human population have circulating neutralizing antibodies to adenovirus (Douglas 2007), especially to serotype 5 (Ad5). In studies utilizing adenovirus as a gene therapy vector, it was found that the presence of neutralizing antibodies in animals limits the expression of transgenes delivered by adenovirus. In addition to neutralizing antibodies, CD8+ T cell responses also contributed to the limitation of recombinant gene expression (Yang et al. 1995, Yang et al 1996). Such pre-existing immunity to adenovirus has previously been reported to inhibit the efficacy of a recombinant adenovirus vaccine (Papp et al. 1999) and also reduces immunogenicity of adenovirus-based vaccines in a clinical trial (Priddy et al. 2008).

Hexon is a major target for anti-Ad capsid immune responses (Roy et al. 2005, Wohlfart 1988), and is likely responsible for the potent adjuvant effect of adenovirus, including the induction of CD4+ and CD8+ T cell responses. Therefore, one strategy that has been employed to circumvent pre-existing anti-adenovirus immunity is to replace all or part of the Hexon with a different protein, for example, rare serotypes such as adenovirus 11, 24, 26 and 35. Because Hexon is a major target of anti-adenovirus neutralizing antibody (Youil et al. 2002, Sumida et al. 2005), the entire Hexon or HVRs of Hexon may be swapped with the rare serotypes (Wu et al. 2002, Roberts et al. 2006).

In another strategy as described in one embodiment herein, an adenoviral Hexon may be modified by replacement of HVR1 or HVR5 with an antigenic peptide to circumvent pre-existing anti-adenovirus immunity or anti-adenovirus neutralizing antibody induced by previous vaccination with adenoviral vector. In some embodiments, an antigenic peptide may be an immunogenic epitope of Plasmodium CS protein, and in certain aspects, the epitope may comprise a central repeat sequence, CD4+ epitope sequence or CD8+ epitope sequence.

Repeat administration with an Ad vector of the same serotype is prevented due to anti-Ad immunity following immunization. Therefore, many Ad vaccines impede boosting of the vaccine by preventing expression and presentation of the antigen encoded by the transgene (Yang 1995, Hackett et al. 2000, Harvey et al. 1999, Mastrangeli et al. 1996). The addition of a specific epitope to the Ad capsid, such as those described in the examples below, may reduce or eliminate this impediment according to some embodiments.

The following examples are provided to better illustrate the embodiments and are not to be interpreted as limiting the scope of any claimed embodiment. The extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1: Construction of Capsid-Modified Plasmodium Circumsporozoite Protein Adenovirus Plasmid Vectors and Recombinant Adenovirus Particles

Epitope Mapping

First, an immunodominant neutralizing B cell epitope in PyCS was chosen. Naïve Balb/c mice were immunized with recombinant PyCS-protein with incomplete freund adjuvant three times and the pooled serum was used to determine the critical epitope of neutralizing antibody.

Briefly, in the neutralizing assay, human CD81 expressing HepG2 cells were used as target cells. CD81 is a molecule necessary for malaria parasites to form parasitophorous vacuoles in hepatocytes where they multiply and develop into schizonts (Silvie et al 2006), thus greatly increasing the in vitro infectivity of sporozoites.

In this assay, PyCS synthetic peptides were added to the wells to block peptide specific antibody. The results of epitope mapping indicated that the PyCS central repeat sequence, QGPGAP (SEQ ID NO:59), is a more potent neutralizing epitope in PyCS than QQPP (SEQ ID NO:65). Therefore, insertion of the (QGPGAP)ₙ epitope (SEQ ID NO:59) was used to modify adenovirus capsid proteins, Hexon and/or Fiber.

Construction of Capsid-Modified Plasmid Vectors

Adenovirus shuttle vector pShuttle-CMV (STRATAGENE) was modified by inserting a GFP-expression cassette under the cloning site. First, BsmBI-SacI fragment (pCMV+GFP) and SacI-BsmBI fragment (SV40 poly A signal) of pmaxGFP (Lonza, Cologne, Germany) were blunted and inserted into the blunted SalI and KpnI sites of pUC19 respectively. The BamHI-EcoRI fragment of the resulting pCMV-GFP/pUC19 was inserted into the same sites of SV40 pA/pUC19 to create SV40 pA-pCMV-GFP fragment. The fragment was blunted and inserted into the EcoRV site of pShuttle-CMV. The resulting shuttle vector (GFP/pShuttle-CMV) has dual pCMV promoters and SV40pAs for a transgene and GFP.

Another modification of the Adenovirus shuttle vector pShuttle-CMV was done to replace the CMV promoter region with CMV5 promoter from pQBI-AdCMV5 (QBIOgene). The SgrAI-KpnI fragment of pShuttle-CMV was replaced with the fragment containing the CMV5 promoter sequence and the upstream sequence from CMV promoter in pShuttle-CMV to construct pShuttle-CMV5 vector.

The P. yoelii CS (PyCS) gene was codon-optimized except for the (QGPGAP)ₙ repeats (SEQ ID NO:59) by overlapping PCR reaction based on JCat codon-optimization algorithm (http://www jcat de/).

The PfCSP amino acid sequence of P. falciparum 3D7 strain was used as a template sequence for codon-optimization. Codon-optimization for protein expression in humans was done by Integrated DNA Technologies' (Coralville, Iowa USA) optimization software. DNA fragments that encode whole PfCSP except for the GPI-anchored motif at the C-terminus (FIG. 10; SEQ ID NO:2) were synthesized by Integrated DNA Technologies

Codon-optimized PyCS gene (FIG. 9; SEQ ID NO:1) or PfCSP gene (FIG. 10; SEQ ID NO:2) was inserted into KpnI and HindIII sites of pShuttle-CMV, pShuttle-CMV5, or GFP/pShuttle-CMV. The resulting Plasmodium circumsporozoite protein coding adenovirus shuttle vectors were used for homologous recombination with AdEasy-1 to construct adenovirus genome which has Plasmodium circumsporozoite antigenic gene and intact adenovirus protein coding sequences. Briefly, Plasmodium circumsporozoite protein coding adenovirus shuttle vectors were linearized by PmeI digestion, and E. coli BJ5183 cells were co-transformed with the linearized shuttle vector and pAdEasy-1 vector (Bruna-Romero et al 2003) for homologous recombination.

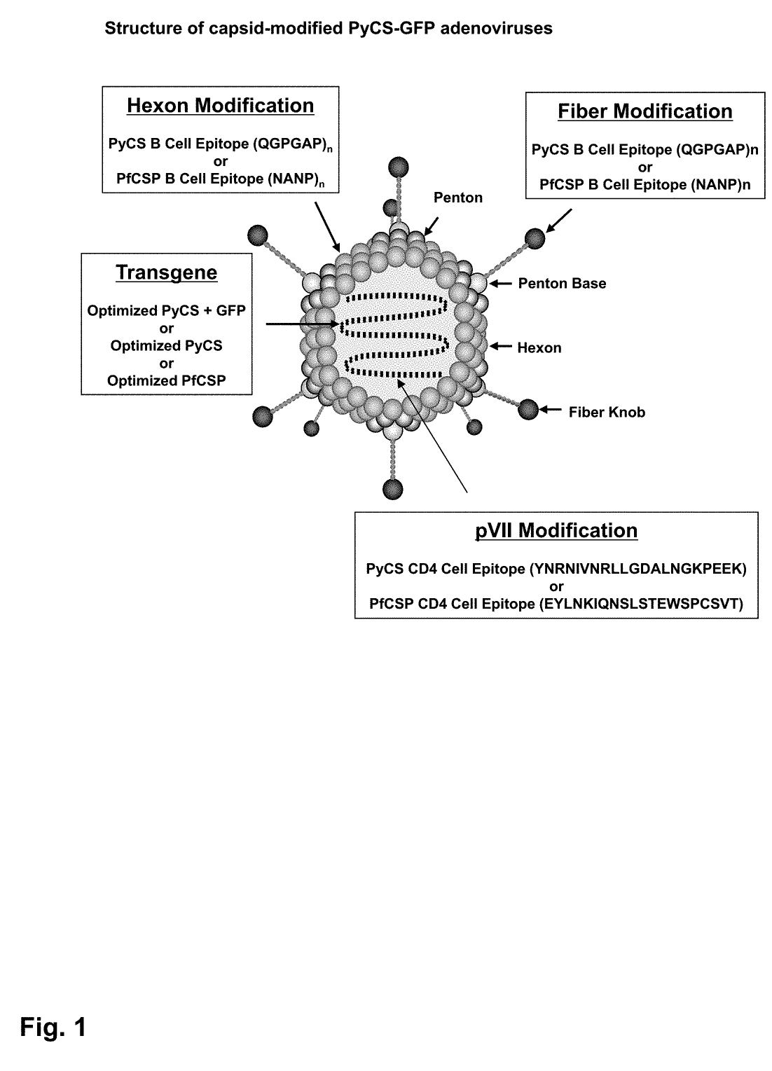

Modification of adenovirus capsid proteins is summarized and illustrated in FIG. 1. Modification of HVR1 sequence in the adenovirus genome DNA is illustrated in FIG. 2. Briefly, AdEasy-1 was digested with SfiI and the 6.4 kbp fragment was subcloned into EcoRI and PstI sites of pUC19 using EcoRI-SfiI and PstI-SfiI linker oligomers. To replace HVR1 with a Plasmodium circumsporozoite protein B cell epitope, the region containing AgeI and NdeI sites was amplified by two-step PCR using primers which have the epitope sequence instead of HVR1 sequence. The PCR product was digested with AgeI and NdeI, and then used to replace the native AgeI-NdeI region of SfiI fragment in SfiI/pUC19 vector. After confirming the sequence, the SfiI fragment of adenovirus genome DNA was replaced with the SfiI fragment containing the circumsporozoite epitope sequence to produce an HVR1-modified Hexon. In some embodiments, an HVR-modified Hexon may have a nucleic acid sequence of SEQ ID NO:3 (FIG. 11), SEQ ID NO:4 (FIG. 12), SEQ ID NO:5 (FIG. 13), SEQ ID NO:6 (FIG. 14), SEQ ID NO:7 (FIG. 15), SEQ ID NO:8 (FIG. 16), SEQ ID NO:9 (FIG. 17), SEQ ID NO:10 (FIG. 18), SEQ ID NO:11 (FIG. 19), SEQ ID NO:12 (FIG. 20), SEQ ID NO:13 (FIG. 21), SEQ ID NO:14 (FIG. 22), SEQ ID NO:15 (FIG. 23), SEQ ID NO:16 (FIG. 24), SEQ ID NO:17 (FIG. 25), SEQ ID NO:18 (FIG. 26), SEQ ID NO:19 (FIG. 27), SEQ ID NO:20 (FIG. 28), SEQ ID NO:21 (FIG. 29), SEQ ID NO:22 (FIG. 30), or SEQ ID NO:23 (FIG. 31).

To insert (NANP)₂₈ (SEQ ID NO:60; n=28) in HVR1, a part of the central repeat region of codon-optimized PfCSP was amplified by PCR using primers having hexon-specific sequence at 5′ and NANP-specific sequence at 3′, and the resulting DNA fragment was inserted into the AgeI-NdeI region by second PCR.

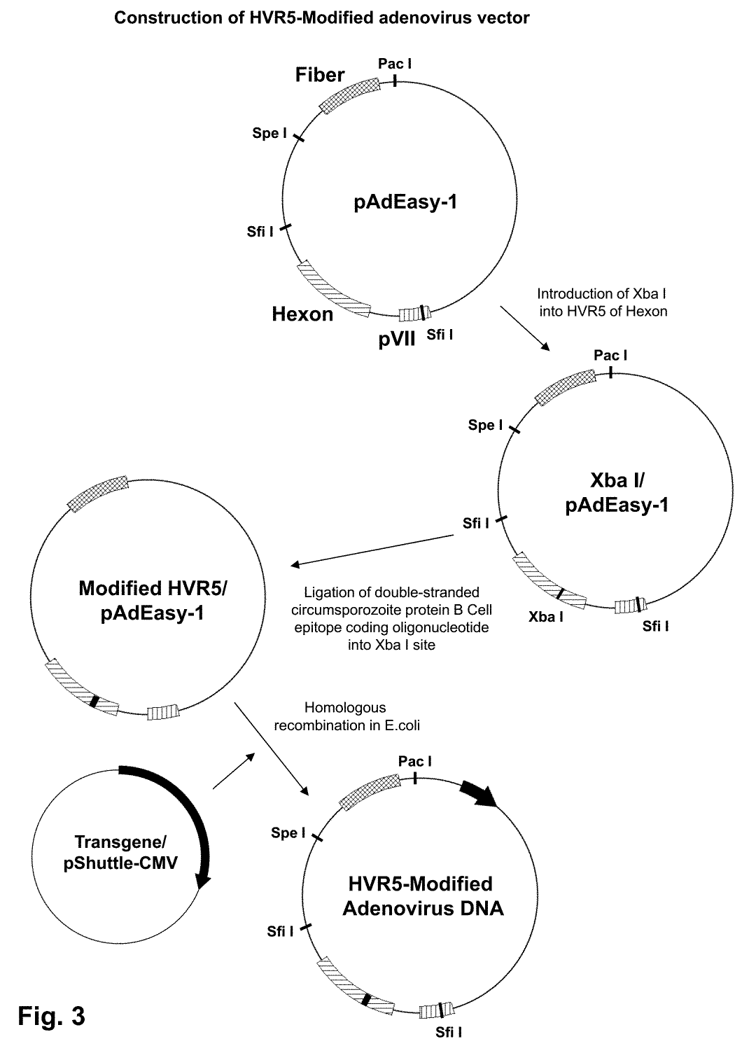

For HVR5-modification, as illustrated in FIG. 3, XbaI site was introduced into HVR5 in the L1 Loop of Hexon in AdEasy-1 and then synthesized, phosphorylated double strand oligomer coding the Plasmodium circumsporozoite protein epitope was inserted into the XbaI site. The insertion was confirmed by sequencing (FIG. 31; SEQ ID NO:23).

For Fiber-modification, as illustrated in FIG. 4, the SpeI-PacI fragment of AdEasy-1 was subcloned into EcoRI and PstI sites of pUC19 using EcoRI-PacI and PstI-SpeI linker oligomers. To insert a Plasmodium circumsporozoite protein B-cell epitope sequence into HI loop of Fiber knob, the region containing EcoNI (or NheI) and MfeI sites was amplified by two-step PCR using primers which have the epitope sequence. The PCR product was digested with EcoNI (or NheI) and MfeI, and then used to replace the native EcoNI (or NheI)-MfeI region of Fiber in SpeI-PacI/pUC19 vector. After confirming the sequence (FIG. 32, SEQ ID NO:24; FIG. 33, SEQ ID NO:25), the SpeI-PacI fragment of AdEasy-1 was replaced with the SpeI-PacI fragment containing the epitope sequence. The resulting Fiber-modified adenovirus DNA was used for homologous recombination with Plasmodium circumsporozoite protein coding adenovirus shuttle vector to produce Fiber-modified Plasmodium circumsporozoite protein adenovirus DNA.

To construct HVR1 and Fiber-modified adenovirus DNA which has two epitope insertions, SfiI-SfiI fragment of Fiber-modified adenovirus DNA was replaced with SfiI-SfiI fragment having the circumsporozoite protein epitope in HVR1 as illustrated in FIG. 5.

To modify the C-terminus of pVII, the region containing Sfi I and Sal I sites was amplified by two-step PCR using primers which have the circumsporozoite protein epitope sequence. The PCR product was digested with SfiI and SalI, and then used to replace the native SfiI-SalI region of SfiI/pUC19 vector (FIGS. 6, 7 and 8). After confirming the sequence (FIG. 34, SEQ ID NO:26; FIG. 35, SEQ ID NO:27), the SfiI-SfiI fragment of HVR1 and/or Fiber-modified circumsporozoite protein adenovirus DNA was replaced with SfiI-SfiI fragment having the circumsporozoite protein epitope in pVII.

To insert the circumsporozoite protein CD4+ epitope sequence EYLNKIQNSLSTEWSPCSVT (SEQ ID NO:62) in the middle of pVII, about 7.7 kb fragment of pAdEasy-1 was prepared by RsrII digestion and cloned between the EcoRI and HindIII sites of pUC19 plasmid using RsrII linker (RsrII/pUC19). The region containing AscI and BglII sites in RsrII/pUC19 was amplified by two-step PCR using primers which have the epitope sequence. The PCR product was digested with AscI and BglII, and then used to replace the native AscI and BglII region in RsrII/pUC19 plasmid. After confirming the sequence of the replaced region (FIG. 36, SEQ ID NO:28; FIG. 37, SEQ ID NO:29), the RsrII fragment of HVR1-modified adenovirus DNA was replaced with the RsrII fragment containing the epitope sequence.

The recombinant adenoviruses listed in Table 1 (P. yoelii) and Table 2 (P. falciparum) below were produced to evaluate the effect of epitope insertion on infectivity, immunogenicity and sensitivity to pre-existing, anti-adenovirus immunity. Recombinant adenovirus vectors used were replication defective, E1 and E3-deleted adenovirus serotype 5 (STRATAGENE). FIG. 1 shows the schematic structure of capsid-modified Plasmodium circumsporozoite protein recombinant adenovirus.

TABLE 1
Recombinant adenoviruses (Plasmodium yoelii circumsporozoite protein)

| | | | | Adeno- | Inser- | | | |
|---|---|---|---|---|---|---|---|---|
| | Recombinant | Pro- | Trans- | virus | tion | | | |
| Antigen | Adenovirus | moter | gene | Protein | Site | Position (a.a.) | Inserted Sequence | Length |
| P. yoelii | wt/Empty | CMV | None | Hexon | — | — | — | — |
| circum- | | | | Fiber | — | — | — | — |
| sporozoite | | | | pVII | — | — | — | — |
| protein | wt/GFP | CMV | GFP | Hexon | — | — | — | — |
| (PyCS) | | | | Fiber | — | — | — | — |
| | | | | pVII | — | — | — | — |
| | wt/PyCS-GFP | CMV | PyCS + | Hexon | — | — | — | — |
| | | | GFP | Fiber | — | — | — | — |
| | | | | pVII | — | — | — | — |
| | (QGPGAP)₃-HVR1/ | CMV | PyCS + | Hexon | HVR1 | from138 to164 | (QGPGAP)₃ | 18 |
| | PyCS-GFP | | GFP | Fiber | — | — | — | — |
| | | | | pVII | — | — | — | — |
| | (QGPGAP)₃-HVR5/ | CMV | PyCS + | Hexon | HVR5 | between 268 and 269 | (QGPGAP)₃ | 18 |
| | PyCS-GFP | | GFP | Fiber | — | — | — | — |
| | | | | pVII | — | — | — | — |
| | (QGPGAP)₃-Fib/ | CMV | PyCS + | Hexon | — | — | — | — |
| | PyCS-GFP | | GFP | Fiber | HI Loop | between 543 and 544 | (QGPGAP)₃ | 18 |
| | | | | pVII | — | — | — | — |
| | (QGPGAP)₃-HVR1/ | CMV | PyCS + | Hexon | HVR1 | from138 to164 | (QGPGAP)₃ | 18 |
| | Fib/PyCS-GFP | | GFP | Fiber | HI Loop | between 543 and 544 | (QGPGAP)₃ | 18 |
| | | | | pVII | — | — | — | — |
| | (QGPGAP)₃-Fib/ | CMV | PyCS + | Hexon | — | — | — | — |
| | PyCD4-pVII-1/ | | GFP | Fiber | HI Loop | between 543 and 544 | (QGPGAP)₃ | 18 |
| | PyCS-GFP | | | pVII | C-terminus | between 198 and | YNRNIVNRLLGDALNGKPEEK | 21 |
| | | | | | | STOP Codon | | |
| | wt/cmv5-PyCS | CMV5 | PyCS | Hexon | — | — | — | — |
| | | | | Fiber | — | — | — | — |
| | | | | pVII | — | — | — | — |
| | (QGPGAP)₃-HVR1/ | CMV5 | PyCS | Hexon | HVR1 | from138 to164 | (QGPGAP)₃ | 18 |
| | cmv5-PyCS | | | Fiber | — | — | — | — |
| | | | | pVII | — | — | — | — |
| | (QGPGAP)₄-HVR1/ | CMV5 | PyCS | Hexon | HVR1 | from138 to164 | (QGPGAP)₄ | 24 |
| | cmv5-PyCS | | | Fiber | — | — | — | — |
| | | | | pVII | — | — | — | — |
| | (QGPGAP)₅-HVR1/ | CMV5 | PyCS | Hexon | HVR1 | from138 to164 | (QGPGAP)₅ | 30 |
| | cmv5-PyCS | | | Fiber | — | — | — | — |
| | | | | pVII | — | — | — | — |
| | (QGPGAP)₆-HVR1/ | CMV5 | PyCS | Hexon | HVR1 | from138 to164 | (QGPGAP)₆ | 36 |
| | cmv5-PyCS | | | Fiber | — | — | — | — |
| | | | | pVII | — | — | — | — |
| | (QGPGAP)₇-HVR1/ | CMV5 | PyCS | Hexon | HVR1 | from138 to164 | (QGPGAP)₇ | 42 |
| | cmv5-PyCS | | | Fiber | — | — | — | — |
| | | | | pVII | — | — | — | — |
| | (QGPGAP)₈-HVR1/ | CMV5 | PyCS | Hexon | HVR1 | from138 to164 | (QGPGAP)₈ | 48 |
| | cmv5-PyCS | | | Fiber | — | — | — | — |
| | | | | pVII | — | — | — | — |
| | (QGPGAP)₉-HVR1/ | CMV5 | PyCS | Hexon | HVR1 | from138 to164 | (QGPGAP)₉ | 54 |
| | cmv5-PyCS | | | Fiber | — | — | — | — |
| | | | | pVII | — | — | — | — |
| | (QGPGAP)₁₁-HVR1/ | CMV5 | PyCS | Hexon | HVR1 | from138 to164 | (QGPGAP)₁₁ | 66 |
| | cmv5-PyCS | | | Fiber | — | — | — | — |
| | | | | pVII | — | — | — | — |
| | (QGPGAP)₁₂-HVR1/ | CMV5 | PyCS | Hexon | HVR1 | from138 to164 | (QGPGAP)₁₂ | 72 |
| | cmv5-PyCS | | | Fiber | — | — | — | — |
| | | | | pVII | — | — | — | — |

TABLE 2
Recombinant adenoviruses (Plasmodium falciparum circumsporozoite protein)

| | | | | Adeno- | Inser- | | | |
|---|---|---|---|---|---|---|---|---|
| | Recombinant | Pro- | Trans- | virus | tion | | | |
| Antigen | Adenovirus | moter | gene | Protein | Site | Position (a.a.) | Inserted Sequence | Length |
| P. falciparum | wt/PfCSP | CMV | PfCSP | Hexon | — | — | — | — |
| circum- | | | | Fiber | — | — | — | — |
| sporozoite | | | | pVII | — | — | — | — |
| protein | (NANP)₄-HVR1/ | CMV | PfCSP | Hexon | HVR1 | from138 to164 | (NANP)₄ | 16 |
| (PfCSP) | PfCSP | | | Fiber | — | — | — | |
| | | | | pVII | — | — | — | |
| | (NANP)₄-Fib/ | CMV | PfCSP | Hexon | — | — | — | |
| | PfCSP | | | Fiber | HI Loop | between 543 and 544 | (NANP)₄ | 16 |
| | | | | pVII | — | — | — | |
| | (NANP)₄-HVR1/ | CMV | PfCSP | Hexon | HVR1 | from138 to164 | (NANP)₄ | 16 |
| | Fib/PfCSP | | | Fiber | HI Loop | between 543 and 544 | (NANP)₄ | 16 |
| | | | | pVII | — | — | — | |
| | (NANP)₆-HVR1/ | CMV | PfCSP | Hexon | HVR1 | from138 to164 | (NANP)₆ | 24 |
| | PfCSP | | | Fiber | — | — | — | |
| | | | | pVII | — | — | — | |
| | (NANP)₈-HVR1/ | CMV | PfCSP | Hexon | HVR1 | from138 to164 | (NANP)₈ | 32 |
| | PfCSP | | | Fiber | — | — | — | |
| | | | | pVII | — | — | — | |
| | (NANP)₁₀-HVR1/ | CMV | PfCSP | Hexon | HVR1 | from138 to164 | (NANP)₁₀ | 40 |
| | PfCSP | | | Fiber | — | — | — | |
| | | | | pVII | — | — | — | |
| | (NANP)₁₂-HVR1/ | CMV | PfCSP | Hexon | HVR1 | from138 to164 | (NANP)₁₂ | 48 |
| | PfCSP | | | Fiber | — | — | — | |
| | | | | pVII | — | — | — | |
| | (NANP)₁₄-HVR1/ | CMV | PfCSP | Hexon | HVR1 | from138 to164 | (NANP)₁₄ | 56 |
| | PfCSP | | | Fiber | — | — | — | |
| | | | | pVII | — | — | — | |
| | (NANP)₁₆-HVR1/ | CMV | PfCSP | Hexon | HVR1 | from138 to164 | (NANP)₁₆ | 64 |
| | PfCSP | | | Fiber | — | — | — | |
| | | | | pVII | — | — | — | |
| | (NANP)₁₈-HVR1/ | CMV | PfCSP | Hexon | HVR1 | from138 to164 | (NANP)₁₈ | 72 |
| | PfCSP | | | Fiber | — | — | — | |
| | | | | pVII | — | — | — | |
| | (NANP)₂₀-HVR1/ | CMV | PfCSP | Hexon | HVR1 | from138 to164 | (NANP)₂₀ | 80 |
| | PfCSP | | | Fiber | — | — | — | |
| | | | | pVII | — | — | — | |
| | (NANP)₂₂-HVR1/ | CMV | PfCSP | Hexon | HVR1 | from138 to164 | (NANP)₂₂ | 88 |
| | PfCSP | | | Fiber | — | — | — | |
| | | | | pVII | — | — | — | |
| | wt/cmv5-PfCSP | CMV5 | PfCSP | Hexon | — | — | — | — |
| | | | | Fiber | — | — | — | — |
| | | | | pVII | — | — | — | — |
| | (NANP)₂₂-HVR1/ | CMV5 | PfCSP | Hexon | HVR1 | from138 to164 | (NANP)₂₂ | 88 |
| | cmv5-PfCSP | | | Fiber | — | — | — | |
| | | | | pVII | — | — | — | |
| | (NANP)₂₈-HVR1/ | CMV5 | PfCSP | Hexon | HVR1 | from138 to164 | (NANP)₁₇(NVDP)₁(NANP)₁₀ | 112 |
| | cmv5-PfCSP | | | Fiber | — | — | — | |
| | | | | pVII | — | — | — | |
| | (NANP)₄-HVR1/ | CMV | PfCSP | Hexon | HVR1 | from138 to164 | (NANP)₄ | 16 |
| | PfCD4-pVII- | | | Fiber | — | — | — | |
| | 1/PfCSP | | | pVII | C-terminus | between 198 and | EYLNKIQNSLSTEWSPCSVT | 20 |
| | | | | | | STOP Codon | | |
| | (NANP)₄-Fib/ | CMV | PfCSP | Hexon | — | — | — | — |
| | PfCD4-pVII- | | | Fiber | HI Loop | between 543 and 544 | (NANP)₄ | 16 |
| | 1/PfCSP | | | pVII | C-terminus | between 198 and | EYLNKIQNSLSTEWSPCSVT | 20 |
| | | | | | | STOP Codon | | |
| | (NANP)₄-HVR1/ | CMV | PfCSP | Hexon | HVR1 | from138 to164 | (NANP)₄ | 16 |
| | Fib/PfCD4- | | | Fiber | — | — | — | |
| | pVII-1/PfCSP | | | pVII | C-terminus | between 198 and | EYLNKIQNSLSTEWSPCSVT | 20 |
| | | | | | | STOP Codon | | |
| | (NANP)₄-HVR1/ | CMV | PfCSP | Hexon | HVR1 | from138 to164 | (NANP)₄ | 16 |
| | PfCD4-pVII- | | | Fiber | — | — | — | |
| | 2/PfCSP | | | pVII | Middle | between 92 and | EYLNKIQNSLSTEWSPCSVT | 20 |
| | | | | | | 93 Codon | | |
| | (NANP)₄-HVR1/ | CMV | PfCSP | Hexon | HVR1 | from138 to164 | (NANP)₄ | 16 |
| | PfCD4-pVII- | | | Fiber | — | — | — | |
| | 3/PfCSP | | | pVII | Middle | between 140 and | EYLNKIQNSLSTEWSPCSVT | 20 |
| | | | | | | 141 Codon | | |
| | (NANP)₂₂HVR1/ | CMV5 | PfCSP | Hexon | HVR1 | from138 to164 | (NANP)₂₂ | 88 |
| | PfCD4-pVII- | | | Fiber | — | — | — | |
| | 3/cmv5-PfCSP | | | pVII | Middle | between 140 and | EYLNKIQNSLSTEWSPCSVT | 20 |
| | | | | | | 141 Codon | | |
| | (NANP)₂₈-HVR1/ | CMV5 | PfCSP | Hexon | HVR1 | from138 to164 | (NANP)₁₇(NVDP)₁(NANP)₁₀ | 112 |
| | PfCD4-pVII-3/ | | | Fiber | — | — | — | |
| | cmv5-PfCSP | | | pVII | Middle | between 140 and | EYLNKIQNSLSTEWSPCSVT | 20 |
| | | | | | | 141 Codon | | |

The capsid-modified adenovirus genome DNA plasmid was purified, linearized by PacI digestion, and used for transfection of AD293 cells.

Adenovirus particles were prepared from the transfected AD293 cells by four rounds of freeze/thaw and used for further virus amplification. After the last amplification, adenovirus particles were purified by CsCl gradient centrifugation. The band was then collected and dialyzed against dialysis buffer to remove CsCl. Virus particle (v.p.) was calculated based on O.D. 260 (1 O.D.260=1.25×10¹² v.p./mL) (Bruna-Romero et al. 2003).

During the adenovirus amplification procedure, small differences in adenovirus growth were observed among capsid-modified adenoviruses, demonstrating that adenovirus infectivity and productivity was not adversely affected by the modification.

### Example 2: Plasmodium yoelii Circumsporozoite Protein-Specific Immune Response

Validation of Plasmodium yoelii Recombinant Adenoviruses

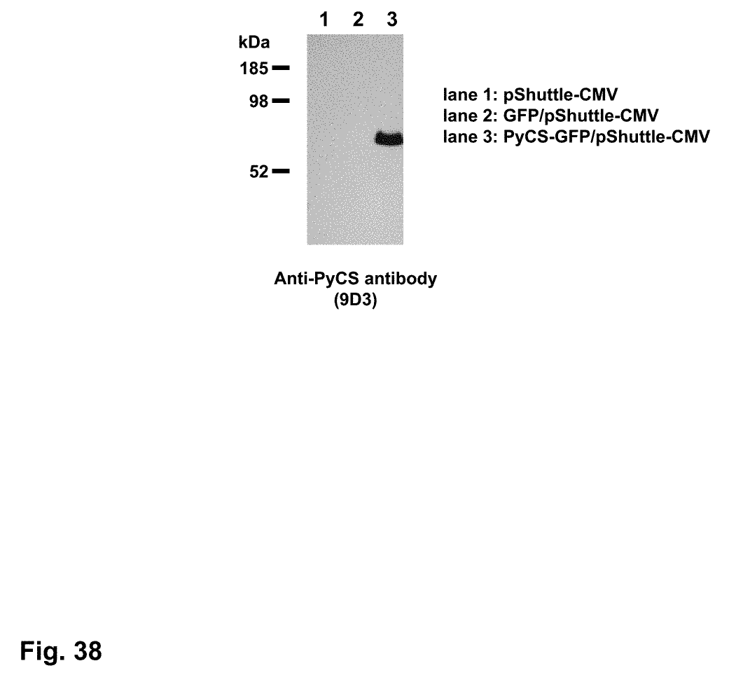

Plasmodium circumsporozoite protein coding adenovirus shuttle vectors were used for transient transfection to confirm Plasmodium circumsporozoite protein expression using AD293 cells (FIG. 38). 24 hours after transfection, cells were lysed in SDS sample buffer followed by SDS PAGE electrophoresis and western blotting with anti-PyCS monoclonal antibody (9D3).

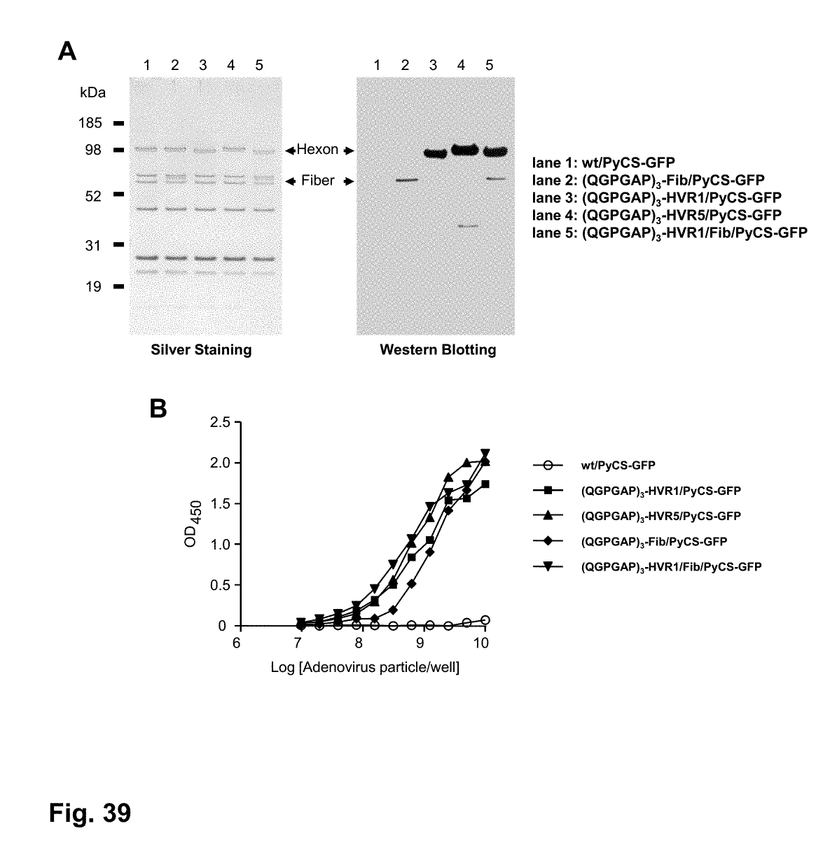

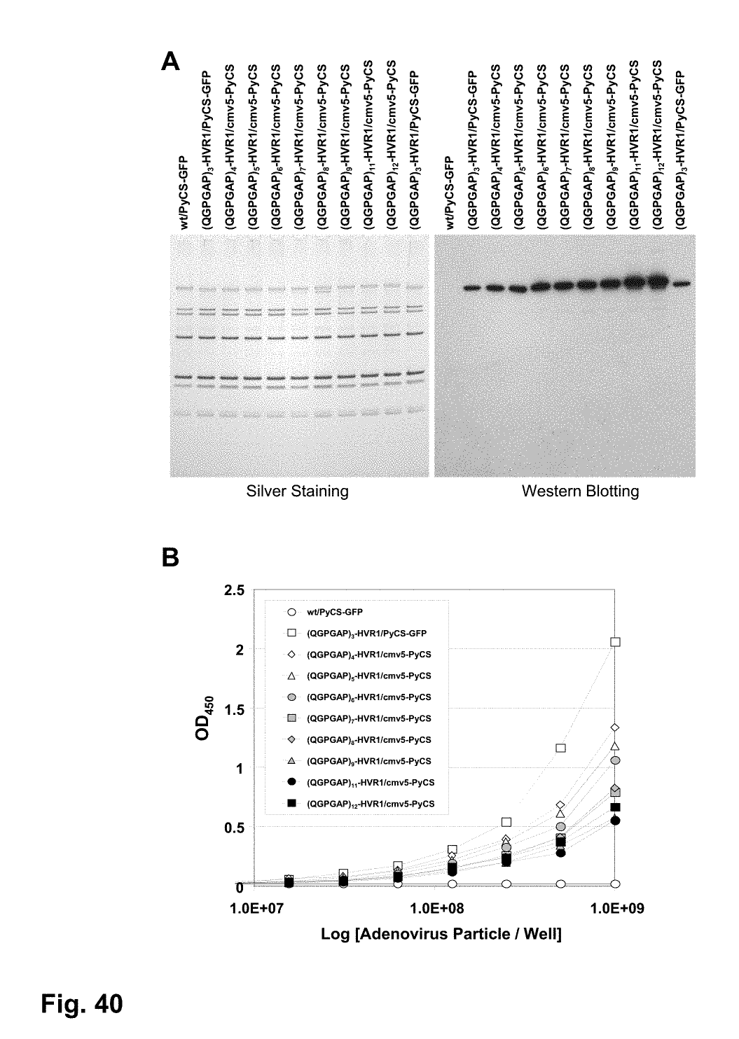

To confirm the epitope insertion into adenovirus capsid proteins, purified recombinant adenoviruses were analyzed by SDS-PAGE (2×10⁹ v.p./lane) and Western blot (1×10⁹ v.p./lane) with anti-sporozoite antibody which recognizes the (QGPGAP)ₙ (SEQ ID NO:59) repeats were done as shown in FIGS. 39A and 40A. The intensity of the bands in FIG. 39A correlated with the copy number of the capsid protein in an adenovirus virion: the copy number of Fiber (36 copies per virion) is twenty-times less than Hexon (720 copies per virion). The lower band in lane 4 in FIG. 39A is likely a degraded Hexon. The intensity of the bands in FIG. 40A correlated with the number of (QGPGAP)ₙ (SEQ ID NO:59) repeats inserted into HVR1.

To assess whether the PyCS-B epitope was exposed to the outside of adenovirus virion, serially diluted purified recombinant adenovirus particles were coated onto Enzyme-Linked Immunosorbent Assay (ELISA) plate and detected with anti-PyCS antibody that recognizes (QGPGAP)ₙ (SEQ ID NO:59) repeats. The antibody recognized all the capsid-modified adenoviruses (FIGS. 39B and 40B). The results of ELISA assay suggest that the PyCS-B epitope incorporated in capsid proteins were well exposed to the outside of adenovirus virions.

Plasmodium yoelii Circumsporozoite Protein-Specific Immune Response after Immunization with Capsid-Modified PyCS Adenovirus

Six- to eight-week old female BALB/c mice were purchased from Taconic (Hudson, N.Y., USA) and maintained under standard conditions in the Laboratory Animal Research Center of The Rockefeller University. For immunization, adenoviruses were diluted in PBS and injected intramuscularly at indicated doses.

To evaluate the immunogenicity of recombinant adenoviruses after a single immunization, groups of naïve BALB/c mice (five per group) were immunized with 1×10⁹v.p. of various recombinant PyCS adenoviruses intramuscularly and PyCS-specific cell-mediated immune responses (CMI) were measured by ELISPOT 2 weeks after immunization (FIG. 41A).

The number of PyCS-specific, IFN-γ-secreting CD8+ T cells in the spleens of immunized mice were determined by an ELISPOT assay, using a synthetic peptide corresponding to the CD8+ T cell epitope (SYVPSAEQI; SEQ ID NO:66) within the PyCS protein. Briefly, 96 well nitrocellulose plates (Milititer HA, Millipore) were coated overnight with anti-mouse interferon γ mAb, R4. After overnight incubation at room temperature, the wells were washed repeatedly with culture medium and blocked with culture medium for 4 hours. 5×10⁵ Splenocytes from immunized mice were added to the ELISPOT wells in the presence or absence of 10 μg/mL CD8+ T cell epitope peptide and incubated 24 hours at 37° C. and 5% CO₂. After extensive washing of the plates with PBS containing 0.05% Tween 20 (PBST), biotinylated anti-mouse interferon γ mAb, XMG1.2, in PBST were added and incubated overnight at 40° C. After washing with PBST, the plates will be incubated with peroxidase-labeled avidin (eBiosciences). The spots were developed by adding AEC substrate (BD Biosciences).

All of the capsid-modified adenoviruses induced comparable level of CMI to adenoviruses having intact capsid protein at this dose (FIG. 41B).

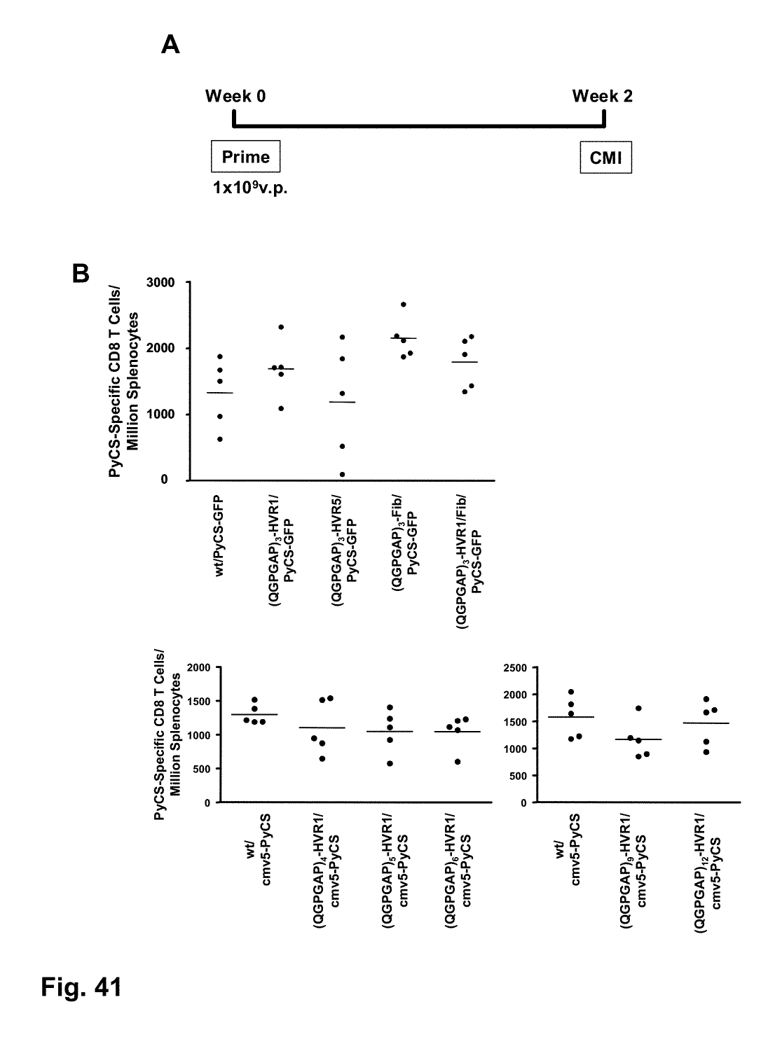

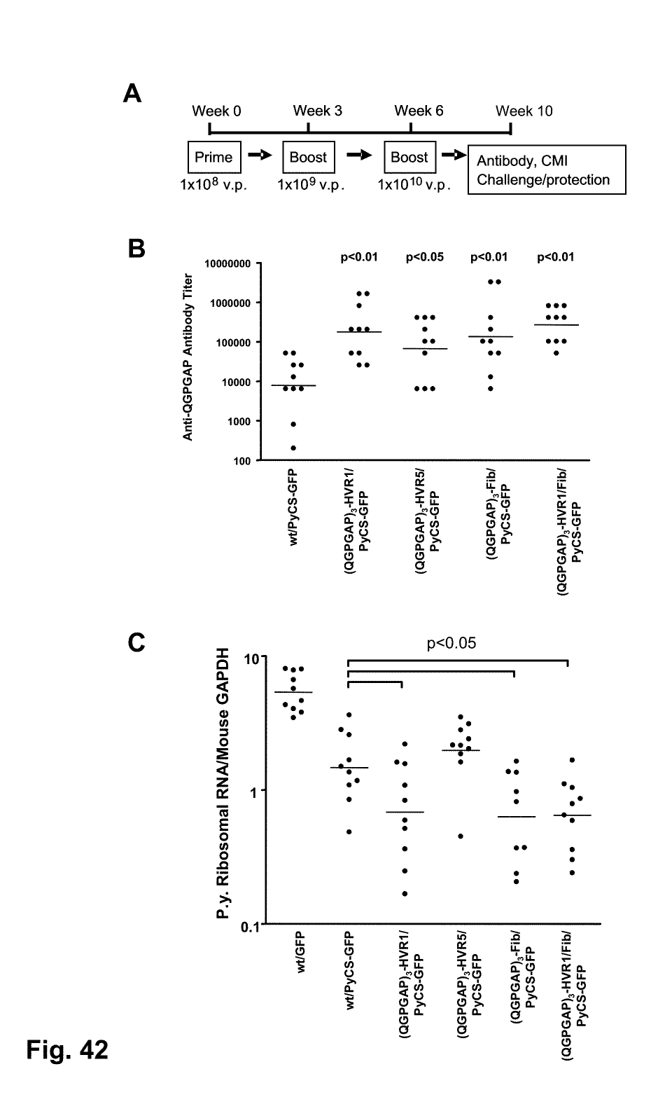

Next, naïve BALB/c mice were given multiple doses of recombinant adenoviruses with increasing doses, i.e. 1×10⁸, 1×10⁹, and 1×10¹⁰ v.p., at 3 week intervals, as shown in FIG. 42A. PyCS-specific humoral response was determined by ELISA. Five microliters of blood was collected from tail vein of the immunized mice and diluted in 495 μl of PBS, and then the samples were centrifuged at 5,000 rpm for 5 min to prepare diluted plasma samples (×100). Maxisorp ELISA plates were coated with 5 μg/ml CS-specific peptide ((QGPGAP)₃; SEQ ID NO:59, n=3) in 0.1M Sodium Carbonate Buffer (pH 9.5) at 4° C. for overnight. Plates were washed and blocked with 1× Diluent for 2 hours at room temperature. The plates were washed again and 100 μl of serially twofold-diluted plasma or serum in 1× Diluent was added to the plates and the plates were incubated for one hour at room temperature. The plates were washed and incubated with 100 μl of HRP-labeled goat anti-mouse IgG antibody. All of the peptides were synthesized by Biosyntheis (Lewisville, Tex., USA).

Using this immunization regimen, all capsid-modified adenoviruses induced a significantly higher level of anti-(QGPGAP)₃ antibody response than wt/PyCS-GFP at week 10 (FIG. 42B).

To determine the vaccine efficacy of capsid-modified adenovirus, the immunized mice were challenged with 2×10⁴ infectious P. yoelii sporozoites via tail vein injection at week 10. Parasite burden 42 hours after sporozoite challenge was determined by quantifying the amounts of parasite-specific ribosomal RNA in mouse liver and described as a ratio of the absolute copy number of parasite ribosomal RNA to that of mouse GAPDH mRNA. For statistical analysis, the values were log-transformed and then one-way ANOVA followed by a Dunnett's test was employed to determine the differences.

Vaccinations with (QGPGAP)₃-HVR1/PyCS-GFP, (QGPGAP)₃-Fib/PyCS-GFP or (QGPGAP)₃-HVR1/Fib/PyCS-GFP induced a higher level of protection than wt/PyCS-GFP, resulting in a significantly lower parasite burden in the malaria challenged mice (FIG. 42C).

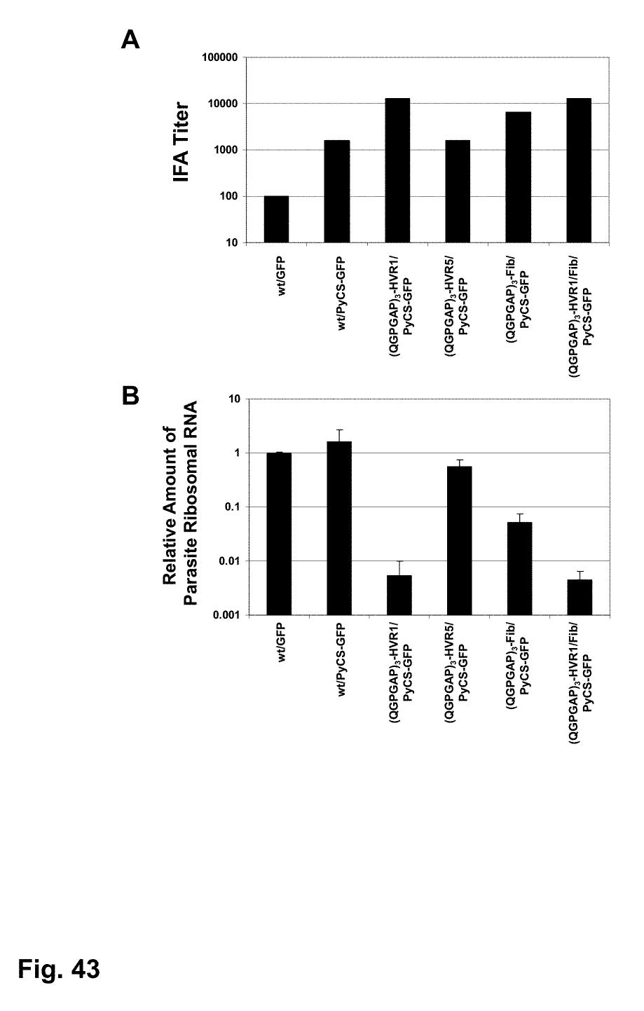

Next, the functionality of PyCS-specific antibody induced by capsid-modified adenoviruses was evaluated. First, to test whether the sera of adenovirus-immunized mice at week 10 (FIG. 42A) could recognize intact sporozoites, an indirect immunofluorescene assay (IFA) was performed. In IFA, air-dried sporozoites on multi-spot glass slides were incubated with 3% Bovine Serum albumin (BSA) in PBS for one hour and then incubated with diluted sera for one hour. After washing, the slides were incubated with fluorescent-labeled secondary antibody for one hour. The slides were washed and IFA titers were determined as the highest dilution producing fluorescence under a fluorescent microscope. Both (QGPGAP)₃-HVR1/PyCS-GFP and (QGPGAP)₃-HVR1/Fib/PyCS-GFP induced a highest IFA titer against sporozoites (FIG. 43A), indicating that the insertion of (QGPGAP)₃ epitope in HVR1 of adenovirus Hexon enabled PyCS adenovirus to elicit a robust antibody response against not only a synthetic peptide, but also a native epitope present in the malaria parasites.

Second, to determine whether mice immunized with capsid-modified adenovirus (FIG. 42A) developed “functional” antibodies that could neutralize the infectivity of sporozoites, an in vitro sporozoite neutralization assay was performed.

In the in vitro neutralizing assay, P. yoelii sporozoites were added to CD81/HepG2 in a 96-well plate in the presence of 30-fold diluted pooled serum from adenovirus-immunized mice. After a two-hour incubation, uninfected sporozoites were washed out with medium and then the cells were cultured for 42 hours. Relative amount of parasite ribosomal RNA to human GAPDH mRNA was measured by real-time PCR (Ophorst et al. 2006).

The pooled serum samples from mice immunized with capsid-modified adenovirus, particularly (QGPGAP)₃-HVR1/PyCS-GFP and (QGPGAP)₃-HVR1/Fib/PyCS-GFP, almost completely inhibited (99%) the sporozoite infectivity in vitro (FIG. 43B). It is noted that the degree of inhibition in this assay was inversely correlated with the IFA titers shown in FIG. 43A.

Protection from Blood Stage Malaria Infection

Next it was determined whether immunization with capsid-modified rAd protects mice from developing a blood-stage malaria infection after sporozoite challenge. The experiments were performed twice and in each experiment, 20 BALB/c mice in each group were immunized three times with wt/PyCS-GFP or (QGPGAP)₃-HVR1/PyCS-GFP as shown in FIG. 42A and at 4 weeks after the last immunization, the mice were intravenously challenged with 50 P. yoelii sporozoites. Giemsa-stained blood smears were analyzed from 3 to 12 days after challenge to detect blood stage malaria parasite infection. In the wt/CS-GFP immunized group, 30 out of 40 mice (75%) were infected whereas 35 out of 40 (87.5%) became infected in the naïve group (Table 3, below). (QGPGAP)₃-HVR1/CS-GFP immunized mice were more protected than wt/CS-GFP; only 15 out of 40 (37.5%) of which became infected, which is consistent with the result of protection experiment measured by parasite burden in liver (FIG. 42C).

TABLE 3
Detection of blood stage malaria parasite infection after
immunization with wt/PyCS-GFP or (QGPGAP)₃-HVR1/PyCS-GFP.

| | | | |
|---|---|---|---|
| | No. of Mice | No. of Mice | Protection |
| Immunization | (Chllenged) | (Infected) | (%) |

Experiment 1

| | | | |
|---|---|---|---|
| None | 20 | 18 | 10 |
| wt/PyCS-GFP | 20 | 14 | 30 |
| (QGPGAP)₃-HVR1/PyCS-GFP | 20 | 10 | 50 |

Experiment 2

| | | | |
|---|---|---|---|
| None | 20 | 17 | 15 |
| wt/PyCS-GFP | 20 | 16 | 20 |
| (QGPGAP)₃-HVR1/PyCS-GFP | 20 | 5 | 75 |

Total

| | | | |
|---|---|---|---|
| None | 40 | 35 | 87.5 |
| wt/PyCS-GFP | 40 | 30 | 75 |
| (QGPGAP)₃-HVR1/PyCS-GFP | 40 | 15 | 37.5 |

Prime-Boost Immunization 1

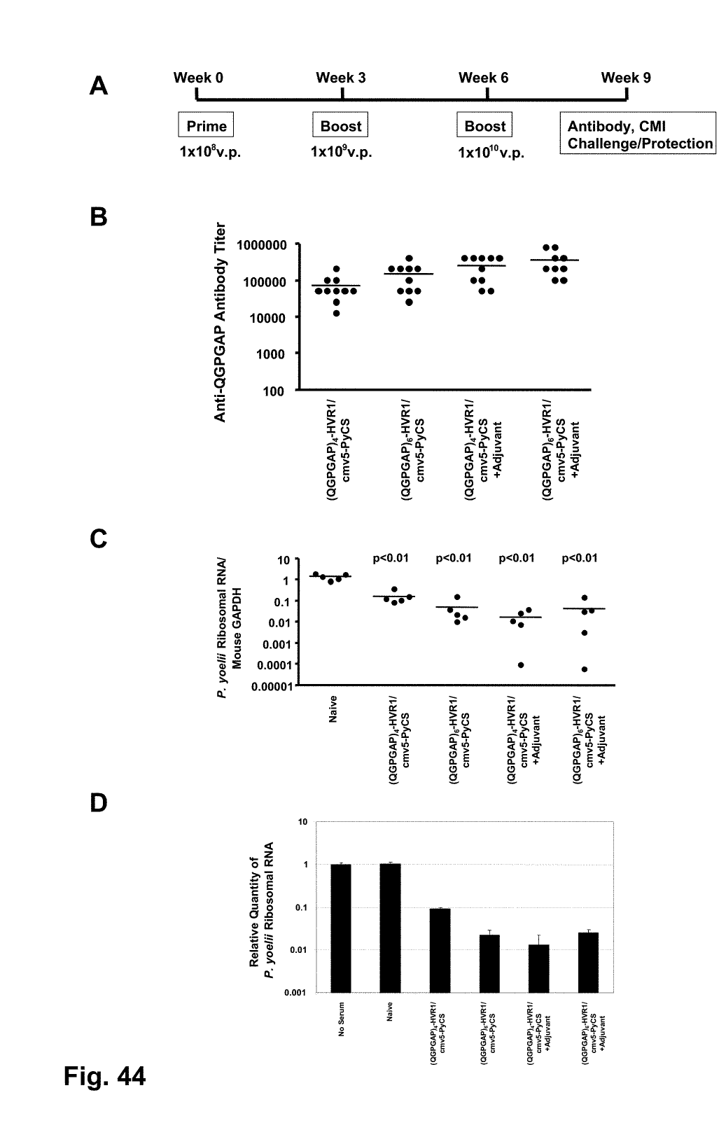

Next, naïve BALB/c mice were given “boosts” of HVR1-modified PyCS adenoviruses which have four or six repeats of (QGPGAP)ₙ (SEQ ID NO:59; n=4, 6) with or without the adjuvant at multiple increasing doses (i.e. 1×10⁸, 1×10⁹, and 1×10¹⁰ v.p.) at 3 week intervals, as shown in FIG. 44A. The adjuvant used in this experiment is Sigma Adjuvant System (Sigma-Aldrich) containing 200 μg/mL Saponin (Sigma-Aldrich). A vial of Sigma Adjuvant System (1 mL) contains 0.5 mg Monophosphoryl Lipid A (detoxified endotoxin) from Salmonella minnesota and 0.5 mg synthetic Trehalose Dicorynomycolate in 2% oil (squalene)-Tween 80 in water. Adenovirus solution was mixed with the equal amount of the adjuvant before the immunization. One hundred microliters of the adenovirus-adjuvant mixture was injected intramuscularly. PyCS-specific humoral and cell-mediated immune responses were measured as described above. A trend was observed that HVR1-modified adenovirus having six repeats induced higher antibody titer than that having four repeats and the use of adjuvant augmented the antibody titer (FIG. 44B). In contrast, there was no effect of the adjuvant on CMI (data not shown).

To determine the vaccine efficacy of HVR1-modified PyCS adenovirus, five mice in each group were challenged with 2×10⁴ infectious P. yoelii sporozoites via tail vein injection at week 9. Parasite burden 42 hours after sporozoite challenge was determined as described above. For statistical analysis, the values were log-transformed and then one-way ANOVA followed by a Dunnett's test was employed to determine the differences. There was a trend that HVR1-modified adenovirus having six repeats reduced parasite burden more than that having four repeats and the use of adjuvant augmented the protection (FIG. 44C).

To evaluate the functionality of antibody induced by HVR1-modified PyCS adenoviruses, we performed an in vitro sporozoite neutralization assay as described above. Pooled serum samples from mice immunized with HVR1-modified PyCS adenoviruses at week 9 neutralized sporozoite invasion at 50-fold dilution (FIG. 44D).

Prime-Boost Immunization 2

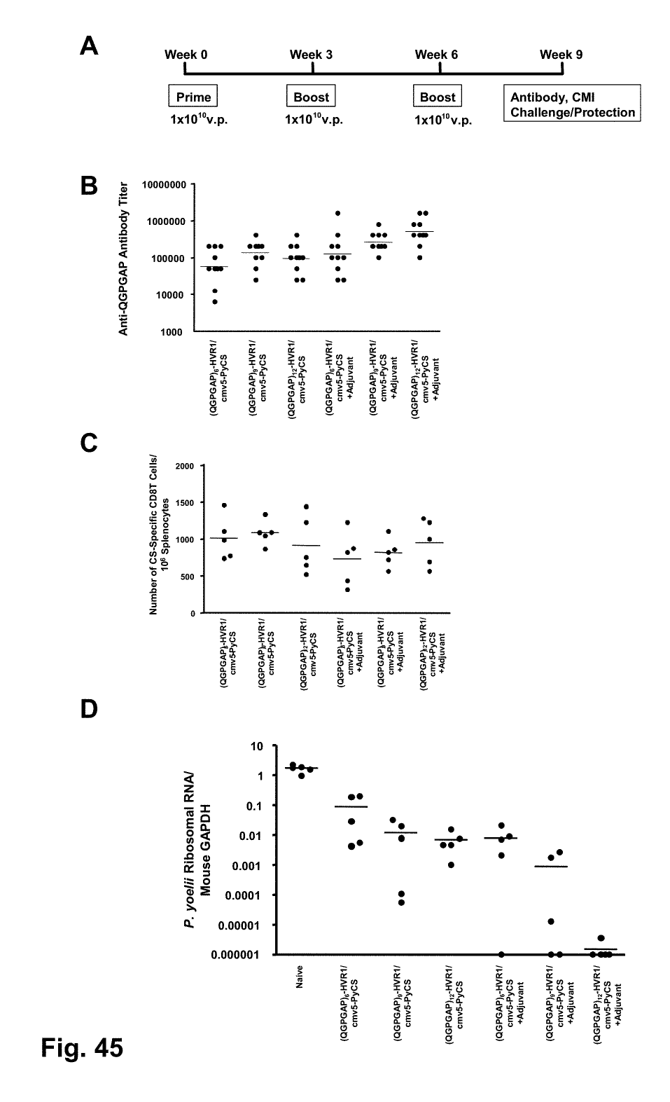

Naïve BALB/c mice were given “boosts’ of HVR1-modified PyCS adenoviruses which have six, nine, or twelve repeats of (QGPGAP)ₙ (SEQ ID NO: 59; n=6, 9, 12) with or without the adjuvant at three doses of 1×10¹⁰ v.p. at 3 week intervals, as shown in FIG. 45A. The adjuvant used in this experiment is Sigma Adjuvant System (Sigma-Aldrich) containing 200 μg/mL Saponin (Sigma-Aldrich). Adenovirus solution was mixed with the equal amount of the adjuvant before the immunization. PyCS-specific humoral and cell-mediated immune responses were measured as described above. HVR1-modified PyCS adenovirus which has twelve repeats of (QGPGAP)ₙ (SEQ ID NO:59; n=12) with the adjuvant induced the highest antibody titer among the groups at week 9 (FIG. 45B). With respect to PyCS-specific CMI, there was no difference among the groups, indicating that the longer epitope insertion up to twelve does not impair adenovirus infectivity in vivo (FIG. 45C). Further, the adjuvant did not affect the ability of adenovirus to induce CMI (FIG. 45C).

To determine the vaccine efficacy of HVR1-modified PyCS adenovirus, five mice in each group were challenged with 2×10⁴ infectious P. yoelii sporozoites via tail vein injection at week 9. Parasite burden 42 hours after sporozoite challenge was determined as described above. All of the HVR-1 modified PyCS adenovirus having (QGPGAP)ₙ repeats (SEQ ID NO:59, n=6, 9, 12), with or without adjuvant, showed increased protection. However, HVR1-modified PyCS adenovirus having twelve repeats of (QGPGAP)ₙ (SEQ ID NO:59, n=12) with the adjuvant showed the best protection (FIG. 45D), which was significantly more protective that any other treatment.

### Example 3: Plasmodium falciparum Circumsporozoite Protein-Specific Immune Response

Validation of Plasmodium falciparum Recombinant Adenoviruses

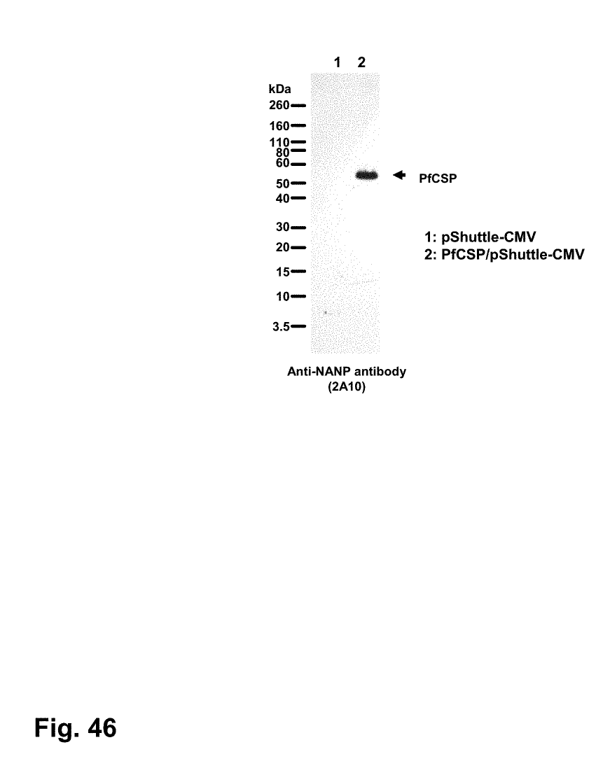

Plasmodium circumsporozoite protein coding adenovirus shuttle vectors were used for transient transfection to confirm Plasmodium circumsporozoite protein expression using AD293 cells (FIG. 46A). 24 hours after transfection, cells were lysed in SDS sample buffer followed by SDS PAGE electrophoresis and western blotting with anti-NANP monoclonal antibody (2A10).

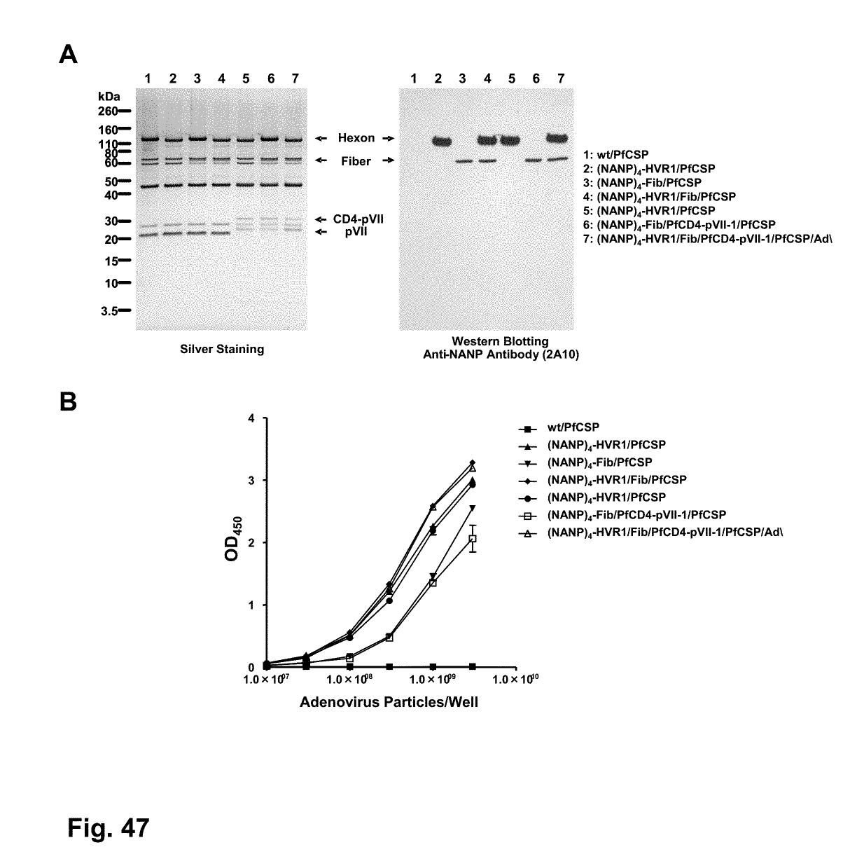

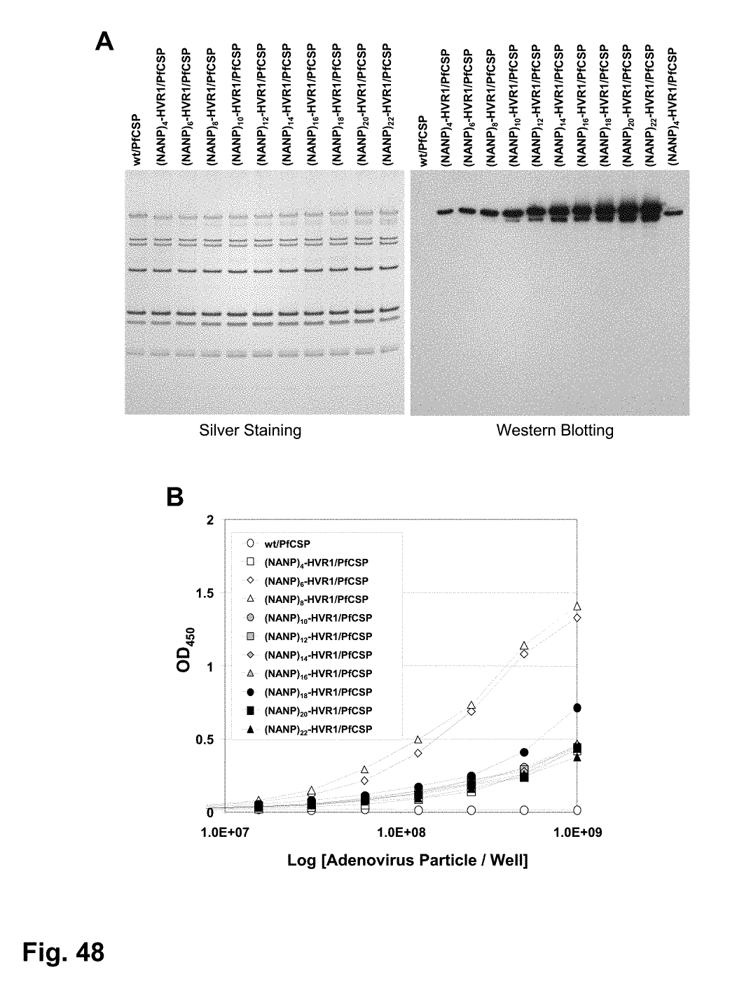

To confirm the epitope insertion into adenovirus capsid proteins, purified recombinant adenoviruses were analyzed by SDS-PAGE (2×10⁹ v.p./lane) and Western blot (1×10⁹ v.p./lane) with anti-sporozoite antibody which recognizes the (NANP)ₙ (SEQ ID NO:60) repeats were done as shown in FIGS. 47A and 48A. The intensity of the bands in FIG. 47A correlated with the copy number of the capsid protein in an adenovirus virion: the copy number of Fiber (36 copies per virion) is twenty-times less than Hexon (720 copies per virion). The intensity of the bands in FIG. 48A correlated with the number of NANP repeat HVR1.

To assess whether the PfCSP-B epitope was exposed to the outside of adenovirus virion, serially diluted purified recombinant adenovirus particles were coated onto Enzyme-Linked Immunosorbent Assay (ELISA) plate and detected with anti-PyCS antibody that recognizes (NANP)ₙ (SEQ ID NO:60) repeats. The antibody recognized all the capsid-modified adenoviruses (FIGS. 47B and 48B). The results of ELISA assay suggest that the PfCSP-B epitope incorporated in capsid proteins were well exposed to the outside of adenovirus virions.

Prime-Boost Immunization 3

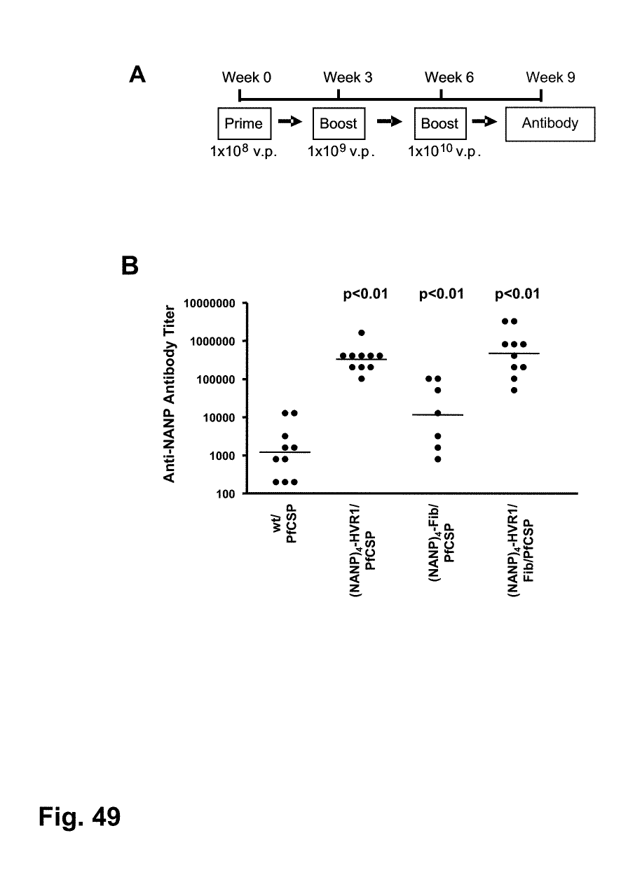

Naïve BALB/c mice were given multiple, increasing doses of recombinant PfCSP adenoviruses (i.e., 1×10⁸, 1×10⁹, and 1×10¹⁰v.p.) at 3 week intervals as shown in FIG. 49A. PfCSP-specific humoral response was determined by ELISA as described above using ELISA plates coated with 1 μg/ml (T1B)₄, a CS repeat peptide which contains a (NANP)ₙ repeat sequence (SEQ ID NO:60) (Calvo-Calle et al 2006). For statistical analysis, the values were log-transformed and one-way ANOVA followed by a Dunnett's test was employed to determine the differences between wt/PfCSP and capsid-modified adenoviruses. All capsid-modified adenoviruses induced statistically higher anti-NANP antibody titer than wt/PfCSP.

Prime-Boost Immunization 4

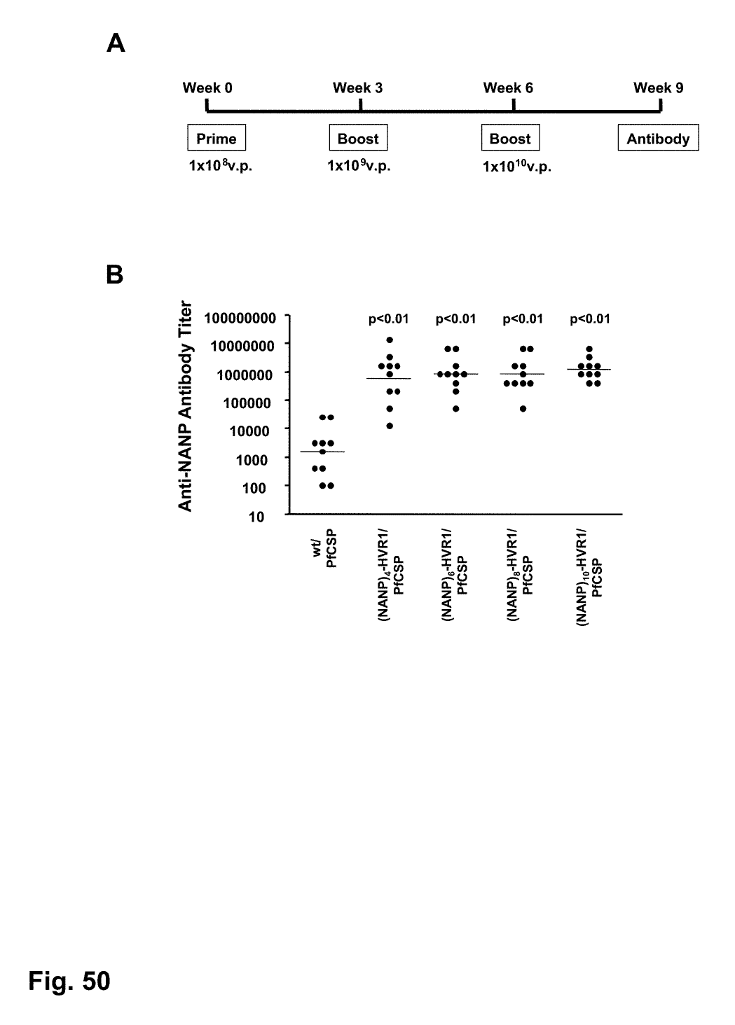

Next, naïve BALB/c mice were given “boosts’ of HVR1-modified PfCSP adenoviruses which have four, six, eight, or ten repeats of (NANP)ₙ (SEQ ID NO:60; n=4, 6, 8, 10) with at multiple increasing doses (i.e., 1×10⁸, 1×10⁹, and 1×10¹⁰v.p.) at 3 week intervals, as shown in FIG. 50A. PfCSP-specific humoral immune responses were measured as described above. All of the HVR1-modified adenoviruses induced significantly higher anti-NANP antibody titer than wt/PfCSP at week 9 (FIG. 50B). For statistical analysis, the values were log-transformed and then one-way ANOVA followed by a Dunnett's test was employed to determine the differences.

Prime-Boost Immunization 5

Naïve BALB/c mice were given “boosts” of HVR1-modified adenoviruses which have ten, sixteen, or twenty-two repeats of (NANP)ₙ (SEQ ID NO:60; n=10, 16, 22) with or without the adjuvant at three doses of 1×10¹⁰ v.p. at 3 week intervals, as shown in FIG. 51A. The adjuvant used in this experiment is Sigma Adjuvant System (Sigma-Aldrich) containing 200 μg/mL Saponin (Sigma-Aldrich). Adenovirus solution was mixed with the equal amount of the adjuvant before the immunization. PfCSP-specific humoral immune response was measured as described above, and it was determined that HVR1-modified adenoviruses with longer B cell epitope induced higher antibody titer (FIG. 51B).

### Example 4: PyCS CD4 Epitope Insertion into Adenovirus Core Protein pVII

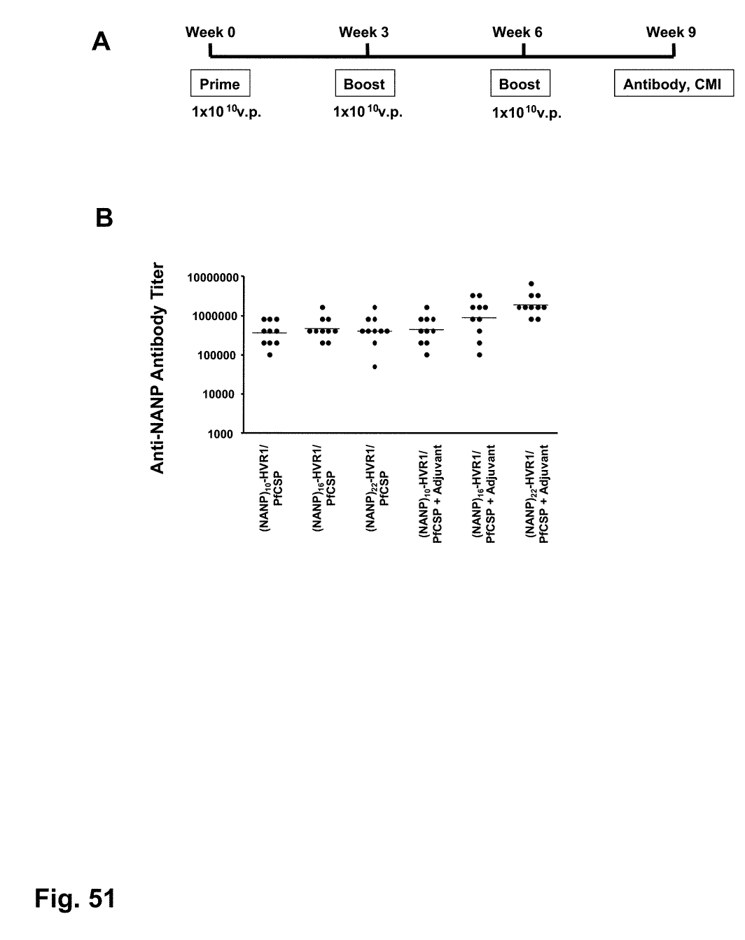

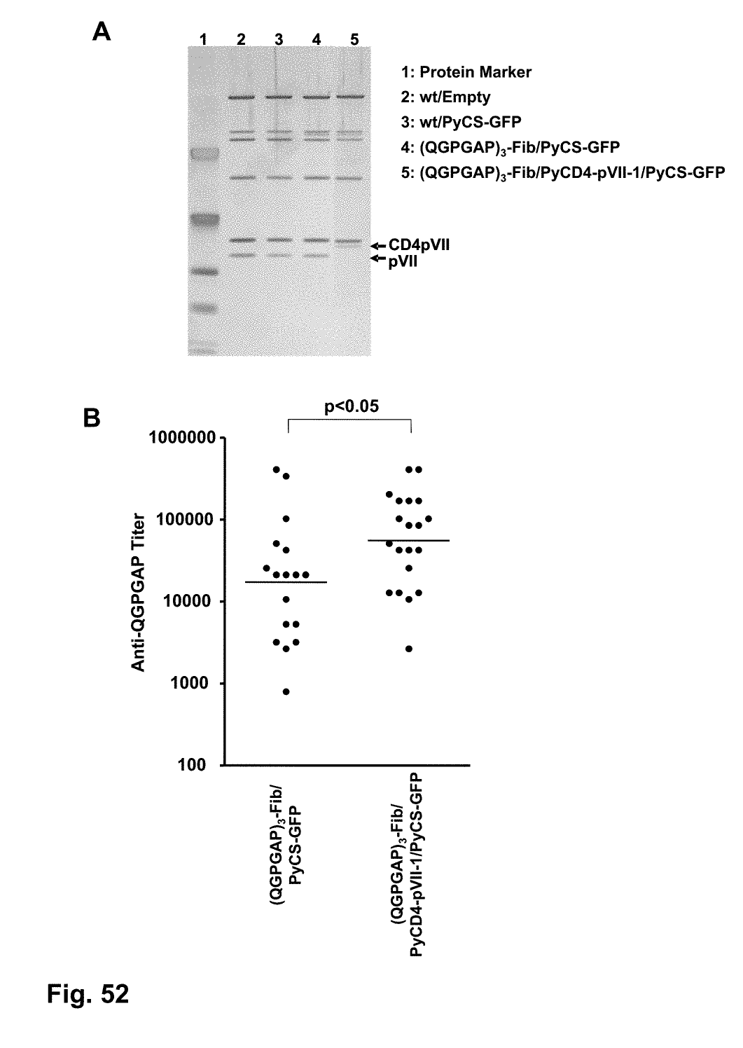

Antigen-specific CD4 T cells are required for antigen-specific B cell development and proliferation. Therefore to determine whether it would be possible to enhance PyCS-specific humoral immune response induced by capsid-modified adenovirus by inserting PyCS CD4 epitope in adenovirus protein, (QGPGAP)₃-Fib/PyCS-GFP that has PyCS CD4 epitope in pVII ((QGPGAP)₃-Fib/CD4-pVII-1/PyCS-GFP) was constructed. pVII is one of the adenovirus core proteins and the copy number per virion is 700-800, which is ideal for efficient CD4 epitope presentation onto MHC class II molecule. As shown in FIG. 52A, the pVII band is shifted by PyCS CD4 epitope insertion into pVII on a SDS-PAGE gel.

To test the effect of PyCS CD4 epitope insertion into pVII, naïve BALB/c mice were immunized with (QGPGAP)₃-Fib/PyCS-GFP or (QGPGAP)₃-Fib/CD4-pVII-1/PyCS-GFP as shown in FIG. 42A and anti-QGPGAP antibody titer was determined by ELISA at week 10. (QGPGAP)₃-Fib/CD4-pVII-1/PyCS-GFP induced significantly higher anti-QGPGAP antibody titer than (QGPGAP)₃-Fib/PyCS-GFP (FIG. 52B), and that indicated PyCS CD4 epitope insertion into pVII augmented humoral immune response induced by capsid-modified adenovirus.

PfCSP CD4 Epitope Insertion into Adenovirus Core Protein pVII

To evaluate the effect of PfCSP CD4+ epitope insertion into different positions in adenovirus core protein pVII on adenovirus-induced immune response, HVR1-modified PfCSP adenoviruses having the PfCSP CD4+ epitope just before the first Nuclear localization Signal (NLS) or between the two NLSs were constructed (FIG. 53A).

To confirm the epitope insertion into pVII, purified recombinant adenoviruses were analyzed by SDS-PAGE as described above. As shown in FIG. 53B, the pVII bands of (NANP)₄-HVR1/CD4-pVII-2/PfCSP and (NANP)₄-HVR1/CD4-pVII-3/PfCSP were shifted upward because of the epitope insertion.

PfCSP-Specific Immune Response Induced by HVR1 and pVII-Modified PfCSP Adenovirus

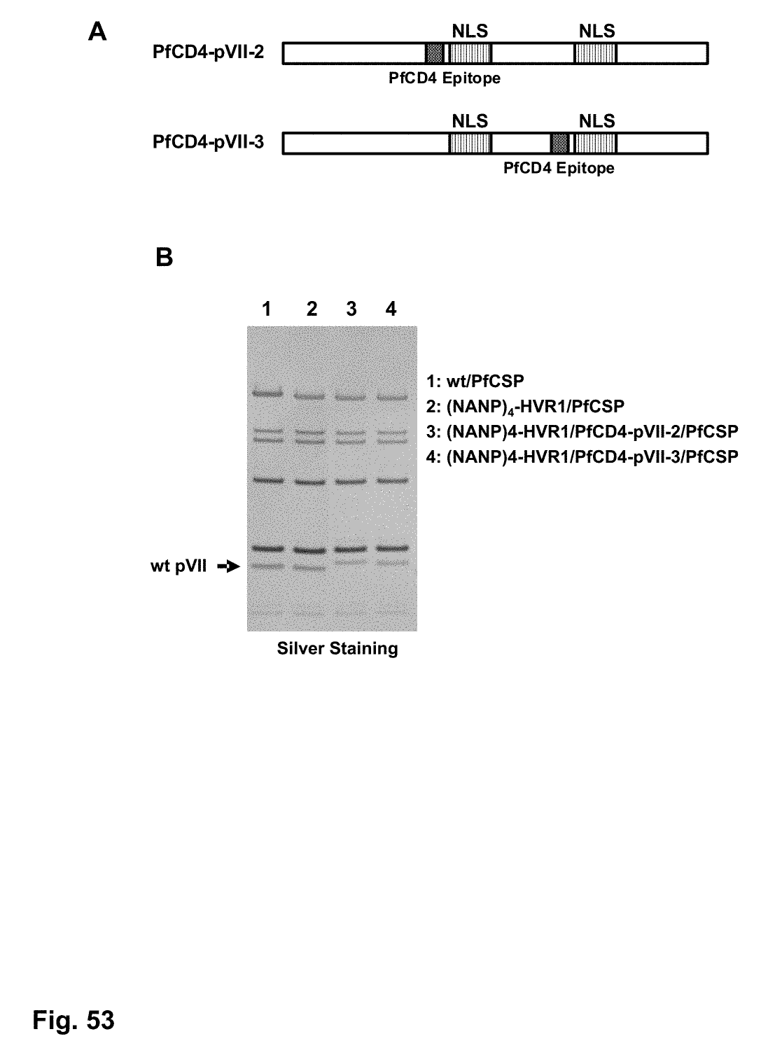

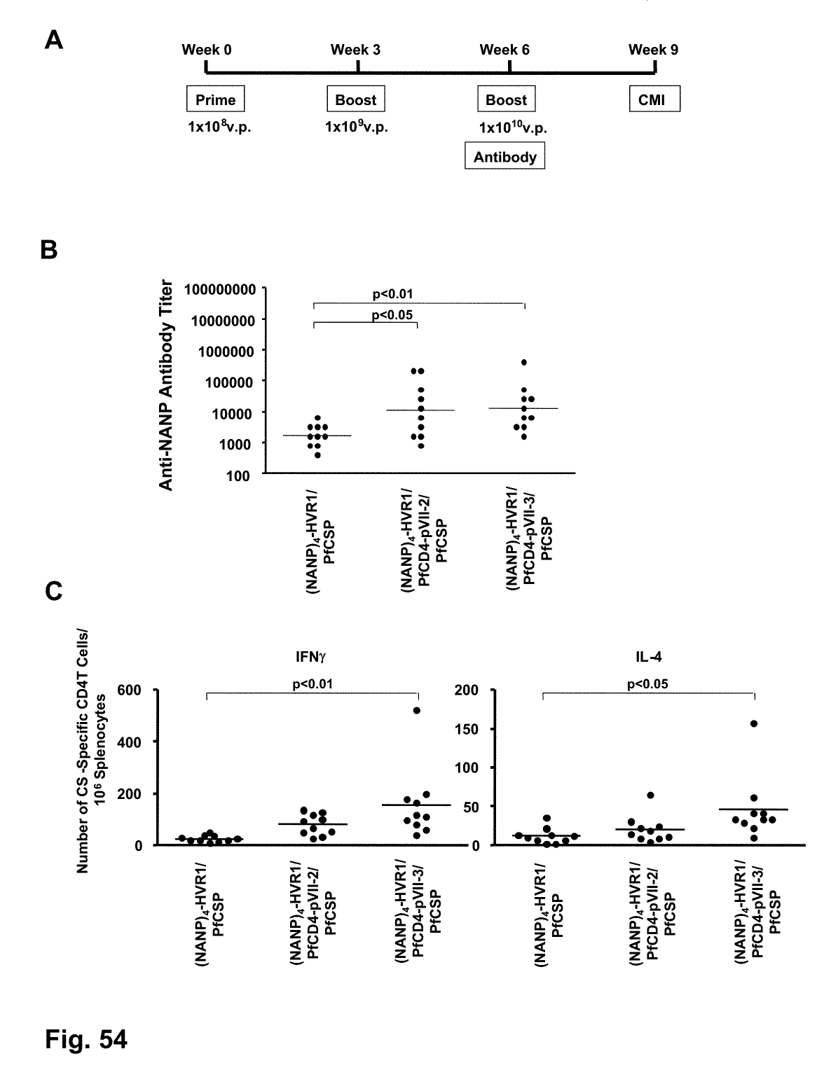

Next, naïve BALB/c mice were given “boosts’ of HVR1 and pVII-modified PfCSP adenoviruses which have four repeats of NANP in HVR1 and the PfCD4+ epitope in pVII with at multiple increasing doses (i.e., 1×10⁸, 1×10⁹, and 1×10¹⁰v.p.) at 3 week intervals, as shown in FIG. 54A. PfCSP-specific humoral immune responses were measured as described above. (NANP)₄-HVR1/CD4-pVII-2/PfCSP and (NANP)₄-HVR1/CD4-pVII-3/PfCSP induced significantly higher anti-NANP antibody titer than (NANP)₄-HVR1/PfCSP at week 6 (FIG. 54B). In terms of CMI, (NANP)₄-HVR1/CD4-pVII-3/PfCSP induced significantly higher IFNγ and IL-4-secreting PfCSP-specific CD4+ T cells than (NANP)₄-HVR1/PfCSP (FIG. 54C).

### Example 5: Effect of Capsid-Modification on Anti-Adenovirus Immunity

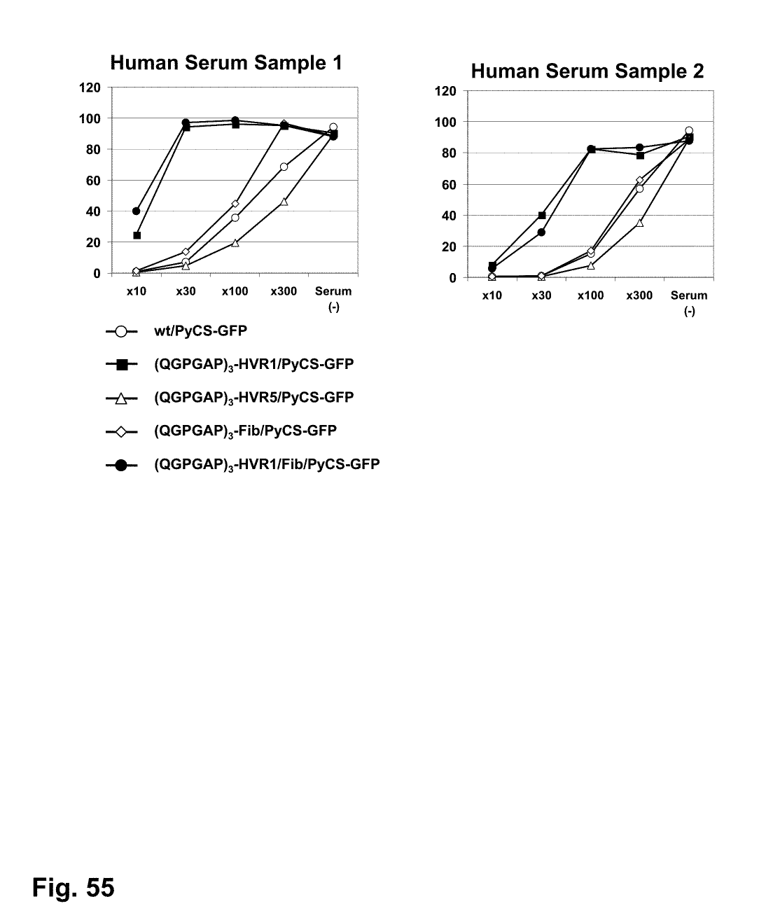

For the in vitro adenovirus neutralization experiments, serum was added to the AD293 cells at the indicated dilutions prior to the adenovirus infection. Caucasian serum samples were obtained from Innovative Research (Novi, Mich., USA). All flow cytometry data was analyzed with FlowJo v8.8 software (Tree Star, Inc, Ashland, Oreg., USA). AD293 cells were infected with each capsid-modified adenovirus in the presence of human adenovirus neutralizing serum samples at the indicated dilution followed by measuring GFP expression by flow cytometry. A replacement of HVR1 with the PyCS-B epitope clearly made the adenovirus resilient to anti-adenovirus serotype 5 sera, whereas the modification of HVR5 or Fiber had no effect (FIG. 55).

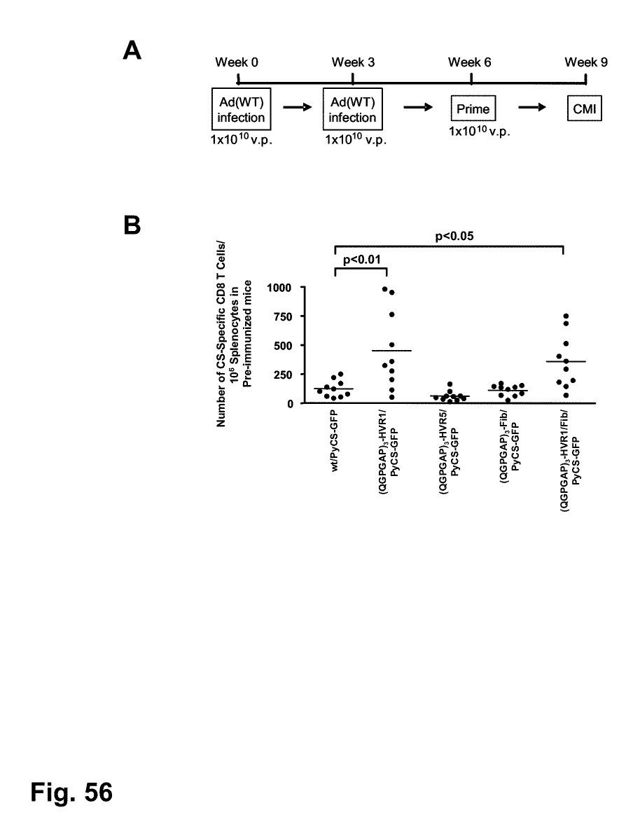

Next, it was determined whether HVR1 is a critical molecule for the neutralization in vivo. For this purpose, mice were infected with 1×10¹⁰ v.p. wt/Empty adenovirus twice to mount sufficient pre-existing anti-adenovirus immunity (FIG. 56A) and randomized based on their anti-adenovirus antibody titers, as determined by ELISA. The mice were then given a single immunizing dose of capsid-modified adenovirus or unmodified adenovirus, and the level of PyCS-specific CD8+ T cell response was measured as described above. Only vaccination with (QGPGAP)₃-HVR1/PyCS-GFP or (QGPGAP)₃-HVR1/Fib/PyCS-GFP was able to induce a significantly more potent CS-specific CD8+ T cell response, compared to that induced by other capsid-modified or unmodified adenovirus (FIG. 56B).

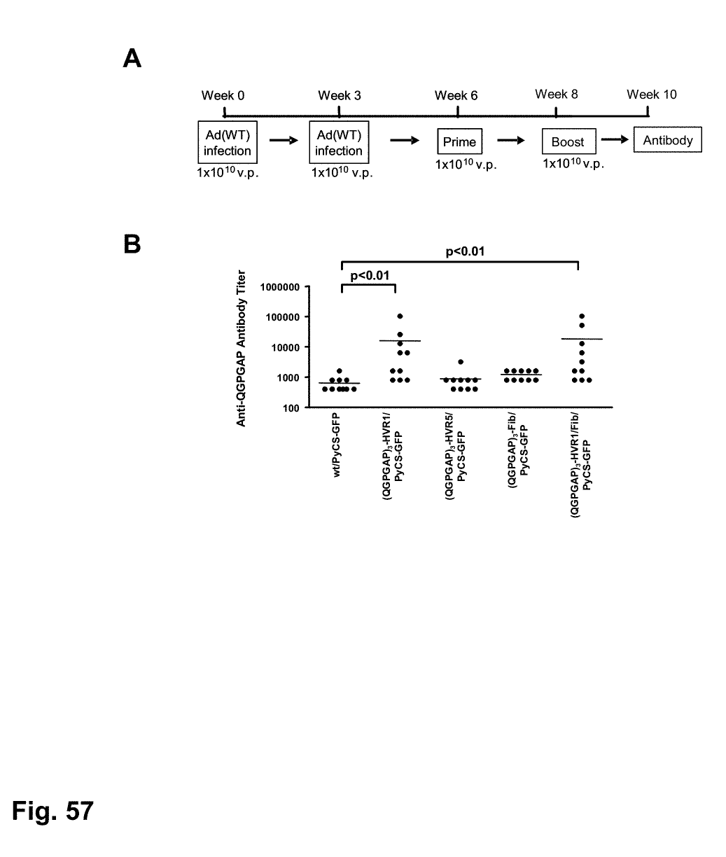

The level of antibody response against (QGPGAP)₃ epitope was also measured, which is expressed on the capsid proteins of rAd, in mice infected with wt/Empty Ad followed by vaccination with capsid-modified rAd (FIG. 57A). Only mice vaccinated with (QGPGAP)₃-HVR1/PyCS-GFP and (QGPGAP)₃-HVR1/Fib/PyCS-GFP were able to mount a significantly higher titer of anti-QGPGAP antibody than those vaccinated with wt/PyCS-GFP (FIG. 57B).

The examples described above are meant to more fully illustrate the embodiments and are not to be interpreted as limiting the scope of any claimed embodiment. In addition, the references cited within the disclosure, and all references listed below are hereby incorporated by reference in their entirety as if fully set forth herein.

### REFERENCES

Abbink, P., Lemckert, A. A., Ewald, B. A., Lynch, D. M., Denholtz, M., et al. 2007. Comparative seroprevalence and immunogenicity of six rare serotype recombinant adenovirus vaccine vectors from subgroups B and D. J Virol. 81:4654-4663.
Alonso, P. L., Sacarlal, J., Aponte, J. J., Leach, A., Macete, E., et al. 2004. Efficacy of the RTS,S/ASO2A vaccine against Plasmodium falciparum infection and disease in young African children: randomised controlled trial. Lancet. 364:1411-1420.
Alonso, P. L., Sacarlal, J., Aponte, J. J., Leach, A., Macete, E., et al. 2005. Duration of protection with RTS,S/ASO2A malaria vaccine in prevention of Plasmodium falciparum disease in Mozambican children: single-blind extended follow-up of a randomised controlled trial. Lancet. 366:2012-2018.
Anderson, R. J., Hannan, C. M., Gilbert, S. C., Laidlaw, S. M., Sheu, E. G., et al. 2004. Enhanced CD8+ T cell immune responses and protection elicited against Plasmodium berghei malaria by prime boost immunization regimens using a novel attenuated fowlpox virus. J Immunol. 172:3094-3100.
Athappilly, F. K., Murali, R., Rux, J. J., Cai, Z. & Burnett, R. M. The refined crystal structure of Hexon, the major coat protein of adenovirus type 2, at 2.9 A resolution. Journal of molecular biology 242, 430-455 (1994).
Barrat, F. J., Meeker, T, Gregorio, J, Chan, J. H., Uematsu, S., et al. Nucleic acids of mammalian origin can act as endogenous ligands for Toll-like receptors and may promote systemic lupus erythematosus. J Exp Med. 202, 1131-1139 (2005)
Bejon, P., Lusingu, J., Olotu, A., Leach, A., Lievens, M., et al. 2008. Efficacy of RTS,S/ASO1E vaccine against malaria in children 5 to 17 months of age. N Engl J Med. 359:2521-2532.
Belousova, N., Krendelchtchikova, V., Curiel, D. T. & Krasnykh, V. Modulation of adenovirus vector tropism via incorporation of polypeptide ligands into the Fiber protein. Journal of virology 76, 8621-8631 (2002).
Bergelson, J. M. et al. Isolation of a common receptor for Coxsackie B viruses and adenoviruses 2 and 5. Science (New York, N.Y. 275, 1320-1323 (1997).
Bewley, M. C., Springer, K., Zhang, Y. B., Freimuth, P. & Flanagan, J. M. Structural analysis of the mechanism of adenovirus binding to its human cellular receptor, CAR. Science (New York, N. Y 286, 1579-1583 (1999).
Bruña-Romero, O., Schmieg, J., Del Val, M., Buschle, M. & Tsuji, M. The dendritic cell-specific chemokine, dendritic cell-derived CC chemokine 1, enhances protective cell-mediated immunity to murine malaria. J Immunol 170, 3195-3203 (2003).Hong, S. S., Karayan, L., Tournier, J., Curiel, D. T. & Boulanger, P. A. Adenovirus type 5 Fiber knob binds to MHC class I alpha2 domain at the surface of human epithelial and B lymphoblastoid cells. The EMBO journal 16, 2294-2306 (1997).
Bruña-Romero, O., Gonzalez-Aseguinolaza, G., Hafalla, J. C., Tsuji, M., and Nussenzweig, R. S. 2001. Complete, long-lasting protection against malaria of mice primed and boosted with two distinct viral vectors expressing the same plasmodial antigen. Proc Natl Acad Sci USA. 98:11491-11496.
Bruna-Romero, O., Rocha, C. D., Tsuji, M. & Gazzinelli, R. T. Enhanced protective immunity against malaria by vaccination with a recombinant adenovirus encoding the circumsporozoite protein of Plasmodium lacking the GPI-anchoring motif. Vaccine 22, 3575-3584 (2004).
Calvo-Calle, J. M., Oliveira, G. A., Watta, C. O., Soverow, J., Parra-Lopez, C., et al. 2006. A linear peptide containing minimal T- and B-cell epitopes of Plasmodium falciparum circumsporozoite protein elicits protection against transgenic sporozoite challenge. Infect Immun. 74:6929-6939.
Clyde, D. F., Most, H., McCarthy, V. C., and Vanderberg, J. P. 1973. Immunization of man against sporozite-induced falciparum malaria. Am J Med Sci. 266:169-177.
Chroboczek, J., Ruigrok, R. W. & Cusack, S. Adenovirus Fiber. Current topics in microbiology and immunology 199 (Pt 1), 163-200 (1995).
Chu, Y., Heistad, D., Cybulsky, M. I. & Davidson, B. L. Vascular cell adhesion molecule-1 augments adenovirus-mediated gene transfer. Arterioscler Thromb Vasc Biol 21, 238-242 (2001).
Crawford-Miksza, L. & Schnurr, D. P. Analysis of 15 adenovirus Hexon proteins reveals the location and structure of seven hypervariable regions containing serotype-specific residues. Journal of virology 70, 1836-1844 (1996).
Crompton, J., Toogood, C. I., Wallis, N. & Hay, R. T. Expression of a foreign epitope on the surface of the adenovirus Hexon. The Journal of general virology 75 (Pt 1), 133-139 (1994).
Crystal, R. G. Transfer of genes to humans: early lessons and obstacles to success. Science (New York, N. Y 270, 404-410 (1995).
Douglas, J. T. Adenoviral vectors for gene therapy. Molecular biotechnology 36, 71-80 (2007).
Edelman, R., Hoffman, S. L., Davis, J. R., Beier, M., Sztein, M. B., et al. 1993. Long-term persistence of sterile immunity in a volunteer immunized with X-irradiated Plasmodium falciparum sporozoites. J Infect Dis. 168:1066-1070.
Grillot, D., Valmori, D., Lambert, P. H., Corradin, G., and Del Giudice, G. 1993. Presentation of T cell epitopes assembled as multiple-antigen peptides to murine and human T lymphocytes. Infect Immun. 61:3064-3067.
Graham, F. L. & Prevec, L. Adenovirus-based expression vectors and recombinant vaccines. Biotechnology (Reading, Mass. 20, 363-390 (1992).
Gwadz, R. W., Cochrane, A. H., Nussenzweig, V., and Nussenzweig, R. S. 1979. Preliminary studies on vaccination of rhesus monkeys with irradiated sporozoites of Plasmodium knowlesi and characterization of surface antigens of these parasites. Bull World Health Organ.57 Suppl 1:165-173.
Hackett, N. R. et al. Use of quantitative TaqMan real-time PCR to track the time-dependent distribution of gene transfer vectors in vivo. Mol Ther 2, 649-656 (2000).
Harvey, B. G. et al. Airway epithelial CFTR mRNA expression in cystic fibrosis patients after repetitive administration of a recombinant adenovirus. The Journal of clinical investigation 104, 1245-1255 (1999).
Heemskerk, B. et al. Adenovirus-specific CD4+ T cell clones recognizing endogenous antigen inhibit viral replication in vitro through cognate interaction. J Immunol 177, 8851-8859 (2006).
Henry, L. J., Xia, D., Wilke, M. E., Deisenhofer, J. & Gerard, R. D. Characterization of the knob domain of the adenovirus type 5 Fiber protein expressed in Escherichia coli. Journal of virology 68, 5239-5246 (1994).
Hong, S. S., Habib, N. A., Franqueville, L., Jensen, S. & Boulanger, P. A. Identification of adenovirus (ad) penton base neutralizing epitopes by use of sera from patients who had received conditionally replicative ad (addl1520) for treatment of liver tumors. Journal of virology 77, 10366-10375 (2003).
Kester, K. E., Cummings, J. F., Ockenhouse, C. F., Nielsen, R., Hall, B. T., et al. 2008. Phase 2a trial of 0, 1, and 3 month and 0, 7, and 28 day immunization schedules of malaria vaccine RTS,S/AS02 in malaria-naïve adults at the Walter Reed Army Institute of Research. Vaccine. 26:2191-2202.
Kirby, I. et al. Mutations in the DG loop of adenovirus type 5 Fiber knob protein abolish highaffinity binding to its cellular receptor CAR. Journal of virology 73, 9508-9514 (1999).
Koizumi, N., Mizuguchi, H., Utoguchi, N., Watanabe, Y. & Hayakawa, T. Generation of Fiber-modified adenovirus vectors containing heterologous peptides in both the HI loop and C terminus of the Fiber knob. The journal of gene medicine 5, 267-276 (2003).
Kozak M. 1987. An analysis of 5′-noncoding sequences from 699 vertebrate messenger RNAs. Nucleic Acids Res. 15:8125-148.
Krause, A., Joh, J. H., Hackett, N. R., Roelvink, P. W., Bruder, J. T., et al. 2006. Epitopes expressed in different adenovirus capsid proteins induce different levels of epitope-specific immunity. J Virol. 80:5523-5530.
Labow, D., Lee, S., Ginsberg, R. J., Crystal, R. G. & Korst, R. J. Adenovirus vector-mediated gene transfer to regional lymph nodes. Human gene therapy 11, 759-769 (2000).
Leen, A. M. et al. Identification of Hexon-specific CD4 and CD8 T cell epitopes for vaccine and immunotherapy. Journal of virology 82, 546-554 (2008).
Leopold, P. L. & Crystal, R. G. Intracellular trafficking of adenovirus: many means to many ends. Advanced drug delivery reviews 59, 810-821 (2007).
Mastrangeli, A. et al. “Sero-switch” adenovirus-mediated in vivo gene transfer: circumvention of anti-adenovirus humoral immune defenses against repeat adenovirus vector administration by changing the adenovirus serotype. Human gene therapy 7, 79-87 (1996).
Mathias, P., Wickham, T., Moore, M. & Nemerow, G. Multiple adenovirus serotypes use alpha v integrins for infection. Journal of virology 68, 6811-6814 (1994).
McConnell, M. J., Danthinne, X., and Imperiale, M. J. 2006. Characterization of a permissive epitope insertion site in adenovirus Hexon. J Virol. 80:5361-5370.
Meier, 0. & Greber, U. F. Adenovirus endocytosis. The journal of gene medicine 5, 451-462 (2003).
Miyazawa, N. et al. Fiber swap between adenovirus subgroups B and C alters intracellular trafficking of adenovirus gene transfer vectors. Journal of virology 73, 6056-6065 (1999).
Miyazawa, N., Crystal, R. G. & Leopold, P. L. Adenovirus serotype 7 retention in a late endosomal compartment prior to cytosol escape is modulated by Fiber protein. Journal of virology 75, 1387-1400 (2001).
Mizuguchi, H. & Hayakawa, T. Targeted adenovirus vectors. Human gene therapy 15, 1034-1044 (2004).
Nakano, M. Y., Boucke, K., Suomalainen, M., Stidwill, R. P. & Greber, U. F. The first step of adenovirus type 2 disassembly occurs at the cell surface, independently of endocytosis and escape to the cytosol. Journal of virology 74, 7085-7095 (2000).
Nicklin, S. A. et al. Ablating adenovirus type 5 Fiber-CAR binding and HI loop insertion of the SIGYPLP peptide generate an endothelial cell-selective adenovirus. Mol Ther 4, 534-542 (2001).
Noureddini, S. C. & Curiel, D. T. Genetic targeting strategies for adenovirus. Molecular pharmaceutics 2, 341-347 (2005).
Nussenzweig, R. S., Vanderberg, J., Most, H., and Orton, C. 1967. Protective immunity produced by the injection of x-irradiated sporozoites of plasmodium berghei. Nature. 216:160-162.
Nussenzweig, R. S. & Long, C. A. Malaria vaccines: multiple targets. Science (New York, N. Y 265, 1381-1383 (1994).
Onion, D. et al. The CD4+ T cell response to adenovirus is focused against conserved residues within the Hexon protein. The Journal of general virology 88, 2417-2425 (2007).
Ophorst, O. J., Radosević, K., Havenga, M. J., Pau, M. G., Holterman, L., et al. 2006. Immunogenicity and protection of a recombinant human adenovirus serotype 35-based malaria vaccine against Plasmodium yoelii in mice. Infect Immun. 74:313-320.
Oualikene, W., Gonin, P. & Eloit, M. Short and long term dissemination of deletion mutants of adenovirus in permissive (cotton rat) and non-permissive (mouse) species. The Journal of general virology 75 (Pt 10), 2765-2768 (1994).
Priddy, F. H., Brown, D., Kublin, J., Monahan, K., Wright, D. P., et al. 2008. Safety and immunogenicity of a replication-incompetent adenovirus type 5 HIV-1 clade B gag/pol/nef vaccine in healthy adults. Clin Infect Dis. 46:1769-1781.
Roberts, M. M., White, J. L., Grutter, M. G. & Burnett, R. M. Three-dimensional structure of the adenovirus major coat protein Hexon. Science (New York, N. Y 232, 1148-1151 (1986).
Roberts, D. M., Nanda, A., Havenga, M. J., Abbink, P., Lynch, D. M., et al. 2006. Hexon-chimaeric adenovirus serotype 5 vectors circumvent pre-existing anti-vector immunity. Nature. 441:239-243.
Rodrigues, E. G., Zavala, F., Eichinger, D., Wilson, J. M., and Tsuji, M. 1997. Single immunizing dose of recombinant adenovirus efficiently induces CD8+ T cell-mediated protective immunity against malaria. J Immunol. 158:1268-1274.
Rodrigues, E. G., Zavala, F., Nussenzweig, R. S., Wilson, J. M. & Tsuji, M. Efficient induction of protective anti-malaria immunity by recombinant adenovirus. Vaccine 16, 1812-1817 (1998).
Roelvink, P. W., Mi Lee, G., Einfeld, D. A., Kovesdi, I. & Wickham, T. J. Identification of a conserved receptor-binding site on the Fiber proteins of CAR-recognizing adenoviridae. Science (New York, N. Y 286, 1568-1571 (1999).
Rux, J. J. & Burnett, R. M. Type-specific epitope locations revealed by X-ray crystallographic study of adenovirus type 5 Hexon. Mol Ther 1, 18-30 (2000).
Rux, J. J. & Burnett, R. M. Adenovirus structure. Human gene therapy 15, 1167-1176 (2004).
Roy, S. et al. Use of chimeric adenoviral vectors to assess capsid neutralization determinants. Virology 333, 207-214 (2005).
Silvie, O., Greco, C., Franetich, J. F., Dubart-Kupperschmitt, A., Hannoun, L., et al. 2006. Expression of human CD81 differently affects host cell susceptibility to malaria sporozoites depending on the Plasmodium species. Cell Microbiol. 8:1134-1146.
Sumida, S. M., Truitt, D. M., Lemckert, A. A., Vogels, R., Custers, J. H., et al. 2005. Neutralizing antibodies to adenovirus serotype 5 vaccine vectors are directed primarily against the adenovirus Hexon protein. J Immunol. 174:7179-7185.
Sun, P., Schwenk, R., White, K., Stoute, J. A., Cohen, J., et al. 2003. Protective immunity induced with malaria vaccine, RTS,S, is linked to Plasmodium falciparum circumsporozoite protein-specific CD4+ and CD8+ T cells producing IFN-gamma. J Immunol. 171:6961-6967.
Tao, D., Barba-Spaeth, G., Rai, U., Nussenzweig, V., Rice, C. M., and Nussenzweig, R. S. 2005. Yellow fever 17D as a vaccine vector for microbial CTL epitopes: protection in a rodent malaria model. J Exp Med. 201:201-209.
Teramoto, S. et al. Investigation of effects of anesthesia and age on aspiration in mice through LacZ gene transfer by recombinant E1-deleted adenovirus vectors. American journal of respiratory and critical care medicine 158, 1914-1919 (1998).
Top, F. H., Jr., Dudding, B. A., Russell, P. K. & Buescher, E. L. Control of respiratory disease in recruits with types 4 and 7 adenovirus vaccines. American journal of epidemiology 94, 142-146 (1971).
Top, F. H., Jr. Control of adenovirus acute respiratory disease in U. S. Army trainees. Yale J Biol Med 48, 185-195 (1975).
Tsuji, M., Romero, P., Nussenzweig, R. S., and Zavala, F. 1990. CD4+ cytolytic T cell clone confers protection against murine malaria. J Exp Med. 172:1353-1357.
Tsuji, M., Rodrigues, E. G. & Nussenzweig, S. Progress toward a malaria vaccine: efficient induction of protective anti-malaria immunity. Biol Chem 382, 553-570 (2001).
Wickham, T. J., Mathias, P., Cheresh, D. A. & Nemerow, G. R. Integrins alpha v beta 3 and alpha v beta 5 promote adenovirus internalization but not virus attachment. Cell 73, 309-319 (1993).
Wohlfart, C. Neutralization of adenoviruses: kinetics, stoichiometry, and mechanisms. Journal of virology 62, 2321-2328 (1988).
Worgall, S. et al. Modification to the capsid of the adenovirus vector that enhances dendritic cell infection and transgene-specific cellular immune responses. Journal of virology 78, 2572-2580 (2004).
Worgall, S., Krause, A., Rivara, M., Hee, K. K., Vintayen, E. V., et al. 2005. Protection against P. aeruginosa with an adenovirus vector containing an OprF epitope in the capsid. J Clin Invest. 115:1281-1289.
Worgall, S., Krause, A., Qiu, J., Joh, J., Hackett, N. R., and Crystal, R. G. 2007. Protective immunity to pseudomonas aeruginosa induced with a capsid-modified adenovirus expressing P. aeruginosa OprF. J Virol. 81:13801-13808.
Wu, H., Dmitriev, I., Kashentseva, E., Seki, T., Wang, M., et al. 2002. Construction and characterization of adenovirus serotype 5 packaged by serotype 3 Hexon. J Virol. 76:12775-12782.
Wu, H. et al. Identification of sites in adenovirus Hexon for foreign peptide incorporation. Journal of virology 79, 3382-3390 (2005).
Xia, D., Henry, L., Gerard, R. D. & Deisenhofer, J. Structure of the receptor binding domain of adenovirus type 5 Fiber protein. Current topics in microbiology and immunology 199 (Pt 1), 39-46 (1995).
Yang, Y., Li, Q., Ertl, H. C. & Wilson, J. M. Cellular and humoral immune responses to viral antigens create barriers to lung-directed gene therapy with recombinant adenoviruses. Journal of virology 69, 2004-2015 (1995).
Youil, R., Toner, T. J., Su, Q., Chen, M., Tang, A., et al. 2002. Hexon gene switch strategy for the generation of chimeric recombinant adenovirus. Hum Gene Ther. 13: 311-320.

1.-20. (canceled)

21. A method of inducing a cellular and humoral immune response against a Plasmodium circumsporozoite protein in a subject comprising administering to the subject at least one dose of a recombinant adenovirus, wherein said recombinant adenovirus is derived from a recombinant adenovirus plasmid vector, and wherein the recombinant adenovirus plasmid vector comprises a nucleotide sequence encoding:
a Plasmodium circumsporozoite protein, or antigenic portion thereof, operably linked to a heterologous promoter, and
one or more modified capsid or core proteins, wherein an immunogenic epitope of Plasmodium circumsporozoite has been inserted into or replaces at least part of the one or more capsid or core proteins.

22. (canceled)

23. A method of inducing a cellular and humoral immune response against a Plasmodium circumsporozoite protein in a subject lacking a pre-existing neutralizing antibody to an adenovirus serotype, comprising administering to the subject:
a first priming dose of a first recombinant adenovirus, and
a subsequent boosting dose of a second recombinant adenovirus,
wherein the first recombinant adenovirus is derived from a recombinant adenovirus plasmid vector, and wherein the recombinant adenovirus plasmid vector comprises a nucleotide sequence encoding a Plasmodium circumsporozoite protein, or antigenic portion thereof, operably linked to a heterologous promoter, and
wherein the second recombinant adenovirus is derived from a recombinant adenovirus plasmid vector, and wherein the recombinant adenovirus plasmid vector comprises a nucleotide sequence encoding a Plasmodium circumsporozoite protein, or antigenic portion thereof, operably linked to a heterologous promoter, and one or more modified capsid or core proteins, wherein an immunogenic epitope of Plasmodium circumsporozoite has been inserted into or replaces at least part of the one or more capsid or core proteins.

24.-28. (canceled)

29. The method of claim 21, wherein Plasmodium circumsporozoite protein comprises a Plasmodium falciparum or a Plasmodium yoelii circumsporozoite protein.

30. The method of claim 21, wherein the nucleotide sequence encoding the Plasmodium circumsporozoite protein comprises a codon-optimized Plasmodium falciparum or Plasmodium yoelii circumsporozoite protein gene encoded by SEQ ID NO:1 or SEQ ID NO:2.

31. The method of claim 21, wherein the immunogenic epitope sequence comprises a B cell and/or a T cell epitope sequence of Plasmodium circumsporozoite protein gene.

32. The method of claim 21, wherein the capsid protein comprises a Hexon hypervariable region (HVR), which comprises HVR1 or HVR5 and the B cell epitope:
a) is inserted in the HVR1 or HVR5 sequence; or
b) replaces a portion of the HVR1 or HRV5 sequence.

33. The method of claim 21, wherein the capsid protein comprises a capsid Fiber protein and the B cell epitope sequence is inserted into the Fiber protein gene.

34. The method of claim 33, wherein the modified capsid protein is encoded by SEQ ID NO:24 or SEQ ID NO:25.

35. The method of claim 31, wherein the B cell epitope sequence is (NANP)₄, (NANP)₆, (NANP)₈, (NANP)₁₀, (NANP)₁₂, (NANP)₁₄, (NANP)₁₆, (NANP)₁₈, (NANP)₂₀, (NANP)₂₂ or (NANP)₂₈.

36. The method of claim 31, wherein the T cell epitope sequence is a CD4+ T cell epitope sequence of Plasmodium circumsporozoite protein gene, and wherein the core protein comprises a pVII protein gene and the CD4+ T cell epitope sequence is inserted into the pVII protein gene.

37. The method of claim 36, wherein the modified core protein gene is encoded by SEQ ID NO:26, SEQ ID NO:27, SEQ ID NO:28 or SEQ ID NO:29, and wherein the CD4+ T cell epitope sequence is EYLNKIQNSLSTEWSPCSVT (SEQ ID NO:62).

38. The method of claim 23, wherein Plasmodium circumsporozoite protein comprises a Plasmodium falciparum or a Plasmodium yoelii circumsporozoite protein.

39. The method of claim 23, wherein the nucleotide sequence encoding the Plasmodium circumsporozoite protein comprises a codon-optimized Plasmodium falciparum or Plasmodium yoelii circumsporozoite protein gene encoded by SEQ ID NO:1 or SEQ ID NO:2.

40. The method of claim 23, wherein the immunogenic epitope sequence comprises a B cell and/or a T cell epitope sequence of Plasmodium circumsporozoite protein gene.

41. The method of claim 23, wherein the capsid protein comprises a Hexon hypervariable region (HVR), which comprises HVR1 or HVR5 and the B cell epitope:
a) is inserted in the HVR1 or HVR5 sequence; or
b) replaces a portion of the HVR1 or HRV5 sequence.

42. The method of claim 23, wherein the capsid protein comprises a capsid Fiber protein and the B cell epitope sequence is inserted into the Fiber protein gene.

43. The method of claim 42, wherein the modified capsid protein is encoded by SEQ ID NO:24 or SEQ ID NO:25.

44. The method of claim 40, wherein the B cell epitope sequence is (NANP)₄, (NANP)₆, (NANP)₈, (NANP)₁₀, (NANP)₁₂, (NANP)₁₄, (NANP)₁₆, (NANP)₁₈, (NANP)₂₀, (NANP)₂₂ or (NANP)₂₈.

45. The method of claim 40, wherein the T cell epitope sequence is a CD4+ T cell epitope sequence of Plasmodium circumsporozoite protein gene, and wherein the core protein comprises a pVII protein gene and the CD4+ T cell epitope sequence is inserted into the pVII protein gene.

46. The method of claim 45, wherein the modified core protein gene is encoded by SEQ ID NO:26, SEQ ID NO:27, SEQ ID NO:28 or SEQ ID NO:29, and wherein the CD4+ T cell epitope sequence is EYLNKIQNSLSTEWSPCSVT (SEQ ID NO:62).


SEQUENCE LISTING (66 sequences)

SEQ ID NO: 1 - DNA, 1127 bp, Artificial Sequence
  Codon-optimized Plasmodium yoelii circumsporozoite protein
  CDS (13)..(1119)
ggtaccgccaccatgaagaagtgcaccatcctggtggtggccagcctgctgctggtggacagcctgctgcccggctacggccagaacaagagcgtgcaggcccagaggaacctgaacgagctgtgctacaacgaggagaacgacaacaagctgtaccacgtgctgaacagcaagaacggcaagatctacaacaggaacatcgtgaacaggctgctgggcgacgccctgaacggcaagcccgaggagaagaaggacgacccccccaaggacgacaacaaggacgacctgcccaaggaggagaagaaggacgacctgcccaaggaggagaagaaggacgacccccccaaggaccccaagaaggacgacccccccaaggaggcccagaacaagctgaaccagcccgtagtggcagatgaaaatgtagatcaagggccaggagcaccacaagggccaggagcaccacaagggccaggagcaccacaggggccaggagcaccacaggggccaggagcaccacaagggccaggagcaccacaaggaccaggagcaccacaagggccaggagcaccacaagggccaggagcaccacaagggccaggagcaccgcaggggccaggagcaccacaagggccaggagcaccacaaggaccaggagcaccacagggtccaggagcaccacaaggaccaggagcaccacaaggaccaggagcaccacaaggtccaggagcaccacaggggccaggagcaccacaagggccaggagcaccacaagaaccaccccaacaacccccacaacagccaccacaacagccaccacaacagccaccacaacagccaccacaacaaccaaacaacaacaacaacaacaacggcaacaacaacgaggacagctacgtgcccagcgccgagcagatcctggagttcgtgaagcagatcagcagccagctgaccgaggagtggagccagtgcagcgtgacctgcggcagcggcgtgagggtgaggaagaggaagaacgtgaacaagcagcccgagaacctgaccctggaggacatcgacaccgagatctgcaagatggacaagtgcagcagcatcttcaacatcgtgagcaacagcctgggctaaagctt

SEQ ID NO: 2 - DNA, 1145 bp, Artificial Sequence
  Codon-optimized Plasmodium falciparus circumsporozoite protein
  CDS (13)..(1134)
ggtaccgccaccatgatgcgcaagctcgccatactgtctgttagtagctttctctttgtagaggccctgtttcaggaataccagtgctatggcagcagcagcaacactcgtgtgctgaacgaacttaactatgataatgcaggaacaaatttatataacgaactggagatgaattactatggtaagcaggaaaattggtactctctgaaaaagaactctagatctctgggcgagaacgacgacggcaataatgaagacaatgaaaagctgaggaagccaaagcacaaaaaactaaagcagcccgcagacggcaatccagaccccaatgctaacccaaacgtggaccccaatgctaatccaaacgtggatcctaacgctaacccgaatgtggaccctaacgccaatccaaatgcgaatcccaacgctaatcctaacgcaaacccgaatgctaaccctaacgcaaaccccaacgctaaccccaacgcgaaccccaatgccaaccccaacgccaacccgaacgccaatccaaacgctaaccctaatgccaatcctaatgccaatccgaacgccaatccaaatgccaatccaaatgctaatcccaacgtggaccccaacgcgaaccctaatgccaaccccaacgctaatccaaatgcgaaccctaacgccaacccgaatgctaatcccaatgccaaccccaatgctaatcccaatgcgaaccctaatgccaatcccaacgccaaccccaacgcaaaccctaatgctaacccgaacgccaatccgaacgcaaatcctaatgctaatcctaacgctaaccccaacgccaatccaaataagaacaatcaaggcaacgggcaggggcacaatatgccaaatgaccctaaccggaatgtcgacgagaatgcaaatgccaatagcgccgtgaaaaataataataacgaggagccaagtgacaaacacattaaggaatatttgaacaagattcaaaactccctctcaacagaatggtctccttgcagcgtgacttgtggaaatggcatccaggttcgtattaaaccaggtagtgccaacaagcccaaggatgaactagactatgcgaatgatatagagaaaaaaatctgtaagatggagaaatgctagcttctaga

SEQ ID NO: 3 - DNA, 2832 bp, Artificial Sequence
  (QGPGAP)3-HVR1 modified Hexon protein
  CDS (1)..(2829)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggccctggagctccacagggaccaggtgcacctcaagggcctggagcccctaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 4 - DNA, 2790 bp, Artificial Sequence
  (QGPGAP)4-HVR1 modified Hexon protein
  CDS (1)..(2787)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccgcaaggtcctggtgctcctaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 5 - DNA, 2808 bp, Artificial Sequence
  (QPGGAP)5-HVR1 modified Hexon protein
  CDS (1)..(2805)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccgcaaggtcctggtgctcctcagggaccaggagcaccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 6 - DNA, 2826 bp, Artificial Sequence
  (QGPGAP)6-HVR1 modified Hexon protein
  CDS (1)..(2823)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccgcaaggtcctggtgctcctcagggaccaggagcaccacagggaccaggagcaccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 7 - DNA, 2844 bp, Artificial Sequence
  (QGPGAP)7-HVR1 modified Hexon protein
  CDS (1)..(2841)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccacagggaccaggagcaccacagggaccaggagcaccacagggaccaggagcaccgcaaggtcctggtgctcctaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 8 - DNA, 2862 bp, Artificial Sequence
  (QGPGAP)8-HVR1 modified Hexon protein
  CDS (1)..(2859)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccacagggaccaggagcaccacagggaccaggagcaccacagggaccaggagcaccgcaaggtcctggtgctcctcagggaccaggagcaccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 9 - DNA, 2880 bp, Artificial Sequence
  (QGPGAP)9-HVR1 modified Hexon protein
  CDS (1)..(2877)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccacagggaccaggagcaccacagggaccaggagcaccacagggaccaggagcaccgcaaggtcctggtgctcctcagggaccaggagcaccacagggaccaggagcaccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 10 - DNA, 2916 bp, Artificial Sequence
  (QGPGAP)11-HVR1 modified Hexon protein
  CDS (1)..(2913)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccacagggaccaggagcaccacagggaccaggagcaccacagggaccaggagcaccacagggaccaggagcaccacagggaccaggagcaccgcaaggtcctggtgctcctcagggaccaggagcaccacagggaccaggagcaccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 11 - DNA, 2934 bp, Artificial Sequence
  (QGPGAP)12-HVR1 modified Hexon protein
  CDS (1)..(2931)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaacagggaccaggagcaccacagggaccaggagcaccacagggaccaggagcaccacagggaccaggagcaccacagggaccaggagcaccacagggaccaggagcaccacagggaccaggagcaccacagggaccaggagcaccgcaaggtcctggtgctcctcagggaccaggagcaccacagggaccaggagcaccacagggaccaggagcaccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcctgcttacccccaacgagtttgaaattaagcgctcagttgacggggagggttacaacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 12 - DNA, 2826 bp, Artificial Sequence
  (NANP)4-HVR1 modified Hexon protein
  CDS (1)..(2823)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaacgctaatcccaacgctaacccaaatgcaaatcctaatgccaaccccaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 13 - DNA, 2790 bp, Artificial Sequence
  (NANP)6-HVR1 modified Hexon protein
  CDS (1)..(2787)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaacgcgaacccgaatgctaatcctaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 14 - DNA, 2814 bp, Artificial Sequence
  (NANP)8-HVR1 modified Hexon protein
  CDS (1)..(2811)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaacgcgaacccgaatgctaatcctaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 15 - DNA, 2838 bp, Artificial Sequence
  (NANP)10-HVR1 modified Hexon protein
  CDS (1)..(2835)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaacgcgaacccgaatgctaatcctaatgcaaatccaaatgcaaatccaaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 16 - DNA, 2862 bp, Artificial Sequence
  (NANP)12-HVR1 modified Hexon protein
  CDS (1)..(2859)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaatgcaaatccaaatgcaaatccaaatgcaaatccaaatgcaaatccaaacgcgaacccgaatgctaatcctaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 17 - DNA, 2886 bp, Artificial Sequence
  (NANP)14-HVR1 modified Hexon protein
  CDS (1)..(2883)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaatgcaaatccaaatgcaaatccaaatgcaaatccaaatgcaaatccaaacgcgaacccgaatgctaatcctaatgcaaatccaaatgcaaatccaaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 18 - DNA, 2910 bp, Artificial Sequence
  (NANP)16-HVR1 modified Hexon protein
  CDS (1)..(2907)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaatgcaaatccaaatgcaaatccaaatgcaaatccaaatgcaaatccaaacgcgaacccgaatgctaatcctaatgcaaatccaaatgcaaatccaaatgcaaatccaaatgcaaatccaaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 19 - DNA, 2934 bp, Artificial Sequence
  (NANP)18-HVR1 modified Hexon protein
  CDS (1)..(2931)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaacgcgaacccgaatgctaatcctaatgcaaatccaaatgcaaatccaaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 20 - DNA, 2958 bp, Artificial Sequence
  (NANP)20-HVR1 modified Hexon protein
  CDS (1)..(2955)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaacgcgaacccgaatgctaatcctaatgcaaatccaaatgcaaatccaaatgcaaatccaaatgcaaatccaaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 21 - DNA, 2982 bp, Artificial Sequence
  (NANP)22-HVR1 modified Hexon protein
  CDS (1)..(2979)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaatgcaaatccaaacgcgaacccgaatgctaatcctaatgcaaatccaaatgcaaatccaaatgcaaatccaaatgcaaatccaaatgcaaatccaaatgcaaatccaaatgcaaatccaaatgcaaatccaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 22 - DNA, 3054 bp, Artificial Sequence
  (NANP)28-HVR1 modified Hexon protein
  CDS (1)..(3051)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaaaacgccaatccaaatgcgaatcccaacgctaatcctaacgcaaacccgaatgctaaccctaacgcaaaccccaacgctaaccccaacgcgaaccccaatgccaaccccaacgccaacccgaacgccaatccaaacgctaaccctaatgccaatcctaatgccaatccgaacgccaatccaaatgccaatccaaatgctaatcccaacaatgcccccaacgcgaaccctaatgccaaccccaacgctaatccaaatgcgaaccctaacgccaacccgaatgctaatcccaatgccaaccccaatgctaatcccaatgcgaaccctaatgccaatcccaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcagcttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 23 - DNA, 2928 bp, Artificial Sequence
  (QGPGAP)3-HVR5 modified Hexon protein
  CDS (1)..(2925)
atggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcagctagccagggccctggagctccacagggaccaggtgcacctcaagggcctggagcccctggcactagcactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataa

SEQ ID NO: 24 - DNA, 1800 bp, Artificial Sequence
  (QGPGAP)3 modified fiber protein
  CDS (1)..(1797)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggacagggccctggagctccacagggaccaggtgcacctcaagggcctggagcccctgacacaactccaagtgcatactctatgtcattttcatgggactggtctggccacaactacattaatgaaatatttgccacatcctcttacactttttcatacattgcccaagaataa

SEQ ID NO: 25 - DNA, 1794 bp, Artificial Sequence
  (NANP)4 modified fiber protein
  CDS (1)..(1791)
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggaaacgctaatcccaacgctaacccaaatgcaaatcctaatgccaaccccgacacaactccaagtgcatactctatgtcattttcatgggactggtctggccacaactacattaatgaaatatttgccacatcctcttacactttttcatacattgcccaagaataa

SEQ ID NO: 26 - DNA, 684 bp, Artificial Sequence
  P. yoelii CD4+ epitope modified pVII sequence
  CDS (1)..(681)
atgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaacgtgcgcacccgccccccgcgcaactacaacaggaacatcgtgaacaggctgctgggcgacgccctgaacggcaagcccgaggagaagtag

SEQ ID NO: 27 - DNA, 657 bp, Artificial Sequence
  P. falciparum CD4+ epitope modified pVII-1 sequence
  CDS (1)..(654)
atgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaacgaatatttgaacaagattcaaaactccctctcaacagaatggtctccttgcagcgtgacttag

SEQ ID NO: 28 - DNA, 741 bp, Artificial Sequence
  P. falciparum CD4+ epitope modified pVII-2 sequence
  CDS (1)..(738)
atgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagccgaatatttgaacaagattcaaaactccctctcaacagaatggtctccttgcagcgtgactcggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaacgtgcgcacccgccccccgcgcaactacaacaggaacatcgtgaacaggctgctgggcgacgccctgacccgccccccgcgcaactag

SEQ ID NO: 29 - DNA, 681 bp, Artificial Sequence
  P. falciparum CD4+ epitope modified PVII-3 sequence
  CDS (1)..(678)
atgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctgaatatttgaacaagattcaaaactccctctcaacagaatggtctccttgcagcgtgactcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaacgtgcgcacccgccccccgcgcaactag

SEQ ID NO: 30 - protein, 369 aa, Artificial Sequence
  Synthetic Construct
MKKCTILVVASLLLVDSLLPGYGQNKSVQAQRNLNELCYNEENDNKLYHVLNSKNGKIYNRNIVNRLLGDALNGKPEEKKDDPPKDDNKDDLPKEEKKDDLPKEEKKDDPPKDPKKDDPPKEAQNKLNQPVVADENVDQGPGAPQGPGAPQGPGAPQGPGAPQGPGAPQGPGAPQGPGAPQGPGAPQGPGAPQGPGAPQGPGAPQGPGAPQGPGAPQGPGAPQGPGAPQGPGAPQGPGAPQGPGAPQGPGAPQEPPQQPPQQPPQQPPQQPPQQPPQQPNNNNNNNGNNNEDSYVPSAEQILEFVKQISSQLTEEWSQCSVTCGSGVRVRKRKNVNKQPENLTLEDIDTEICKMDKCSSIFNIVSNSLG

SEQ ID NO: 31 - protein, 943 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 32 - protein, 929 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 33 - protein, 935 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 34 - protein, 941 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 35 - protein, 947 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 36 - protein, 953 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 37 - protein, 959 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 38 - protein, 971 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPQGPGAPQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 39 - protein, 977 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEQGPGAPQGPGAPQGPGAPQGPGAPQGPGAPQGPGAPQGPGAPQGPGAPQGPGAPQGPGAPQGPGAPQGPGAPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRLLTPNEFEIKRSVDGEGYNDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 40 - protein, 975 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDEAATALEINLEEEDDDNEDEVDEQAEQQKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSASQGPGAPQGPGAPQGPGAPGTSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 41 - protein, 599 aa, Artificial Sequence
  Synthetic Construct
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGQGPGAPQGPGAPQGPGAPDTTPSAYSMSFSWDWSGHNYINEIFATSSYTFSYIAQE

SEQ ID NO: 42 - protein, 227 aa, Artificial Sequence
  Synthetic Construct
MSILISPSNNTGWGLRFPSKMFGGAKKRSDQHPVRVRGHYRAPWGAHKRGRTGRTTVDDAIDAVVEEARNYTPTPPPVSTVDAAIQTVVRGARRYAKMKRRRRRVARRHRRRPGTAAQRAAAALLNRARRTGRRAAMRAARRLAAGIVTVPPRSRRRAAAAAAAAISAMTQGRRGNVYWVRDSVSGLRVPVRTRPPRNVRTRPPRNYNRNIVNRLLGDALNGKPEEK

SEQ ID NO: 43 - protein, 374 aa, Artificial Sequence
  Synthetic Construct
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKC

SEQ ID NO: 44 - protein, 941 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQAVDSYDPDVRIIENHGTEDELPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 45 - protein, 929 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 46 - protein, 937 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 47 - protein, 945 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 48 - protein, 953 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 49 - protein, 961 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 50 - protein, 969 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 51 - protein, 977 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 52 - protein, 985 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 53 - protein, 993 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 54 - protein, 1017 aa, Artificial Sequence
  Synthetic Construct
MATPSMMPQWSYMHISGQDASEYLSPGLVQFARATETYFSLNNKFRNPTVAPTHDVTTDRSQRLTLRFIPVDREDTAYSYKARFTLAVGDNRVLDMASTYFDIRGVLDRGPTFKPYSGTAYNALAPKGAPNPCEWDENANPNANPNANPNANPNANPNANPNANPNANPNANPNANPNANPNANPNANPNANPNANPNANPNANPNANPNANPNANPNANPNANPNANPNANPNANPNANPNANPNANPKTHVFGQAPYSGINITKEGIQIGVEGQTPKYADKTFQPEPQIGESQWYETEINHAAGRVLKKTTPMKPCYGSYAKPTNENGGQGILVKQQNGKLESQVEMQFFSTTEAAAGNGDNLTPKVVLYSEDVDIETPDTHISYMPTIKEGNSRELMGQQSMPNRPNYIAFRDNFIGLMYYNSTGNMGVLAGQASQLNAVVDLQDRNTELSYQLLLDSIGDRTRYFSMWNQLPNYCFPLGGVINTETLTKVKPKTGQENGWEKDATEFSDKNEIRVGNNFAMEINLNANLWRNFLYSNIALYLPDKLKYSPSNVKISDNPNTYDYMNKRVVAPGLVDCYINLGARWSLDYMDNVNPFNHHRNAGLRYRSMLLGNGRYVPFHIQVPQKFFAIKNLLLLPGSYTYEWNFRKDVNMVLQSSLGNDLRVDGASIKFDSICLYATFFPMAHNTASTLEAMLRNDTNDQSFNDYLSAANMLYPIPANATNVPISIPSRNWAAFRGWAFTRLKTKETPSLGSGYDPYYTYSGSIPYLDGTFYLNHTFKKVAITFDSSVSWPGNDRLLTPNEFEIKRSVDGEGYNVAQCNMTKDWFLVQMLANYNIGYQGFYIPESYKDRMYSFFRNFQPMSRQVVDDTKYKDYQQVGILHQHNNSGFVGYLAPTMREGQAYPANFPYPLIGKTAVDSITQKKFLCDRTLWRIPFSSNFMSMGALTDLGQNLLYANSAHALDMTFEVDPMDEPTLLYVLFEVFDVVRVHQPHRGVIETVYLRTPFSAGNATT

SEQ ID NO: 55 - protein, 597 aa, Artificial Sequence
  Synthetic Construct
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTTPAPSPNCRLNAEKDAKLTLVLTKCGSQILATVSVLAVKGSLAPISGTVQSAHLIIRFDENGVLLNNSFLDPEYWNFRNGDLTEGTAYTNAVGFMPNLSAYPKSHGKTAKSNIVSQVYLNGDKTKPVTLTITLNGTQETGNANPNANPNANPNANPDTTPSAYSMSFSWDWSGHNYINEIFATSSYTFSYIAQE

SEQ ID NO: 56 - protein, 218 aa, Artificial Sequence
  Synthetic Construct
MSILISPSNNTGWGLRFPSKMFGGAKKRSDQHPVRVRGHYRAPWGAHKRGRTGRTTVDDAIDAVVEEARNYTPTPPPVSTVDAAIQTVVRGARRYAKMKRRRRRVARRHRRRPGTAAQRAAAALLNRARRTGRRAAMRAARRLAAGIVTVPPRSRRRAAAAAAAAISAMTQGRRGNVYWVRDSVSGLRVPVRTRPPRNEYLNKIQNSLSTEWSPCSVT

SEQ ID NO: 57 - protein, 246 aa, Artificial Sequence
  Synthetic Construct
MSILISPSNNTGWGLRFPSKMFGGAKKRSDQHPVRVRGHYRAPWGAHKRGRTGRTTVDDAIDAVVEEARNYTPTPPPVSTVDAAIQTVVRGAEYLNKIQNSLSTEWSPCSVTRRYAKMKRRRRRVARRHRRRPGTAAQRAAAALLNRARRTGRRAAMRAARRLAAGIVTVPPRSRRRAAAAAAAAISAMTQGRRGNVYWVRDSVSGLRVPVRTRPPRNVRTRPPRNYNRNIVNRLLGDALTRPPRN

SEQ ID NO: 58 - protein, 226 aa, Artificial Sequence
  Synthetic Construct
MSILISPSNNTGWGLRFPSKMFGGAKKRSDQHPVRVRGHYRAPWGAHKRGRTGRTTVDDAIDAVVEEARNYTPTPPPVSTVDAAIQTVVRGARRYAKMKRRRRRVARRHRRRPGTAAQRAAAALLNRARRTGRRAAMRAAEYLNKIQNSLSTEWSPCSVTRRLAAGIVTVPPRSRRRAAAAAAAAISAMTQGRRGNVYWVRDSVSGLRVPVRTRPPRNVRTRPPRN

SEQ ID NO: 59 - protein, 6 aa, Plasmodium yoelii
QGPGAP

SEQ ID NO: 60 - protein, 4 aa, Plasmodium falciparum
NANP

SEQ ID NO: 61 - protein, 21 aa, Plasmodium yoelii
YNRNIVNRLLGDALNGKPEEK

SEQ ID NO: 62 - protein, 20 aa, Plasmodium falciparum
EYLNKIQNSLSTEWSPCSVT

SEQ ID NO: 63 - protein, 9 aa, Plasmodium vivax
ANGAGNQPG

SEQ ID NO: 64 - protein, 4 aa, Plasmodium malariae
NAAG

SEQ ID NO: 65 - protein, 4 aa, Plasmodium yoelii
QQPP

SEQ ID NO: 66 - protein, 9 aa, Plasmodium yoelii
SYVPSAEQI